SEQUENCE LISTING (99 sequences)

SEQ ID NO: 1 - protein, 241 aa, Artificial Sequence
  NP_002497.2
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 2 - protein, 233 aa, Artificial Sequence
  NP_000585.2
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 3 - protein, 128 aa, Artificial Sequence
  MEDI-578 VH
QVQLVQSGAEVKKPGSSVKVSCKASGGTFSTYGISWVRQAPGQGLEWMGGIIPIFDTGNSAQSFQGRVTITADESTSTAYMELSSLRSEDTAVYYCARSSRIYDLNPSLTAYYDMDVWGQGTMVTVSS

SEQ ID NO: 4 - protein, 5 aa, Artificial Sequence
  MEDI-578 VHCDR1
TYGIS

SEQ ID NO: 5 - protein, 17 aa, Artificial Sequence
  MEDI-578 VHCDR2
GIIPIFDTGNSAQSFQG

SEQ ID NO: 6 - protein, 19 aa, Artificial Sequence
  MEDI-578 VHCDR3
SSRIYDLNPSLTAYYDMDV

SEQ ID NO: 7 - protein, 110 aa, Artificial Sequence
  MEDI-578 VL (1256A5 VL)
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAWVFGGGTKLTVL

SEQ ID NO: 8 - protein, 13 aa, Artificial Sequence
  MEDI-578 VLCDR1
SGSSSNIGNNYVS

SEQ ID NO: 9 - protein, 7 aa, Artificial Sequence
  MEDI-578 VLCDR2
DNNKRPS

SEQ ID NO: 10 - protein, 11 aa, Artificial Sequence
  MEDI-578 VLCDR3
GTWDSSLSAWV

SEQ ID NO: 11 - protein, 19 aa, Artificial Sequence
  SEQ ID NO 11
SSRIYDFNSALISYYDMDV

SEQ ID NO: 12 - protein, 19 aa, Artificial Sequence
  SEQ ID NO 12
SSRIYDMISSLQPYYDMDV

SEQ ID NO: 13 - protein, 467 aa, Artificial Sequence
  soluble TNFR2 amino acid sequence
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 731 aa, Artificial Sequence
  TNFR2-Fc_VH#4 - amino acid sequence
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVQSGAEVKKPGSSVKVSCKASGGTFSTYGISWVRQAPGQGLEWMGGIIPIFDTGNSAQSFQGRVTITADESTSTAYMELSSLRSEDTAVYYCARSSRIYDLNPSLTAYYDMDVWGQGTMVTVSSGGGGSGGGGSGGGGSAQSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAWVFGGGTKLTVL

SEQ ID NO: 15 - protein, 15 aa, Artificial Sequence
  (Gly4Ser)3 15 aa linker sequence
GGGGSGGGGSGGGGS

SEQ ID NO: 16 - protein, 2193 aa, Artificial Sequence
  TNFR2-Fc_VH#4 - nucleotide sequence
CTGCCCGCCCAGGTGGCCTTTACCCCTTATGCCCCCGAGCCCGGCAGCACCTGTCGGCTGAGAGAGTACTACGACCAGACCGCCCAGATGTGCTGCAGCAAGTGCTCTCCTGGCCAGCATGCCAAGGTGTTCTGCACCAAGACCAGCGACACCGTGTGCGACAGCTGCGAGGACAGCACCTACACCCAGCTGTGGAACTGGGTGCCCGAGTGCCTGAGCTGCGGCAGCAGATGCAGCAGCGACCAGGTGGAAACCCAGGCCTGCACCAGAGAGCAGAACCGGATCTGCACCTGTAGACCCGGCTGGTACTGCGCCCTGAGCAAGCAGGAAGGCTGCAGACTCTGCGCCCCTCTGCGGAAGTGCAGACCCGGCTTTGGCGTGGCCAGACCCGGCACCGAGACAAGCGACGTGGTCTGTAAGCCCTGCGCTCCTGGCACCTTCAGCAACACCACCAGCAGCACCGACATCTGCAGACCCCACCAGATCTGCAACGTGGTGGCCATCCCCGGCAACGCCAGCATGGATGCCGTCTGCACCAGCACTAGCCCCACCAGAAGTATGGCCCCTGGCGCCGTGCATCTGCCCCAGCCTGTGTCCACCAGAAGCCAGCACACCCAGCCCACCCCTGAGCCTAGCACCGCCCCCTCCACCAGCTTTCTGCTGCCTATGGGCCCTAGCCCTCCAGCCGAGGGAAGCACAGGCGACGAGCCCAAGAGCTGCGACAAGACCCACACCTGTCCCCCCTGCCCTGCCCCTGAACTGCTGGGCGGACCCAGCGTGTTCCTGTTCCCCCCAAAGCCCAAGGACACCCTGATGATCAGCCGGACCCCCGAAGTGACCTGCGTGGTGGTGGACGTGTCCCACGAGGACCCTGAAGTGAAGTTCAATTGGTACGTGGACGGCGTGGAAGTGCACAACGCCAAGACCAAGCCCAGAGAGGAACAGTACAACTCCACCTACCGGGTGGTGTCCGTGCTGACCGTGCTGCACCAGGACTGGCTGAACGGCAAAGAGTACAAGTGCAAGGTCTCCAACAAGGCCCTGCCTGCCCCCATCGAGAAAACCATCAGCAAGGCCAAGGGCCAGCCCCGCGAGCCTCAGGTGTACACACTGCCCCCCAGCCGGGAAGAGATGACCAAGAACCAGGTGTCCCTGACCTGCCTGGTCAAAGGCTTCTACCCCAGCGATATCGCCGTGGAATGGGAGAGCAATGGCCAGCCCGAGAACAACTACAAGACCACCCCCCCTGTGCTGGACAGCGACGGCTCATTCTTCCTGTACAGCAAGCTGACCGTGGACAAGAGCCGGTGGCAGCAGGGCAACGTGTTCAGCTGCAGCGTGATGCACGAGGCCCTGCACAACCACTACACCCAGAAGTCCCTGAGCCTGAGCCCCGGAAAGGGCGGTGGCGGATCCGGAGGTGGGGGATCTCAGGTGCAGCTGGTGCAGTCTGGCGCCGAAGTGAAGAAACCCGGCTCTAGCGTGAAGGTGTCCTGCAAGGCCAGCGGCGGCACCTTCTCCACCTACGGCATCAGCTGGGTCCGCCAGGCCCCTGGACAGGGCCTGGAATGGATGGGCGGCATCATCCCCATCTTCGACACCGGCAACAGCGCCCAGAGCTTCCAGGGCAGAGTGACCATCACCGCCGACGAGAGCACCTCCACCGCCTACATGGAACTGAGCAGCCTGCGGAGCGAGGACACCGCCGTGTACTACTGCGCCAGAAGCAGCCGGATCTACGACCTGAACCCCAGCCTGACCGCCTACTACGACATGGACGTGTGGGGCCAGGGCACCATGGTCACAGTGTCTAGCGGAGGCGGCGGATCTGGCGGCGGAGGAAGTGGCGGGGGAGGATCTGCCCAGAGCGTGCTGACCCAGCCCCCTTCTGTGTCTGCCGCCCCTGGCCAGAAAGTGACCATCTCCTGCAGCGGCAGCAGCAGCAACATCGGCAACAACTACGTGTCCTGGTATCAGCAGCTGCCCGGCACCGCCCCTAAGCTGCTGATCTACGACAACAACAAGCGGCCCAGCGGCATCCCCGACCGGTTTAGCGGCAGCAAGAGCGGGACTTCTGCTACACTGGGCATCACAGGCCTGCAGACCGGCGACGAGGCCGACTACTACTGCGGCACCTGGGACAGCAGCCTGAGCGCTTGGGTGTTCGGCGGAGGCACCAAGCTGACAGTGCTG

SEQ ID NO: 17 - protein, 735 aa, Artificial Sequence [sequence omitted]

SEQ ID NO: 18 - DNA, 2205 bp, Artificial Sequence
  TNFR2-Fc_varB - nucleotide sequence
ctgcccgcccaggtggcctttaccccttatgcccccgagcccggcagcacctgtcggctgagagagtactacgaccagaccgcccagatgtgctgcagcaagtgctctcctggccagcatgccaaggtgttctgcaccaagaccagcgacaccgtgtgcgacagctgcgaggacagcacctacacccagctgtggaactgggtgcccgagtgcctgagctgcggcagcagatgcagcagcgaccaggtggaaacccaggcctgcaccagagagcagaaccggatctgcacctgtagacccggctggtactgcgccctgagcaagcaggaaggctgcagactctgcgcccctctgcggaagtgcagacccggctttggcgtggccagacccggcaccgagacaagcgacgtggtctgcaagccctgcgctcctggcaccttcagcaacaccaccagcagcaccgacatctgcagaccccaccagatctgcaacgtggtggccatccccggcaacgccagcatggatgccgtgtgcaccagcaccagccccaccagaagtatggcccctggcgccgtgcatctgccccagcctgtgtccaccagaagccagcacacccagcccacccctgagcctagcaccgccccctccaccagctttctgctgcctatgggccctagccctccagccgagggaagcacaggcgacgagcccaagagctgcgacaagacccacacctgtcccccctgccctgcccctgaactgctgggcggacccagcgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtccgtgctgaccgtgctgcaccaggactggctgaacggcaaagagtacaagtgcaaagtctccaacaaggccctgcctgcccccatcgagaaaaccatcagcaaggccaagggccagccccgcgagcctcaggtgtacacactgccccccagccgggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggctcattcttcctgtacagcaagctgaccgtggacaagagccggtggcagcagggcaatgtcttcagctgtagcgtgatgcacgaggccctgcacaaccactacacccagaagtccctgagcctgagccccggaaagggcggaggcggatccggaggtgggggatctcaggtgcagctggtgcagtctggcgccgaagtgaagaaacccggctctagcgtgaaggtgtcctgcaaggccagcggcggcaccttctccacctacggcatcagctgggtccgccaggcccctggacagtgtctggaatggatgggcggcatcatccccatcttcgacaccggcaacagcgcccagagcttccagggcagagtgaccatcaccgccgacgagagcacctccaccgcctacatggaactgagcagcctgcggagcgaggacaccgccgtgtactactgcgccagaagcagccggatctacgacctgaaccccagcctgaccgcctactacgacatggacgtgtggggccagggcaccatggtcacagtgtctagcggaggcggaggcagcggaggtggtggatctggtggcggaggaagtggcggcggaggctctcagagcgtgctgacccagcccccttctgtgtctgccgcccctggccagaaagtgaccatctcctgcagcggcagcagcagcaacatcggcaacaactacgtgtcctggtatcagcagctgcccggcaccgcccctaagctgctgatctacgacaacaacaagcggcccagcggcatccccgaccggtttagcggcagcaagagcgggacttctgctacactgggcatcacaggcctgcagaccggcgacgaggccgactactactgcggcacctgggacagcagcctgagcgcttgggtgttcggctgcggcaccaagctgacagtgctg

SEQ ID NO: 19 - protein, 20 aa, Artificial Sequence
  (Gly4Ser)4 20 aa linker sequence
GGGGSGGGGSGGGGSGGGGS

SEQ ID NO: 20 - protein, 215 aa, Artificial Sequence
  ndimab varB - L chain amino acid sequence
EIVLTQSPATLSLSPGERATLSCRASQSVYSYLAWYQQKPGQAPRLLIYDASNRAIGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 645 bp, Artificial Sequence
  ndimab varB - L chain nucleotide sequence
gaaatcgtgctgacccagagccccgccaccctgtctctgagccctggcgagagagccaccctgagctgcagagccagccagagcgtgtactcctacctggcttggtatcagcagaagcccggccaggcccccagactgctgatctacgacgccagcaaccgggccatcggcatccctgccagattttctggcagcggcagcggcaccgacttcaccctgaccatcagcagcctggaacccgaggacttcgccgtgtactactgccagcagcggagcaactggccccccttcaccttcggccctggcaccaaggtggacatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 22 - protein, 724 aa, Artificial Sequence
  ndimab varB - H chain amino acid sequence
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGISAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLVQSGAEVKKPGSSVKVSCKASGGTFSTYGISWVRQAPGQCLEWMGGIIPIFDTGNSAQSFQGRVTITADESTSTAYMELSSLRSEDTAVYYCARSSRIYDLNPSLTAYYDMDVWGQGTMVTVSSGGGGSGGGGSGGGGSGGGGSQSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAWVFGCGTKLTVL

SEQ ID NO: 23 - DNA, 2172 bp, Artificial Sequence
  ndimab varB - H chain nucleotide sequence
caggtgcagctggtggaaagcggcggaggcgtggtgcagcccggcagaagcctgagactgagctgcgctgccagcggcttcatcttcagcagctacgccatgcactgggtccgccaggcccctggcaacggactggaatgggtggccttcatgagctacgacggcagcaacaagaagtacgccgacagcgtgaagggccggttcaccatcagccgggacaacagcaagaacaccctgtacctgcagatgaacagcctgcgggctgaggacaccgccgtgtactactgcgccagagaccgaggcatcagtgctggcggcaactactactactacggcatggacgtgtggggccagggcaccaccgtgaccgtgtctagcgcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaggcggagggggatccggcggagggggctctcaggtgcagctggtgcagtctggcgccgaagtgaagaaacccggctctagcgtgaaggtgtcctgcaaggccagcggcggcaccttctccacctacggcatcagctgggtccgccaggcccctggacagtgtctggaatggatgggcggcatcatccccatcttcgacaccggcaacagcgcccagagcttccagggcagagtgaccatcaccgccgacgagagcacctccaccgcctacatggaactgagcagcctgcggagcgaggacaccgccgtgtactactgcgccagaagcagccggatctacgacctgaaccccagcctgaccgcctactacgacatggacgtgtggggccagggcaccatggtcacagtgtctagcggaggcggaggcagcggaggtggtggatctggtggcggaggaagtggcggcggaggctctcagagcgtgctgacccagcccccttctgtgtctgccgcccctggccagaaagtgaccatctcctgcagcggcagcagcagcaacatcggcaacaactacgtgtcctggtatcagcagctgcccggcaccgcccctaagctgctgatctacgacaacaacaagcggcccagcggcatccccgaccggtttagcggcagcaagagcgggacttctgctacactgggcatcacaggcctgcagaccggcgacgaggccgactactactgcggcacctgggacagcagcctgagcgcttgggtgttcggctgcggcaccaagctgacagtgctg

SEQ ID NO: 24 - protein, 128 aa, Artificial Sequence
  NGF-NG VH amino acid sequence
QVQLVQSGAEVKKPGSSVKVSCKASGGTFWFGAFTWVRQAPGQGLEWMGGIIPIFGLTNLAQNFQGRVTITADESTSTVYMELSSLRSEDTAVYYCARSSRIYDLNPSLTAYYDMDVWGQGTMVTVSS

SEQ ID NO: 25 - DNA, 384 bp, Artificial Sequence
  NGF-NG VH nucleotide sequence
caggtgcagctggtgcagtctggggctgaggtgaagaagcctgggtcctcggtgaaggtctcctgcaaggcctctggaggcaccttctggttcggcgcgttcacctgggtgcgacaggcccctggacaaggacttgagtggatgggagggattattcctatcttcgggttgacgaacttggcacagaacttccagggcagagtcacgattaccgcggacgaatccacgagcacagtctacatggagctgagcagcttgagatctgaagacacggccgtatattattgtgcacgttcaagtcgtatctacgatctgaacccgtccctgaccgcctactacgatatggatgtctggggccaggggacaatggtcaccgtctcgagt

SEQ ID NO: 26 - protein, 110 aa, Artificial Sequence
  NGF-NG VL amino acid sequence
QSVLTQPPSVSAAPGQKVTISCSGSSSDIGNNYVSWYQQLPGTAPKLLIYDNNKRPSGIPDRFSGSKSGTSATLGITGLQTGDEADYYCGTWDSSLSAWVFGGGTKLTVL

SEQ ID NO: 27 - DNA, 330 bp, Artificial Sequence
  NGF-NG VL nucleotide sequence
cagtctgtgctgactcagccgccatcagtgtctgcggccccaggacagaaggtcaccatctcctgctctggaagcagctccgacattgggaataattatgtatcgtggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtgcttgggtgttcggcggagggaccaagctgaccgtccta

SEQ ID NO: 28 - protein, 126 aa, Artificial Sequence
  ndimab VH amino acid sequence
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGISAGGNYYYYGMDVWGQGTTVTVSS

SEQ ID NO: 29 - protein, 108 aa, Artificial Sequence
  ndimab VL amino acid sequence
EIVLTQSPATLSLSPGERATLSCRASQSVYSYLAWYQQKPGQAPRLLIYDASNRAIGIPARFSGSGSGTDFTLTISSLEPEDFAVYYCQQRSNWPPFTFGPGTKVDIK

SEQ ID NO: 30 - protein, 128 aa, Artificial Sequence
  1126F1 VH amino acid sequence
EVQLVQTGAEVKKPGSSVKVSCKASGGTFSTYGISWVRQAPGQGLEWIGGIIPIFDTGNSAQSFQGRVTITADESTSTAYMEVSSLRSDDTAVYYCASSSRIYDANRQAVPYYDMDVWGQGTMVTVSS

SEQ ID NO: 31 - protein, 110 aa, Artificial Sequence
  1126F1 VL amino acid sequence
QAVLTQPSSVSTPPGQMVTISCSGSSSDIGNNYVSWYQQLPGTAPKLLIYDNNKRPSGIPDRFSGSKSGTSATLGITGLQTGDEADYYCGTWDSSLSAWVFGGGTKLTVL

SEQ ID NO: 32 - protein, 128 aa, Artificial Sequence
  1126G5 VH amino acid sequence
EVQLVQSGAEVKKPGSSVKVSCKASGGTFSTYGISWVRQAPGQGLEWIGGIIPIFDTGNSAQSFQGRVTITADESTSTAYMEVSSLRSDDTAVYYCASSSRIYDFTSGLAPYYDMDVWGQGTMVTVSS

SEQ ID NO: 33 - protein, 110 aa, Artificial Sequence
  1126G5 VL amino acid sequence
QAVLTQPSSVSTPPGQKVTISCSGSSSNIGNNYVSWYQQLPGTAPKLLIYDNNKRPPGIPDRFSGSKSGTSATLGITGLQTGDEADYYCGTWDSSLSTWVFGGGTKLTVL

SEQ ID NO: 34 - protein, 128 aa, Artificial Sequence
  1126H5 VH amino acid sequence
EVQLVQSGAEVKKPGSSVKVSCKASGGTFSTYGISWVRQAPGQGLEWIGGIIPIFDAGNSAQSFQGRVTITADESTSTAHMEVSSLRSEDTAVYYCASSSRIYDHHIQKGGYYDMDVWGQGTMVTVSS

SEQ ID NO: 35 - protein, 110 aa, Artificial Sequence
  1126H5 VL amino acid sequence
QAVLTQPSSVSTPPGQKVTISCSGSSSNIGNNYVSWYQQLPGTAPKLLIYDNNKRPSGIPDRFSGSKSGTSATLGITGLQTGDEADYYCGTWDSSLSAWVFGGGTKLTVL

SEQ ID NO: 36 - protein, 112 aa, Artificial Sequence
  1127D9 VH amino acid sequence
EVQLVQSGAEVKKPGSSVKVSCKASGGTFSTYGISWVRQAPGQGLEWIGGIIPIFDTGNSAQSFQGRVTITADESTSTAYMEVSSLRSDDTAVYYCASSSRIYDYHTIAYYD

SEQ ID NO: 37 - protein, 110 aa, Artificial Sequence; identical to SEQ ID NO: 35
  1127D9 VL amino acid sequence

SEQ ID NO: 38 - protein, 128 aa, Artificial Sequence
  1127F9 VH amino acid sequence
EVQLVQSGAEVKKPGSSVKVSCKASGGTFSTYGISWVRQAPGQGLEWIGGIIPIFDTGNSAQSFQGRVTITADESTSTAYMKVSSLRSDDTAVYYCASSSRIYDYIPGMRPYYDMDVWGQGTMVTVSS

SEQ ID NO: 39 - protein, 110 aa, Artificial Sequence
  1127F9 VL amino acid sequence
QAVLTQPSSVSTPPGQKVTISCSGNSSNIGNNYVSWYQQLPGTAPKLLIYDNNKRPSGIPDRFSGSRSGTLATLGITGLQTGDEADYYCGTWDSSLSAWVFGGGTKLTVL

SEQ ID NO: 40 - protein, 128 aa, Artificial Sequence
  1131D7 VH amino acid sequence
EVQLVQSGAEVKKPGSSVKVSCKASGGTFSTYGISWVRQAPGQGLEWIGGIIPIFDTGNSAQSFQGRVTITADESTSTAYMEVSSLRSDDTAVYYCASSSRIYDFNSSLIAYYDMDVWGQGTMVTVSS

SEQ ID NO: 41 - protein, 110 aa, Artificial Sequence
  1131D7 VL amino acid sequence
QAVLTQPSSVSTPPGQKVTISCSGSSSNIGNNYVSWYQQLPGTAPKLLIYDNNKRPSGIPDRFSGSKSGTSATLGITGLQTGDETDYYCGTWDSSLSAWVFSGGTKLTVL

SEQ ID NO: 42 - protein, 128 aa, Artificial Sequence
  1131H2 VH amino acid sequence
EVQLVQSGAEVKKPGSTVKVSCKASGGTFSTYGISWVRQAPGQGLEWIGGIIPIFDTGNSAQSFQGRVTITADESTSTAYMEVSSLRSDDTAVYYCASSSRIYDLNPSLTAYYDMDVWGQGTMVTVSS

SEQ ID NO: 43 - protein, 110 aa, Artificial Sequence
  1131H2 VL amino acid sequence
QAVLTQPSSVSTPPGQKVTISCSGTSSNIGNNYVSWYQQLPGTAPKLLIYDNNKRPSGIPDRFSGSKSGTSATLGITGLQTGDEADYYCGTWDSSLSAWVFGGGTKLTVL

SEQ ID NO: 44 - protein, 128 aa, Artificial Sequence
  132A9 VH amino acid sequence
EVQLVQSGAEVKKPGSSVKVSCKASGGTFSTYGISWVRQAPGQGLEWIGGIIPIFGTGNSAQSFQGRVTITADESTSTAYMEVSSLRSDDTAVYYCASSSRIYDFEPSLIYYYDMDVWGQGTMVTVSS

SEQ ID NO: 45 - protein, 110 aa, Artificial Sequence; identical to SEQ ID NO: 35
  132A9 VL amino acid sequence

SEQ ID NO: 46 - protein, 128 aa, Artificial Sequence
  1132H9 VH amino acid sequence
EVQLVQSGAEVKKPGSSVKVSCKASGGTFSTYGISWVRQAPGQGLEWIGGIIPIFDTGNSAQSFQGRVTITADESTSTAYMEVSSLRSDDTAVYYCASSSRIYDLNPSLTAYYDMDVWGQGTMVTVSS

SEQ ID NO: 47 - protein, 110 aa, Artificial Sequence
  1132H9 VL amino acid sequence
QAVLTQPSSVSTPPGQKVTISCSGSSSDIGNNYVSWYQQLPGTAPKLLIYDNNKRPTGIPDRFSGSKSGTSATLGITGLQTGDEADYYCGTWDSSLSAWVFGGGTKLTVL

SEQ ID NO: 48 - protein, 128 aa, Artificial Sequence; identical to SEQ ID NO: 46
  1133C11 VH amino acid sequence

SEQ ID NO: 49 - protein, 110 aa, Artificial Sequence; identical to SEQ ID NO: 35
  1133C11 VL amino acid sequence

SEQ ID NO: 50 - protein, 128 aa, Artificial Sequence
  1134D9 VH amino acid sequence
EVQLVQSGAEVKKPGSSVKVSCKASGGTFSTYGISWVRQAPGQGLEWIGGIIPIFDTGNSAQSFQGRVAITADESTSTAYMEVSSLRSDDTAVYYCASSSRIYDLNPSLTAYYDMDVWGQGTMVTVSS

SEQ ID NO: 51 - protein, 110 aa, Artificial Sequence
  1134D9 VL amino acid sequence
QAVLTQPSSVSTPPGQKVTISCSGSSSNIGNNYVSWYQQLPGTAPKLLIYDNNKRPSGIPDRFSGSKSGTSATLGITGLQTGDEADYYCGTWDSGLSAWVFGGGTKLTVL

SEQ ID NO: 52 - protein, 128 aa, Artificial Sequence
  1145D1 VH amino acid sequence
EVQLVQSGAEVKKPGSSVKVSCKASGGTFSTYGISWVRQAPGQGLEWIGGIIPIFDTSNSAQSFQGRVTITADESTSTAYMEVSSLRSDDTAVYYCASSSRIYDFRTLYSTYYDMDVWGQGTMVTVSS

SEQ ID NO: 53 - protein, 110 aa, Artificial Sequence
  1145D1 VL amino acid sequence
QAVLTQPSSVSTPPGQKVTISCSGSSSNIGNNYVSWYQQLPGTAPKLLIYDNNKRPSGISDRFSGSKSGTSATLGIAGLQTGDEADYYCGTWDSSLSAWVFGGGTKLTVL

SEQ ID NO: 54 - protein, 128 aa, Artificial Sequence; identical to SEQ ID NO: 46
  1146D7 VH amino acid sequence

SEQ ID NO: 55 - protein, 110 aa, Artificial Sequence
  1146D7 VL amino acid sequence
QAVLTQPSSVSTPPGQEVTISCSGSSTNIGNNYVSWYQQLPGTAPKLLIYDNNKRPSGIPDRFSGSKSGTSATLGITGLQTGDEADYYCGTWDSSLSAWVFGGGTKLTVL

SEQ ID NO: 56 - protein, 128 aa, Artificial Sequence
  1147D2 VH amino acid sequence
EVQLVQSGAEVKKPGSSVRISCKASGGTFSTYGVSWVRQAPGQGLEWIGGIIPIFDTGNSAQSFQGRVTITADESTSTAYMEVSSLRSDDTAVYYCASSSRIYDLNPSLTAYYDMDVWGQGTMVTVSS

SEQ ID NO: 57 - protein, 110 aa, Artificial Sequence
  1147D2 VL amino acid sequence
QAVLTQPSSVSTPPGQKVTISCSGSSSNIGNNYVSWYQQLPGTAPKLLIYDNNKRPSGVPDRFSGSKSGTSATLGITGLQTGDEADYYCGTWDSSLSAWVFGGGTKLTVL

SEQ ID NO: 58 - protein, 126 aa, Artificial Sequence
  1147G9 VH amino acid sequence
EVQLVQSGAEVKKPGSSVKVSCKASGGTFSAYGISWVRQAPGQGLEWIGGIIPIFNTGNSAQSFQGRVTITADESTSTAYMEVSSLRSDDTAVYYCASSSRIYDLNPSLTAYYDMDVWGQGTMVTV

SEQ ID NO: 59 - protein, 110 aa, Artificial Sequence
  1147G9 VL amino acid sequence
QAVLTQPSSVSTPPGQKVTVSCSGSSSNIGNNYVSWYQQLPGTAPKLLIYDNNKRPSGIPDRFSGSKSGTSATLGITGLQTGDEADYYCGTWDSSLSAWVFGGGTKLTVL

SEQ ID NO: 60 - protein, 128 aa, Artificial Sequence
  1150F1 VH amino acid sequence
EVQLVQSGAEVKKPGSSVKVSCKASGGTFSTYGISWVRQAPGQGLEWIGGIIPIFDTGNSAQSFQDRVTITADESTSTAYMEVGSLRSDDTAVYYCASSSRIYDLNPSLTAYYDMDVWGHGTMVTVSS

SEQ ID NO: 61 - protein, 110 aa, Artificial Sequence; identical to SEQ ID NO: 35
  1150F1 VL amino acid sequence

SEQ ID NO: 62 - protein, 128 aa, Artificial Sequence
  1152H5 VH amino acid sequence
EVQLVQSGAEVKKPGSSVKVSCKASGGTFSTYGISWVRQAPGQGLVWIGGIIPIFDTGNSAQSFQGRVTITADESTSTAYMEVSSLRSDDTAVYYCASSSRIYDMISSLQPYYDMDVWGQGTMVTVSS

SEQ ID NO: 63 - protein, 110 aa, Artificial Sequence
  1152H5 VL amino acid sequence
QAVLTQPSSVSTPPGQKATISCSGSSSNIGNNYVSWYQQLPGTAPKLLIYDNNKRPSGIPDRFSGSKSGTSATLGITGLQTGDEADYYCGTWDSSLSAWVFGGGTKLTVL

SEQ ID NO: 64 - protein, 128 aa, Artificial Sequence
  1155H1 VH amino acid sequence
EVQLVQSGAEVKKPGSSVKVSCKASGGTFSTYGISWVRQAPGQGLEWIGGIIPIFDTGNSAQSFQGRVTITADESTSTAYMEVSSLRSDDTAVYYCASSSRIYDFHLANKGYYDMDVWGQGTMVTVSS

SEQ ID NO: 65 - protein, 110 aa, Artificial Sequence
  1155H1 VL amino acid sequence
QAVLTQPSSVSTPPGQKATISCSGSSSNIGNNYVSWYQQLPGTAPKLLIYDNNKRPSGIPDRFSGSKSGTSATLDITGLQTGDEADYYCGTWDSSLSAWVFGGGTKLTVL

SEQ ID NO: 66 - protein, 128 aa, Artificial Sequence
  1158A1 VH amino acid sequence
EVQLVQSGAEVKKPGSSVKVSCKASGGTFSTYGISWVRQAPGQGLEWIGGIIPIFGTGNSAQSFQGRVTITADESTSTAYMEVSSLRSDDTAVYYCASSSRIYDHHNHVGGYYDMDVWGQGTMVTVSS

SEQ ID NO: 67 - protein, 110 aa, Artificial Sequence
  1158A1 VL amino acid sequence
QAVLTQPSSVSTPPGQKVTISCSGSSSNIGNNYASWYQQLPGTAPKLLIYDNNKRPSGIPDRFSGSKSGTSATLGITGLQTGDEADYYCGTWDGSLSAWVFGGGTKLTVL

SEQ ID NO: 68 - protein, 128 aa, Artificial Sequence
  1160E3 VH amino acid sequence
EVQLVQSGAEVKKPGSSAKVSCKASGGTFSTYGISWVRQAPGQGLEWIGGIIPIFDTGNSAQSFQGRVTITADESTSTAYMEVSSLRSDDTAVYYCASSSRIYDLNPSLTAYYDMDVWGQGTMVTVSS

SEQ ID NO: 69 - protein, 109 aa, Artificial Sequence
  1160E3 VL amino acid sequence
QAVLTQPSSVSTPPGQKVTISCSGSNSNIGNNYVSWYQQLPGTAPKLLIYDNNKRPSGIPDRFSGSKSGTSATLGITGLQTGDEADYYCGTWDSSLSAWVFGGGTKLTV

SEQ ID NO: 70 - protein, 128 aa, Artificial Sequence; identical to SEQ ID NO: 46
  1165D4 VH amino acid sequence

SEQ ID NO: 71 - protein, 110 aa, Artificial Sequence
  1165D4 VL amino acid sequence
QAVLTQPSSVSTPPGQKVTISCSGSSSNIENNYVSWYQQLPGTAPKLLIYDNNKRPSGIPDRFSGSKSGTSATLGITGLQTGDEADYYCGTWDSSLSAWVFGGGTKLTVL

SEQ ID NO: 72 - protein, 128 aa, Artificial Sequence
  1175H8 VH amino acid sequence
EVQLVQSGAEVKKPGSSVKVSCKASGGTFSTYGISWVRQAPGQRLEWIGGIIPIFDTGNSAQSFQGRVTITADESTSTAYMEVSSLRSDDTAVYYCASSSRIYDATTGLTPYYDMDVWGQGTMVTVSS

SEQ ID NO: 73 - protein, 110 aa, Artificial Sequence
  1175H8 VL amino acid sequence
QAVLTQPSSVSTPPGQKVTISCSGSSSNIGNNYVSWYQQLPGTAPKLLIYDNNKRPSGIPDRFSGSKSGTSATLGITGLRTGDEADYYCGTWDSSLSAWVFGGGTKLTVL

SEQ ID NO: 74 - protein, 128 aa, Artificial Sequence
  1211G10 VH amino acid sequence
EVQLVQSGAEVRKPGSSVKVSCKAYGGTFSTYGISWVRQAPGQGLEWVGGIIPIFDTRNSAQSFQGRVTITADESTSTAYMEVSSLRSDDTAVYYCASSSRIYDMVSTLIPYYDMDVWGQGTMVTVSS

SEQ ID NO: 75 - protein, 110 aa, Artificial Sequence; identical to SEQ ID NO: 35
  1211G10 VL amino acid sequence

SEQ ID NO: 76 - protein, 126 aa, Artificial Sequence
  1214A1 VH amino acid sequence
EVQLVQSGAEVKKPGSSVRVSCKASGGTFSTYGISWVRQAPGQGLEWIGGIIPIFDTGNSAQSFQGRVTITADESTSTAYMEVSSLRSDDTAVYYCASSSRIYDAHLQAYYDMDVWGQGTMVTVSS

SEQ ID NO: 77 - protein, 110 aa, Artificial Sequence
  1214A1 VL amino acid sequence
QAVLTQPSSVSTPPGQKVTISCSGSSSNIGNNYVSWYQQLPGTAPKLLIYDNNKRPPGIPDRFSGSKSGTSATLGITGLQTGDEADYYCGTRDSSLSAWVFGGGTKLTVL

SEQ ID NO: 78 - protein, 128 aa, Artificial Sequence
  1214D10 VH amino acid sequence
EVQLVQSGAEAKKPGSSVKVSCKASGGTFSTYGISWVRQAPGRGLEWIGGIIPIFDTGNSAQSFQGRVAITADESTSTAYMEVSSLRSDDTAVYYCASSSRIYDAHLNHHGYYDMDVWGQGTMVTVSS

SEQ ID NO: 79 - protein, 110 aa, Artificial Sequence
  1214D10 VL amino acid sequence
QAVLTQPSSVSTPPGQKVTISCSGSSSNIGNNYVSWYQQLPGTAPKLLIYDNNKRPSGIPDRFSGSKSGTSATLGITGLQAGDEADYYCGTWDSSLSAWVFGGGTKLTVL

SEQ ID NO: 80 - protein, 128 aa, Artificial Sequence
  1218H5 VH amino acid sequence
EVQLVQSGAVVKKPGSSVKVSCKASGGTFSTYGISWVRQAPGQGLEWIGGIIPIFDTGSSAQSFQGRVTITADESTSTAYMEVSSLRSDDTAVYYCASSSRIYDLNPSLTAYYDMDVWGQGTMVTVSS

SEQ ID NO: 81 - protein, 110 aa, Artificial Sequence
  1218H5 VL amino acid sequence
QAVLTQPSSVSTPPGQKVTISCSGSSSNTGNNYVSWYQQLSGTAPKLLIYDNNKRPSGIPDRFSGSKSGTSATLGITGLQTGDEADYYCGTWDSSLSAWVFGGGTKLTVL

SEQ ID NO: 82 - protein, 128 aa, Artificial Sequence
  1230H7 VH amino acid sequence
EMQLVQSGAEVKKPGSSVKVSCKASGGTFSTYGISWVRQAPGQGLEWIGGIIPIFDTGNSAQSFQGRVTITADESTSTAYMEVSSLRSDDTAVYYCASSSRIYDFNSALISYYDMDVWGQGTMVTVSS

SEQ ID NO: 83 - protein, 109 aa, Artificial Sequence
  1230H7 VL amino acid sequence
QAVLTQPSSVSTPPGQKVTISCSGSSSNIGNNYVSWYQQLPGTAPKLLIYDNNKRPSGIPDRFSGSKSGTSATLGITGLQTGDEADYYCGTWDSSLSAWVFGGGTKLTV

SEQ ID NO: 84 - protein, 125 aa, Artificial Sequence
  1083H4 VH amino acid sequence
QMQLVQSGAEVKKTGSSVKVSCKASGYTFAYHYLHWVRQAPGQGLEWMGGIIPIFGTTNYAQRFQDRVTITADESTSTAYMELSSLRSEDTAVYYCASADYVWGSYRPDWYFDLWGRGTMVTVSS

SEQ ID NO: 85 - protein, 110 aa, Artificial Sequence
  1083H4 VL amino acid sequence
QSVLTQPPSASGTPGQRVTISCSGSSSNIGSNTVNWYQRLPGAAPQLLIYNNDQRPSGIPDRFSGSKSGTSGSLVISGLQSEDEADYYCASWDDSLNGRVFGGGTKLTVL

SEQ ID NO: 86 - protein, 125 aa, Artificial Sequence
  1227H8 VH amino acid sequence
QMQLVQSGAEVKKTGSSVKVSCKASGHTFAYHYLHWVRQAPGQGLEWMGGIIPIFGTTNYAQRFQDRVTITADESTSTAYMELSSLRSEDTAVYYCASADYAWESYQPPQINGVWGRGTMVTVSS

SEQ ID NO: 87 - protein, 110 aa, Artificial Sequence
  1227H8 VL amino acid sequence
QSVLTQPPSVSAAPGQKVTITCSGSTSNIGNNYVSWYQQHPGKAPKLMIYDVSKRPSGVPDRFSGSKSGNSASLDISGLQSEDEADYYCAAWDDSLSEFFFGTGTKLTVL

SEQ ID NO: 88 - protein, 5 aa, Artificial Sequence
  NGF-NG HCDR1
FGAFT

SEQ ID NO: 89 - protein, 17 aa, Artificial Sequence
  NGF-NG HCDR2
GIIPIFGLTNLAQNFQG

SEQ ID NO: 90 - protein, 19 aa, Artificial Sequence
  NGF-NG HCDR3
SSRIYDLNPSLTAYYDMDV

SEQ ID NO: 91 - protein, 13 aa, Artificial Sequence
  NGF-NG LCDR1
SGSSSDIGNNYVS

SEQ ID NO: 92 - protein, 7 aa, Artificial Sequence
  NGF-NG LCDR2
DNNKRPS

SEQ ID NO: 93 - protein, 11 aa, Artificial Sequence
  NGF-NG LCDR3
GTWDSSLSAWV

SEQ ID NO: 94 - protein, 128 aa, Artificial Sequence
  MEDI-578 VH amino acid sequence with G->C
QVQLVQSGAEVKKPGSSVKVSCKASGGTFSTYGISWVRQAPGQCLEWMGGIIPIFDTGNSAQSFQGRVTITADESTSTAYMELSSLRSEDTAVYYCARSSRIYDLNPSLTAYYDMDVWGQGTMVTVSS

SEQ ID NO: 95 - protein, 110 aa, Artificial Sequence
  MEDI-578 VL amino acid sequence with G->C
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAWVFGCGTKLTVL

SEQ ID NO: 96 - protein, 125 aa, Artificial Sequence
  1230D8 VH amino acid sequence
QMQLVQSGAEVKKTGSSVKVSCKASGYTFPYHYLHWVRQAPGQGLEWMGGIIPIFGTTNYAQRFQDRVTITADESTSTAYMEFSSLRSEDTAVYYCASADYVWESYHPATSLSLWGRGTMVTVSS

SEQ ID NO: 97 - protein, 110 aa, Artificial Sequence
  1230D8 VL amino acid sequence
QSVLTQPPSVSAAPGQKVTISCPGSTSNIGNNYVSWYQQRPGKAPKLMIYDVSKRPSGVPDRFSGSKSGNSASLDISELQSEDEADYYCAAWDDSLSEFLFGTGTKLTVL

SEQ ID NO: 98 - protein, 10 aa, Artificial Sequence
  Linker
GGGGSGGGGS

SEQ ID NO: 99 - DNA, 2205 bp, Artificial Sequence
  TNFR2-Fc_varB codon optimized nucleotide sequence
ctgcccgcccaggtggcctttaccccttatgctcctgagcccggctctacctgccggctgagagagtactacgaccagaccgcccagatgtgctgctccaagtgctctcctggccagcacgccaaggtgttctgcaccaagacctccgataccgtgtgcgactcctgcgaggactccacctacacccagctgtggaactgggtgcccgagtgcctgtcctgcggctccagatgttcctccgaccaggtggaaacccaggcctgcaccagagagcagaaccggatctgcacctgtcggcctggctggtactgcgccctgtctaagcaggaaggctgcagactgtgcgcccctctgcggaagtgtagacctggctttggcgtggccagacccggcaccgagacatctgatgtcgtgtgcaagccttgcgcccctggcaccttctccaacaccacctcctccaccgacatctgccggcctcaccagatctgcaacgtggtggccatccctggcaacgcctctatggacgccgtgtgcacctctacctcccccaccagaagtatggcccctggcgctgtgcatctgccccagcctgtgtctaccagatcccagcacacccagcccacccctgagccttctaccgccccttctaccagcttcctgctgcctatgggccctagccctcctgctgagggatctacaggcgacgagcccaagtcctgcgacaagacccacacctgtcccccttgtcctgcccctgaactgctgggcggaccttccgtgttcctgttccccccaaagcccaaggacaccctgatgatcagccggacccctgaagtgacctgcgtggtggtggatgtgtcccacgaggatcccgaagtgaagttcaattggtacgtggacggcgtggaagtgcacaacgccaagaccaagcccagagaggaacagtacaactccacctaccgggtggtgtccgtgctgaccgtgctgcaccaggattggctgaacggcaaagagtacaagtgcaaggtgtccaacaaggccctgcctgcccccatcgaaaagaccatctccaaggccaagggccagccccgggaaccccaggtgtacacactgccccctagccgggaagagatgaccaagaaccaggtgtccctgacctgtctcgtgaagggcttctacccctccgatatcgccgtggaatgggagtccaacggccagcctgagaacaactacaagaccaccccccctgtgctggactccgacggctcattcttcctgtactccaagctgacagtggacaagtcccggtggcagcagggcaacgtgttctcctgctccgtgatgcacgaggccctgcacaaccactacacccagaagtccctgtccctgagccctggaaaaggcggcggaggatctggcggaggcggatctcaggtgcagctggtgcagtctggcgctgaagtgaagaaacccggctcctccgtgaaggtgtcctgcaaggcttctggcggcaccttctctacctacggcatctcctgggtgcgacaggcccctggccagtgcctggaatggatgggcggcatcatccccatcttcgacaccggcaactccgcccagagcttccagggcagagtgaccatcaccgccgacgagtctacctccaccgcctacatggaactgtcctccctgcggagcgaggacaccgccgtgtactactgcgcccggtcctctcggatctacgacctgaacccttccctgaccgcctactacgacatggacgtgtggggccagggcacaatggtcaccgtgtcatctggtggtggcggctctggtggcggaggaagtgggggagggggttctggggggggaggatctcagtctgtgctgacccagcctccttccgtgtctgctgccccaggccagaaagtgacaatctcctgcagcggctccagctccaacatcggcaacaactacgtgtcctggtatcagcagctgcccggcaccgctcccaaactgctgatctacgataacaacaagcggccctccggcatccccgacagattctccggctctaagtccggcacctctgccaccctgggcatcaccggactgcagacaggcgacgaggccgactactactgtggcacctgggactcctccctgtccgcttgggtgttcggctgcggcaccaaactgactgtgctg

(19) United States
(12) Patent Application Publication
(10) Pub. No.: US 2024/0026036 A1
(43) Pub. Date: Jan. 25, 2024

(54) COMPOUNDS AND METHODS FOR TREATING PAIN

(71) Applicant: MEDIMMUNE LIMITED, CAMBRIDGE (GB)

(72) Inventors: FRASER WELSH, CAMBRIDGE (GB); THARANI CHESSELL, CAMBRIDGE (GB); KEITH TAN, CAMBRIDGE (GB); THOR OSTENFELD, CAMBRIDGE (GB); RICHARD JENKINS, CAMBRIDGE (GB)

(21) Appl. No.: 18/246,794

(22) PCT Filed: Sep. 27, 2021
(86) PCT No.: PCT/EP2021/076524

§ 371 Date: Mar. 27, 2023

Related U.S. Application Data
(60) Provisional application No. 63/084,358, filed on Sep. 28, 2020.

(51) Int. Cl. C07K 16/46; C07K 16/24; C07K 16/22; A61P 29/00

(52) U.S. Cl. CPC C07K 16/468; C07K 16/241; C07K 16/22; A61P 29/00; A61K 2039/545

(57) ABSTRACT

The disclosure provides novel methods and dosage regimens for use in treating or preventing pain, wherein the binding molecule comprises an NGF antagonist domain and a TNFα antagonist domain, wherein the NGF antagonist domain is an anti-NGF antibody or an antigen-binding fragment thereof and wherein the TNFα antagonist domain comprises a soluble TNFα binding fragment of TNFR.

### RELATED APPLICATIONS

The present application is a 35 U.S.C. § 371 national stage filing of International Application No. PCT/RP2021/076524, filed Sep. 27, 2021, which in turn claims benefit of U.S. Provisional Application Ser. No. 63/084,358, filed Sep. 28, 2020. The contents of each of the foregoing applications are incorporated herein by reference in their entirety.

### SEQUENCE LISTING

The instant applications contains a Sequence Listing which has been submitted electronically in ASCII format and is hereby incorporated by reference in its entirety. Said ASCII copy, created on Oct. 10, 2023, is named 132145-00401_SL and is 136,744 bytes in size.

### BACKGROUND

Pain is one of the most common symptoms for which medical assistance is sought and is the primary complaint of half of all patients visiting a physician. Despite the existence and widespread use of numerous pain medications, the elimination of pain, particularly chronic pain, has been without success. Thus, the burden on society remains high. Various studies estimate that pain results in 50 million workdays lost each year and S61.2 billion in lost productivity. For chronic pain sufferers, only about half are able to manage pain with the available prescribed treatment options. And, the total prescription pain medication market is approximately S25 billion per year.

Pain is the dominant symptom of osteoarthritis, which is a leading cause of disability and source of societal cost in older adults. With an ageing and increasingly obese population, this syndrome is becoming even more prevalent than in previous decades (Hunter & Bierma-Zeinstra Lancet, 393:1745-59 (2019)). Current treatments for pain in osteoarthritis include low-doses of oral NSAIDs. However, due to their association with increased mortality rates due to cardiovascular events, NSAID use is preferably restricted to short-term use (Kolasinski et al., Arthritis Care & Research, 72(2) 149-162 (2020)). As is suggested by these data, a large need remains for safe and effective novel analgesics.

Therapeutic agents that reduce the tissue levels or inhibit the effects of secreted nerve growth factor (NGF or beta-NGF) have the potential to be just such novel analgesics. NGF plays a well-known pivotal role in the development of the nervous system; however, NGF is also a well-validated target for pain as it causes pain in animals and humans. In adults, NGF, in particular, promotes the health and survival of a subset of central and peripheral neurons (Huang & Reichardt, Ann. Rev. Neurosci. 24:677-736 (2001)). NGF also contributes to the modulation of the functional characteristics of these neurons and exerts tonic control over the sensitivity, or excitability, of sensory pain receptors called nociceptors (Priestley et al., Can. J. Physiol. Pharmacol. 80:495-505 (2002); Bennett, Neuroscientist 7:13-17 (2001)). Nociceptors sense and transmit to the central nervous system the various noxious stimuli that give rise to perceptions of pain (nociception). NGF receptors are located on nociceptors. The expression of NGF is increased in injured and inflamed tissue and is upregulated in human pain states. Thus, because of NGF's role in nociception, NGF-binding agents that reduce levels of NGF possess utility as analgesic therapeutics.

Tumor necrosis factor-alpha (TNFα), also called cachectin, is a pleiotropic cytokine with a broad range of biological activities including cytotoxicity, immune cell proliferation, inflammation, tumorigenesis, and viral replication. Kim et al., J. Mol. Biol. 374, 1374 (2007). TNFα is first produced as a transmembrane protein (tm TNFα), which is then cleaved by a metalloproteinase to a soluble form (sTNFα). Wallis, Lancet Infect. Dis. 8(10): 601 (2008). TNFα (˜17 kDa) exists as a rigid homotrimeric molecule, which binds to cell-surface TNF Receptor 1 or TNF Receptor 2, inducing receptor oligomerization and signal transduction. Inflammatory cytokines, and in particular TNFα, are known to have a role in the generation of hyperalgesia. Leung, L., and Cahill, CM., J. Neuroinflammation 7:27 (2010). Some preliminary data has shown that TNFα inhibitors may be useful in the control of neuropathic pain. See, e.g., Sommer C, et al., J. Peripher. Nerv. Syst. 6:67-72 (2001), Cohen et al, A&A February 2013, 116, 2, 455-462, Genevay et al., Ann Rheum Dis 2004, 63, 1120-1123. The results from clinical studies testing TNFα inhibitors as a single therapy in the treatment of neuropathic pain remain inconclusive. See Leung and Cahill (2010).

A previously disclosed binding molecule comprising an anti-NGF antigen binding fragment and a soluble TNFR-2 portion was shown to be a potent inhibitor of both NGF and TNFα. Moreover, this binding molecule was shown therapeutically efficacious in reducing signs of pain in an animal model of pain. See, e.g., U.S. Pat. No. 9,884,911, which is incorporated by reference in its entirety. In view of the clear therapeutic utility of these binding molecules, there is a need for improved dosage regimens for binding molecules for treating, such as reducing or preventing, pain (e.g., osteoarthritic pain) in a subject in need thereof.

### SUMMARY OF THE INVENTION

This disclosure provides novel methods and dosage regimens for treating pain, such as for reducing or preventing pain in a subject, comprising administering to the subject a subcutaneous fixed dose of a binding molecule, wherein the binding molecule comprises an NGF antagonist portion and a TNFα antagonist portion. In some embodiments, the administration controls pain in the subject more effectively than an equivalent amount of the NGF antagonist or the TNFα antagonist administered alone.

In some embodiments, the disclosure provides for a method for reducing or preventing pain in a subject in need thereof, comprising administering to the subject a subcutaneous fixed dose of a binding molecule, wherein the binding molecule comprises an NGF antagonist domain and a TNFα antagonist domain, wherein the NGF antagonist domain is an anti-NGF antibody or an antigen-binding fragment thereof, wherein the TNFα antagonist domain comprises a soluble TNFα binding fragment of TNFR, and wherein the method reduces or prevents pain in the subject. In some embodiments, the subcutaneous fixed dose of the binding molecule is 5-200 mg. In some embodiments, the subcutaneous fixed dose of the binding molecule is 7.5-150 mg. In some embodiments, the subcutaneous fixed dose of the binding molecule is 7.5, 25, 75, or 150 mg. In some embodiments, the subcutaneous fixed dose is equivalent to an intravenous fixed dose of 30 mg of the binding molecule. In some embodiments, the fixed dose is administered at least every two weeks. In some embodiments, the fixed dose is administered for at least 12 weeks. In some embodiments, the pain comprises chronic pain. In some embodiments, the pain comprises osteoarthritic pain. In some embodiments, the pain comprises osteoarthritic pain of the knee.

In some embodiments, the subject has suffered the pain for 3 months or longer prior to administration with the binding molecule. In some embodiments, the pain is associated with joint inflammation. In some embodiments, the subject has osteoarthritis. In some embodiments, the subject has unilateral osteoarthritis of the knee. In some embodiments, the subject has Grade 2 osteoarthritis of the knee joint on the Kellgren-Lawrence (KL) grading scale of 0 to 4 as per central reader evaluation.

In some embodiments, the method comprises, prior to administration of the binding molecule to the subject: a. administering to the subject a NSAID, strong opiod, weak opioid, COX-2 inhibitor, acetaminophen or a combination thereof, and b. determining i) that the NSAID, strong opioid, weak opioid, COX-2 inhibitor, acetaminophen or a combination thereof does not reduce or prevent pain in the subject, and/or ii) determining that the subject is intolerant to the NSAID, strong opioid, weak opioid, COX-2 inhibitor, acetaminophen or a combination thereof. In some embodiments, the NSAID, strong opioid, weak opioid, COX-2 inhibitor, acetaminophen or a combination thereof is administered for at least 2 weeks. In some embodiments, the NSAID, strong opioid, weak opioid, COX-2 inhibitor, acetaminophen or a combination thereof has been administered to the subject for at least 2 weeks prior to administration with the binding molecule. In some embodiments, the subject is intolerant to NSAIDs, strong opioids, weak opioids, COX-2 inhibitors, acetaminophen (paracetamol) or a combination thereof.

In some embodiments, the method comprises testing the subject for SARS-CoV2 infection prior to administration with the fixed dose of the binding molecule. In some embodiments, testing the subject for SARS-CoV2 infection comprises testing the subject for SAR-CoV2 genetic material prior to administration with the fixed dose of the binding molecule. In some embodiments, the subject is not infected with SARS-CoV2 at baseline.

In some embodiments, the subject has a mean pain intensity score of at least 5 in a joint as measured on a pain numerical rating scale (NRS) at baseline. In some embodiments, the method reduces the subject's weekly average of daily NRS pain score from baseline. In some embodiments, the fixed dose is administered every 2 weeks for 12 weeks, and wherein the method reduces the subject's weekly average of daily NRS pain score from baseline by at least week 12. In some embodiments, the method reduces the subject's weekly average of daily NRS pain score from baseline by at least 30%. In some embodiments, the method reduces the subject's weekly average of daily NRS pain score from baseline by at least 50%.

In some embodiments, the subject has a mean Western Ontario and McMaster Universities Osteoarthritis (WOMAC) pain score of at least 5 in a joint as measured using the pain subscale of the WOMAC index at baseline. In some embodiments, the method reduces the subject's WOMAC pain subscale score from baseline. In some embodiments, the fixed dose is administered every 2 weeks for 12 weeks, and the method reduces the subject's weekly average of daily WOMAC pain score from baseline by at least week 12. In some embodiments, the method reduces the subject's WOMAC pain subscale score from baseline by at least 30%. In some embodiments, the method reduces the subject's WOMAC pain subscale score from baseline by at least 50%. In some embodiments, the method reduces the subject's WOMAC physical subscale score from baseline by at least 30%. In some embodiments, the method reduces the subject's WOMAC physical subscale score from baseline by at least 50%.

In some embodiments, the method improves the Patient Global Assessment (PGA) of osteoarthritis from baseline. In some embodiments, the fixed dose is administered every 2 weeks for 12 weeks, and wherein method reduces the PGA of osteoarthritis from baseline by at least week 12. In some embodiments, the method improves the PGA of osteoarthritis by at least 2 points.

In some embodiments, pain reduction is observed following a single dose administration of the binding molecule in the subject. In some embodiments, the method comprises administering an NSAID to the subject. In some embodiments, the method comprises administering an opioid to the subject. In some embodiments, the method comprises administering paracetamol to the subject. In some embodiments, the method comprises administering a COX-2 inhibitor to the subject.

In some embodiments, the anti-NGF antibody or fragment thereof can inhibit NGF binding to TrkA, p75NRT, or both TrkA and P75NRT. In some embodiments, the anti-NGF antibody or fragment thereof preferentially blocks NGF binding to TrkA over NGF binding to p75NRT. In some embodiments, the anti-NGF antibody or fragment thereof binds human NGF with an affinity of about 0.25-0.44 nM. In some embodiments, the anti-NGF antibody or fragment thereof comprises an antibody VH domain comprising a set of CDRs HCDR1, HCDR2, HCDR3 and an antibody VL domain comprising a set of CDRs LCDR1, LCDR2 and LCDR3, wherein the HCDR1 has the amino acid sequence of SEQ ID NO: 4 or SEQ ID NO: 4 with up to two amino acid substitutions, the HCDR2 has the amino acid sequence of SEQ ID NO: 5 or SEQ ID NO: with up to two amino acid substitutions, the HCDR3 has the amino acid sequence of SEQ ID NO: 6 or SEQ ID NO: 6 with up to two amino acid substitutions, SSRIYDFNSALISYYDMDV (SEQ ID NO: 11), or SSRIYDMISSLQPYYDMDV (SEQ ID NO:12), the LCDR1 has the amino acid sequence of SEQ ID NO: 8 or SEQ ID NO: 8 with up to two amino acid substitutions, the LCDR2 has the amino acid sequence of SEQ ID NO: 9 or SEQ ID NO: 9 with up to two amino acid substitutions, and the LCDR3 has the amino acid sequence of SEQ ID NO: 10 or SEQ ID NO: 10 with up to two amino acid substitutions. In some embodiments, the anti-NGF antibody or fragment thereof comprises an antibody VH domain comprising a set of CDRs HCDR1, HCDR2, HCDR3 and an antibody VL domain comprising a set of CDRs LCDR1, LCDR2 and LCDR3, wherein the HCDR1 comprises the amino acid sequence of SEQ ID NO: 4, the HCDR2 comprises the amino acid sequence of SEQ ID NO: 5, the HCDR3 comprises the amino acid sequence of SEQ ID NO: 6, SSRIYDFNSALISYYDMDV (SEQ ID NO: 11), or SSRIYDMISSLQPYYDMDV (SEQ ID NO:12), the LCDR1 comprises the amino acid sequence of SEQ ID NO: 8, the LCDR2 comprises the amino acid sequence of SEQ ID NO: 9; and the LCDR3 comprises the amino acid sequence of SEQ ID NO: In some embodiments, the anti-NGF antibody or fragment thereof comprises a VH having an amino acid sequence that is at least 80%, 85%, 90%, 95%, 97%, 99% or 100% identical to the amino acid sequence of SEQ ID NO: 3 or 94. In some embodiments, the anti-NGF antibody or fragment thereof comprises a VL having an amino acid sequence that is at least 80%, 85%, 90%, 95%, 97%, 99% or 100% identical to the amino acid sequence of SEQ ID NO: 7 or 95. In some embodiments, the anti-NGF antibody or fragment thereof is a full H₂L₂ antibody, an Fab, fragment, an Fab′ fragment, an F(ab)₂ fragment or a single chain Fv (scFv) fragment. In some embodiments, the anti-NGF antibody or fragment thereof is humanized, chimeric, primatized, or fully human. In some embodiments, the anti-NGF scFv fragment comprises, from N-terminus to C-terminus, a VH comprising an amino acid sequence that is at least 80%, 85%, 90%, 95%, 97%, 99% or 100% identical to the amino acid sequence of SEQ ID NO: 3, a 15-amino acid linker sequence (GGGGS)₃ (SEQ ID NO: 15), and a VL comprising an amino acid sequence that is at least 80%, 85%, 90%, 95%, 97%, 99% or 100% identical to the amino acid sequence of SEQ ID NO: 7. In some embodiments, the anti-NGF scFv fragment comprises, from N-terminus to C-terminus, a VH comprising an amino acid sequence that is at least 80%, 85%, 90%, 95%, 97%, 99% or 100% identical to the amino acid sequence of SEQ ID NO: 94, a 20-amino acid linker sequence (GGGGS)₄ (SEQ ID NO:19), and a VL comprising an amino acid sequence that is at least 80%, 85%, 90%, 95%, 97%, 99% or 100% identical to the amino acid sequence of SEQ ID NO: 95.

In some embodiments, the TNFR is TNFR-2. In some embodiments, the TNFR-2 fragment is fused to an immunoglobulin Fc domain. In some embodiments, the immunoglobulin Fc domain is a human IgG1 Fc domain. In some embodiments, the TNFα antagonist domain comprises an amino acid sequence that is at least 80%, 85%, 90%, 95%, 97%, 99% or 100% identical to the amino acid sequence set forth in SEQ ID NO: 13, or a functional fragment thereof.

In some embodiments, the binding molecule comprises a fusion protein that comprises the NGF antagonist fused to the TNFα antagonist through a linker. In some embodiments, the binding molecule comprises a homodimer of the fusion protein. In some embodiments, the binding molecule comprises a homodimer of a fusion polypeptide comprising, from N-terminus to C-terminus, a TNFα-binding fragment of TNFR-2 comprising an amino acid sequence that is at least 80%, 85%, 90%, 95%, 97%, 99% or 100% identical to a sequence corresponding to amino acids 1-235 of SEQ ID NO: 13, a human IgG1Fc domain, a 10 amino-acid linker sequence (GGGGS)₂(SEQ ID NO: 98), a VH comprising an amino acid sequence that is at least 80%, 85%, 90%, 95%, 97%, 99% or 100% identical to the amino acid sequence of SEQ ID NO 3 or 94, a 15-amino acid linker sequence (GGGGS)₃ (SEQ ID NO: 15), and a VL comprising an amino acid sequence that is at least 80%, 85%, 90%, 95%, 97%, 99% or 100% identical to the amino acid sequence of SEQ ID NO: 7 or 95. In some embodiments, the binding molecule comprises a homodimer of a fusion polypeptide comprising an amino acid sequence that is at least 80%, 85%, 90%, 95%, 97%, 99% or 100% identical to the amino acid sequence of SEQ ID NO: 14. In some embodiments, the binding molecule comprises a homodimer of a fusion polypeptide comprising, from N-terminus to C-terminus, a TNFα-binding 751(D fragment of TNFR-2 comprising the amino acid sequence of SEQ ID NO: 13, a 10-amino-acid linker sequence (GGGGS)₂ (SEQ ID NO: 98), a VH comprising the amino acid sequence of SEQ ID NO: 94, a 20-amino acid linker sequence (GGGGS)₄ (SEQ ID NO: 19), and a VL comprising the amino acid sequence of SEQ ID NO: 95. In some embodiments, the glycine residue at the amino acid position corresponding to position 102, 103, or 104 of SEQ ID NO: 7 is modified to a cysteine residue, and wherein the glycine residue at the amino acid position corresponding to position 44 of SEQ ID NO: 3 is modified to a cysteine residue. In some embodiments, the binding molecule comprises a homodimer of a fusion polypeptide comprising the amino acid sequence of SEQ ID NO: 17. In some embodiments, the binding molecule comprises a homodimer of a fusion polypeptide comprising an amino acid sequence that is at least 80%, 85%, 90%, 95% or 99% identical to the amino acid sequence of SEQ ID NO: 17.

The disclosure provides for a binding molecule for use in a method of reducing or preventing pain in a subject in need thereof, the method comprising administering to the subject a subcutaneous fixed dose of a binding molecule, wherein the binding molecule comprises an NGF antagonist domain and a TNFα antagonist domain, wherein the NGF antagonist domain is an anti-NGF antibody or an antigen-binding fragment thereof, wherein the TNFα antagonist domain comprises a soluble TNFα binding fragment of TNFR, and wherein the method reduces or prevents pain in the subject.

### BRIEF DESCRIPTION OF THE DRAWINGS

FIG. 1: Schematic representation of a TNFR2-Fc fusion protein (Panel A), and an exemplary multispecific binding molecule, TNFR2-Fc_VH #4, comprising a TNFR2-Fc domain fused to an anti-NGF scFv domain (panel B).

FIG. 2A shows the results of SEC-HPLC analysis of the levels of aggregate, monomer and protein fragmentation in a batch of purified TNFR2-Fc_VH #4.

FIG. 2B shows SDS-PAGE analysis of purified TNFR2-Fc_VH #4 and the purified TNFR2-Fc protein under reduced and non-reduced conditions. Gel loading order: 1. TNFR2-Fc_VH #4, 2. TNFR2-Fc_VL-VH (TNFR2-Fc fused to an anti-NGF scFv with reverse variable domain gene orientation), 3. TNFR2-Fc irrelevant scFv 1, 4. TNFR2-Fc, 5. TNFR2-Fc irrelevant scFv 2.

FIG. 3A shows the purity of TNFR2-Fc_VH #4 following Protein A column purification. FIG. 3B shows the purity of TNFR2-Fc_VH #4 following a second purification step on an SP sepharose column.

FIG. 4 shows a stability analysis of TNFR2-Fc_VH #4 using differential scanning calorimetry.

FIG. 5 shows binding of TNFR2-Fc_VH #4 to TNFα and NGF, both singly and together, as determined by ELISA. FIG. 5A shows binding to NGF, FIG. 5B shows binding to TNFα, and FIG. 5C shows simultaneous binding to TNFα and NGF.

FIG. 6 shows a sensorgram of a surface plasmon resonance binding assay for TNFR2-Fc_VH #4. Concurrent antigen binding of the TNFR2-Fc_VH #4 multispecific antibody was performed using BIAcore 2000. Simultaneous antigen binding was assessed by serially binding TNFα and NGF over TNFR2-Fc_VH #4 bound to the sensor surface. The first part of the sensorgram shows binding of saturating amounts of TNFα to the multispecific antibody, the second part of the sensorgram shows binding when a second antigen was applied, either TNFα again, which showed the surface was saturated, or an equimolar mixture of TNFα and NGF. An increase in resonance units equated to binding of the NGF to the multispecific molecule, and hence simultaneous antigen engagement. The assay was also performed with antigen addition in the reverse order confirming these data.

FIG. 7 shows the inhibition of NGF-mediated proliferation of TF-1 cells. A. NGF-mediated proliferation in the absence of added NGF antagonist. B. Inhibition of human NGF response by TNFR2-Fc_VH #4. C. Inhibition of murine NGF response by TNFR2-Fc_VH #4. Activity of NGF is normally represented as RLU−Relative luminescence Unit, and % of NGF mediated proliferation calculated as % response to NGF ligand alone using the following formula: 100*(well RLU−background RLU)/(Total RLU−background RLU), wherein background RLU=average of media controls, and Total RLU=average of ligand only controls. D. Inhibition of human NGF response by TNFR2-Fc_VarB and ndimab VarB. E. Inhibition of murine NGF response by TNFR2-Fc_VarB and ndimab VarB.

FIG. 8 shows the inhibition of TNFα induced Caspase 3 activity in U937 cells. A. TNFα induced Caspase 3 activity in U937 cells in the absence of added TNFα antagonist. B. Inhibition of TNFα induced Caspase 3 activity in U937 cells shown as percent of response in the absence of added antagonist. Activity of TNF is normally represented as RFU−Relative Florescence Unit, and % of TNF mediated caspase 3 release was calculated as % response to TNF ligand alone using the using the formula as described above in FIG. 7C: C. Similar results shown for a related molecule TNFR2-Fc_varB and ndimab VarB.

FIG. 9 shows the effect of combination treatment with etanercept and MEDI-578 on a partial sciatic nerve ligation-induced mechanical hyperalgesia. Results are shown as the ipsilateral/contralateral ratio. N=9-10 per group. Data was analyzed using a 2-way ANOVA analysis with time and treatment as dependent factors. Subsequent statistical significance was obtained using Boniferroni's Post Hoc test. ***p<0.001 to Op+CAT-251 control.

FIG. 10A shows the effect of TNFR2-Fc_VH #4 on partial sciatic nerve ligation-induced mechanical hyperalgesia. Results are shown as the ipsilateral/contralateral ratio. N=10 per group. Data was analyzed using a 2-way ANOVA analysis with time and treatment as dependent factors. Subsequent statistical significance was obtained using Boniferroni's Post Hoc test. ***p<0.001 vs bispecific isotype control. FIG. 10B shows similar results with a related molecule TNFR2-Fc_varB.

FIG. 11 shows the effect of co-administration of MEDI-578 and etanercept on pain reduction in a joint pain model of mechanical hypersensitivity. N=9-10 per group. Data was analyzed using a 2-way ANOVA analysis. Subsequent statistical significance was obtained using Boniferroni's Post Hoc test. *P>0.05; ***P<0.001 vs. CAT-251.

FIG. 12 shows the effect of TNFR2-Fc_VH #4 on pain reduction in a joint pain model of mechanical hypersensitivity. N=9-10 per group. Data was analyzed using a 2-way ANOVA analysis. Subsequent statistical significance was obtained using Boniferroni's Post Hoc test. ***P<0.001 vs. bispecific isotype control.

FIG. 13 shows the effects of five different doses of TNFR2-Fc_varB on CFA-induced hyperalgesia in a rat model.

FIG. 14: A heat map showing HTRF ratios from phospho-p38 reactions.

FIG. 15: Dose response curves showing the effect of TNFα, NGF, or a combination of TNFα and NGF on p38 phosphorylation.

FIG. 16: A heat map showing HTRF ratios from phospho-ERK reactions.

FIG. 17: Dose response curves showing the effect of TNFα, NGF, or a combination of TNFα and NGF on ERK phosphorylation.

FIG. 18A shows a simplified diagram of the interleaved Single Ascending Dose (SAD) and Multiple Ascending Dose (MAD) study. FIG. 18B shows in tabular form the study design for each cohort. “RoA” is route of administration, “IV” is intravenous, “SC” is subcutaneous. The predicted average percent NGF suppression is also provided.

FIG. 19A shows a graph in which the effect of a single intravenous dose of TNFR2-Fc_varB on average daily pain scored is plotted vs. time (days post-dose). The upper horizontal red line is the average daily pain score for all subjects pre-dose. The lower horizontal red line is the average daily pain score for all subjects receiving placebo. FIG. 19B is a table indicating the predicted mean NGF suppression percentage and the peak NRS change vs. placebo (PBO) at the listed doses.

FIG. 20A is a graph of baseline adjusted mean pain WOMAC after administration of TNFR2-Fc_varB. Subjects answer five questions that focus specifically on pain (while walking, stair climbing, nocturnal, at rest and weight bearing). Each question is given a score on a 5-point scale (0-4) with 0 being “none” and 4 being “Extremely.” The higher the score the worse the pain experienced carrying out that activity (or the greater the perceived functional deficit). Subjects answering all five pain questions can have a maximum score of 20, scaled down to 10 here to enable comparison with pain NRS scores. Subjects were requested to complete the questionnaire in clinic at baseline (1 day prior to dosing) and on days 8, 15, 22, 29, (and for cohorts 250 and 1000 μg/kg only days 43 and 56). FIG. 20B is a table providing p-values for the comparisons of the WOMAC scores of placebo vs. the different TNFR2-Fc_varB doses in the SAD study.

FIG. 21 is a table showing on the three statistically significant, single doses of TNFR2-Fc_varB, the measured % NGF suppression at peak and average across the 2 weeks post dose, and in parenthesis are the predicted NGF suppression levels. The peak WOMAC pain subscale change vs. placebo is also presented for each of these three doses. Note that peak effect corresponds with measured suppression of free NGF of 46-55% at doses of 50 and 250 μg/kg respectively.

FIG. 22 shows suppression of plasma free NGF as a result of administration of single doses of TNFR2-Fc_varB. In brief; blood samples were taken from each subject at the following timepoints; pre dose, 1, 8 and 24 hrs post dose, days 8, 15, 22, 29, (days 43 and 56 for the two highest doses only). Plasma samples were prepared and assayed using an Singulex, Erenna technology. Suppression of free NGF was calculated and the average suppression over the 14 day period, post dose, at each concentration calculated. Average suppression of free NGF over 14 days ranges from 0 (0.3 μg per kg) to −65% (1000 μg per kg).

FIG. 23 is a series of graphs plotting an increase in NGF levels for each subject in SAD cohorts 1-4 (0.3-50 μg/kg).

FIG. 24 is a graph plotting the percent mean change of CXCL-13 levels from baseline for each cohort vs. time.

FIG. 25 shows the geometric mean serum pharmacokinetic profiles of TNFR2-Fc_varB (denoted as MEDI7352) at single intravenous doses ranging from 0.3 to 1000 μg/kg and at single subcutaneous dose of 50 μg/kg. FIG. 25 displays the data on a logarithmic scale. For doses up to 50 μg/kg, samples were collected up to Day 29 post-dose. For doses of 250 and 1000 μg/kg, sampling was extended up to Day 43 and Day 56 post-dose. Data for 250 μg/kg are not shown beyond Day 29 because values for all subjects in the cohort were below the lower limit of quantification on Days 43 and 56. For 1000 μg/kg, values were above the lower limit of quantification for only 3 subjects at Day 43 and 1 subject at Day 56. LLOQ=Lower limit of quantification.

FIG. 26 shows the geometric mean serum pharmacokinetic profiles of TNFR2-Fc_varB (denoted as MEDI7352) at repeated intravenous doses ranging from 1 to 450 μg/kg. FIG. 26A presents the data on a linear scale. FIG. 26B displays the data on a logarithmic scale. Data for 1 μg/kg are not shown beyond Day 57 post-dose because values for all subjects in the cohort were below the lower limit of quantification on Days 64, 71 and 84. Data for 50 μg/kg are not shown for Day 84 because all subjects had concentrations below the lower limit of quantification. For 450 μg/kg, no concentration data are available beyond Day 57

FIG. 27 shows the maximum observed serum concentration of TNFR2-Fc_varB at Day 43 post-dose (Cₘₐₓ; top graph) and associated change in WOMAC pain score from baseline (bottom graph) after repeated intravenous doses of TNFR2-Fc_varB ranging from 1 to 450 μg/kg.

FIG. 28 shows pain levels after repeated doses of TNFR2-Fc_varB. FIG. 28A shows the change from baseline in NRS pain from Day 0-84 in patients who received placebo, 150 μg/kg or 450 μg/kg TNFR2-Fc_varB. FIG. 28B compares the effects of repeated doses of TNFR2-Fc_varB with 2.5 mg tanezumab, 5 mg tanezumab, 40 mg oxycodone, or placebo. FIG. 28C shows pain reduction, determined by change in the WOMAC pain subscale from baseline, induced by different doses of fasinumab, fulranumab, TNFR2-Fc_varB (denoted as MEDI7352) and tanezumab.

FIG. 29 shows the effect of ADA titer on TNFR2-Fc_varB (denoted as MEDI7352) concentration and pain relief determined by change in the WOMAC pain subscale (top graph) or NRS pain subscale (bottom graph).

FIG. 30 shows the geometric mean serum pharmacokinetic profile of TNFR2-Fc_varB at single intravenous doses ranging from 0.3 to 1000 μg/kg and repeated intravenous doses ranging from 1 to 450 μg/kg categorized by levels of ADA titer.

FIG. 31 is a scatter plot of TNFR2-Fc_varB clearance versus body weight after 4 twice-weekly doses. The legend indicates MAD cohort numbers and TNFR2-Fc_varB doses. Clearance data were obtained from non-compartmental analysis. The plot shows linear regression analysis (solid line) with 95% confidence limits (dashed lines). The p-value of 0.61 indicates that there is no significant association between clearance and weight.

FIG. 32 shows a simplified diagram of the subcutaneous fixed dose study.

### DETAILED DESCRIPTION

### Definitions

It is to be noted that the term “a” or “an” entity refers to one or more of that entity. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% above or below a given value.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

As used herein, the term “binding molecule” refers in its broadest sense to a molecule that specifically binds an antigenic determinant, e.g., antigen. Non-limiting example of an binding molecule include antibodies or fragment thereof, soluble receptor fusion proteins or fragment thereof, non-immunoglobulin scaffolds or fragments thereof, each retaining antigen specific binding. Exemplary soluble receptor fusion proteins and antibodies are provided below. In certain embodiments, the binding molecule could be engineered to comprise combinations of such antibodies or fragments thereof, soluble receptor fusion proteins or fragments thereof, and non-immunoglobulin-based scaffolds or fragment thereof.

The binding molecule, or any portion of the binding molecule that recognizes an antigen is referred to herein as a “binding domain.” Unless specifically referring to full-sized binding molecules such as naturally-occurring antibodies, the term “binding molecule” encompasses, without limitation, full-sized antibodies or other non-antibody binding molecules, as well as antigen-binding fragments, variants, analogs, or derivatives of such binding molecules, e.g., naturally occurring antibody or immunoglobulin molecules or engineered binding molecules or fragments that bind antigen in a manner similar to full-sized binding molecule.

In certain embodiments, the disclosure provides certain multi-specific binding molecules, e.g., bispecific, trispecific, tetraspecific, etc. binding molecules, or antigen-binding fragments, variants, or derivatives thereof. As used herein, a multi-specific binding molecule can include one or more antibody binding domains, one or more non-antibody binding domains, or a combination thereof.

The term “nerve growth factor” (“NGF”) also referred to in the literature as beta-nerve growth factor, as used herein refers to a secreted protein that functions in the growth and survival of various neurons. Human NGF is presented as Genbank Accession Number NP_002497.2, and is presented here as SEQ ID NO: 1. The term NGF as used herein is not limited to human NGF, and includes all species orthologs of human NGF. The term “NGF” encompasses the pro-form of NGF, pro-NGF, full-length NGF, as well as any form of NGF that results from processing within the cell. The term also encompasses naturally occurring variants of NGF, e.g., splice variants, allelic variants, and isoforms. NGF can bind to two receptors: the p75 neurotrophin receptor (p75(NTR)) and TrkA, a transmembrane tyrosine kinase. NGF is a well-validated target for pain being known to mediate sensitization of nociceptors.

NGF-mediated pain is particularly well suited to safe and effective treatment with binding molecules as set forth herein because NGF levels increase in the periphery in response to noxious stimuli and antibodies have low blood-brain barrier permeability. A number of anti-NGF antibodies and antigen-binding fragments thereof which can be used in the therapies and compositions described herein can be found in the literature, see, e.g., PCT Publication Nos. WO02/096458 and WO04/032870.

The term “MEDI-578” refers to an antibody that specifically binds NGF, which is the subject of International Appl. No. PCT/GB2006/000238 and U.S. Patent Appl. Pub. No. 2008/0107658 A1, both of which are incorporated by reference herein in their entirety. The MEDI-578 heavy and light chain sequences are shown in SEQ ID NOs: 3 and 7, respectively.

The term NGF-NG refers to an antibody that specifically binds NGF. The NGF-NG heavy and light chain sequences are shown in SEQ ID NOs: 24 and 26, respectively.

The term “tumor necrosis factor alpha” (“TNFα”), also referred to in the literature as cachectin, APC1 protein; tumor necrosis factor; TNF; or tumor necrosis factor ligand superfamily member 2, as used herein refers to the specific TNFα protein, and not the superfamily of TNF ligands. Human TNFα is presented as Genbank Accession Number NP_000585.2, and is presented as SEQ ID NO: 2. The term TNFα as used herein is not limited to human TNF, and includes all species orthologs of human TNFα. The term “TNFα” encompasses the pro-form of TNFα, pro-TNFα, full-length TNFα, as well as any form of TNFα that results from processing within the cell. The term also encompasses naturally occurring and non-naturally-occurring variants of TNFα, e.g., splice variants, allelic variants, and isoforms. TNFα can bind two receptors, TNFR1 (TNF receptor type 1; CD120a; p55/60) and TNFR2 (TNF receptor type 2; CD120b; p75/80). TNFα functions as a pro-inflammatory cytokine, e.g., functioning in neuroinflammation. For example, TNFα is thought to be functionally involved in the generation of neuropathic pain (Leung, L., and Cahill, CM., J. Neuroinflammation 7:27 (2010)).

An “isolated” binding molecule, polypeptide, antibody, polynucleotide, vector, host cell, or composition refers to a binding molecule, polypeptide, antibody, polynucleotide, vector, host cell, or composition that is in a non-naturally-occurring form. Isolated binding molecules, polypeptides, antibodies, polynucleotides, vectors, host cells or compositions include those which have been changed, adapted, combined, rearranged, engineered, or otherwise manipulated to a degree that they are no longer in the form in which they are found in nature. In some aspects a binding molecule, antibody, polynucleotide, vector, host cell, or composition that is isolated is “recombinant.”

As used herein, the terms “multifunctional polypeptide” and “bifunctional polypeptide” refer to a non-naturally-occurring binding molecule designed to target two or more antigens. An exemplary multifunctional polypeptide described herein is a multifunctional binding molecule comprising an anti-NGF antigen-binding fragment or antibody portion, and a soluble TNFR2 portion.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)₂, and Fv fragments), single chain Fv (scFv) mutants, multispecific antibodies such as bispecific, trispecific, tetraspecific, etc antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations.

In some embodiments, a “blocking” binding molecule, e.g., a blocking antibody or an “antagonist” binding molecule, such as for example, an antagonist antibody or fusion protein is one that inhibits or reduces biological activity of the antigen to which it binds, such as NGF or TNFα. In certain aspects blocking antibodies or antagonist binding molecules substantially or completely inhibit the biological activity of the antigen. For example, the biological activity can be reduced by 0.01%, 0.1%, 0.5%, 1%, 5%, 10%, 20%, 30%, 50%, 70%, 80%, 90%, 95%, or even 100%.

“Antagonists” and “antagonist domains” as used herein include polypeptides or other molecules that bind to their target (e.g., TNFα or NGF), thereby blocking or inhibiting the target from interacting with a receptor. NGF and/or TNFα antagonists thus include molecules that block or inhibit NGF interaction with trkA or p75 neurotrophin, or TNFα interaction with TNFR-1 or TNFR-2. NGF and/or TNFα antagonists also include molecules that reduce p38 phosphorylation and/or ERK phosphorylation. Exemplary antagonists include, but are not limited to anti-NGF antibodies or antigen-binding fragments thereof, and target-specific, soluble, non-signaling TNF-alpha receptor peptides (“decoy receptors,” or ligand-binding fragments thereof).

The term “antibody fragment” refers to a portion of an intact antibody and refers to the antigenic determining variable regions of an intact antibody. Examples of antibody fragments include, but are not limited to Fab, Fab′, F(ab′)₂, and Fv fragments, linear antibodies, single chain antibodies, and multispecific antibodies formed from antibody fragments. Antigen-binding fragments of non-antibody binding molecules, described elsewhere herein, are also provided by this disclosure.

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)₂, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, by hybridoma, phage selection, recombinant expression, and transgenic animals.

The term “humanized antibody” refers to forms of non-human (e.g., murine) antibodies that are specific immunoglobulin chains, chimeric immunoglobulins, or fragments thereof that contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, or hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the Fv framework region (FR or FW) residues of a human immunoglobulin are replaced with the corresponding residues in an antibody from a non-human species that has the desired specificity, affinity, and capability. The humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. No. 5,225,539 or 5,639,641.

A “variable region” of an antibody refers to the variable region of the antibody light chain or the variable region of the antibody heavy chain, either alone or in combination. The variable regions of the heavy and light chain each consist of four framework regions (FR or FW) connected by three complementarity-determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al (1997) J. Molec. Biol. 273:927-948)). In addition, combinations of these two approaches are sometimes used in the art to determine CDRs.

The Kabat numbering system is generally used when referring to a residue in the variable domain (approximately residues 1-107 of the light chain and residues 1-113 of the heavy chain) (e.g., Kabat et al., Sequences of Immunological Interest, 5th Ed. Public Health Service, National Institutes of Health, Bethesda, Md. (1991)).

The amino acid position numbering as in Kabat, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, MD. (1991). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g., residues 82a, 82b, and 82c, etc according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software. A comparison is provide in Table 1 below.

TABLE 1
Comparison of Antibody Numbering Systems

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The term “human antibody” means a native human antibody or an antibody having an amino acid sequence corresponding to a native human antibody, made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species. Multispecific binding molecules, e.g., including one or more antibody binding domains, one or more non-antibody binding domains, or a combination thereof, e.g., TNFα antagonists and/or NGF antagonists provided herein can comprise antibody constant regions (e.g., Fc regions) in which at least a fraction of one or more of the constant region domains has been deleted or otherwise altered so as to provide desired biochemical characteristics such as increased tumor localization or reduced serum half-life when compared with an antibody of approximately the same immunogenicity comprising a native or unaltered constant region. Modified constant regions provided herein can comprise alterations or modifications to one or more of the three heavy chain constant domains (CH1, CH2 or CH3) and/or to the light chain constant domain (CL). In some aspects, one or more constant domains can be partially or entirely deleted. In some aspects, the entire CH2 domain can be deleted (ΔCH2 constructs). See, e.g., Oganesyan V, et al., 2008 Acta Crystallogr D Biol Crystallogr. 64:700-4; Oganesyan V, et al., Mol Immunol. 46:1750-5; Dall'Acqua, W. F., et al., 2006. J. Biol. Chem. 281:23514-23524; and Dall'Acqua, et al., 2002. J. Immunol. 169:5171-5180.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation. An epitope as described herein need not be defined down to the specific amino acids that form the epitope. In some aspects an epitope can be identified by examination of binding to peptide subunits of a polypeptide antigen, or by examining binding competition to the antigen by a group of antigen-specific antibodies.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, bears, and so on.

The terms “composition” and “pharmaceutical composition” refer to a preparation which is in such form as to permit the biological activity of the active ingredient to be effective, and which contains no additional components which are unacceptably toxic to a subject to which the composition would be administered. Such compositions can be sterile.

As used herein, the terms “effective amount” and “therapeutically effective amount” refer to an amount of one or more therapeutic compositions effective to treat or control pain in a subject. The terms “treat pain”, “control pain” and grammatical equivalents are used herein to describe any beneficial or desirable effect in a subject in need of pain control. For example, an effective amount of one or more therapeutic compositions described herein can, e.g., prevent pain, maintain a tolerable level of pain, ameliorate pain, reduce pain, minimize pain, and/or eliminate pain in the subject. In particular, the terms “treat pain”, “control pain” and grammatical equivalents are used herein to describe the reduction of pain and/or the prevention of pain.

The term “administering” as used herein refers to administering to a subject one or more therapeutic compositions described herein, e.g., a bifunctional polypeptide comprising an NGF antagonist domain and a TNFα antagonist domain. The term “co-administering” refers to administering to a subject two or more therapeutic compositions. As used herein, co-administering includes, but does not require that the two or more therapeutic compositions be administered to the subject simultaneously. The two or more therapeutic compositions can be administered to the subject sequentially, e.g., thirty minutes apart, one hour apart, two hours apart, three hours apart, four hours apart, or five or more hours apart. The sequence and timing of a co-administration as described herein can be fixed, or can be varied based on the judgment of a healthcare professional.

The terms “polynucleotide” and “nucleic acid” refer to a polymeric compound comprised of covalently linked nucleotide residues. Polynucleotides can be DNA, cDNA, RNA, single stranded, or double stranded, vectors, plasmids, phage, or viruses.

The term “vector” means a construct, which is capable of delivering, and expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The terms “polypeptide,” “peptide,” and “protein” are used interchangeably herein to refer to polymers of amino acids of any length. The polymer can be linear or branched, it can comprise modified amino acids, and non-amino acids can interrupt it. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

A “conservative amino acid substitution” is one in which one amino acid residue is replaced with another amino acid residue having a similar side chain Families of amino acid residues having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain aspects, conservative substitutions in the sequences of polypeptides and antibodies provided herein do not abrogate the binding or other functional activity of the polypeptide containing the amino acid sequence. Methods of identifying nucleotide and amino acid conservative substitutions which do not affect function are well-known in the art (see, e.g., Brummell et al., Biochem. 32: 1180-1 187 (1993); Kobayashi et al. Protein Eng. 12:879-884 (1999); and Burks et al. Proc. Natl. Acad. Sci. USA 94:.412-417 (1997)).

### Binding Molecule Comprising an NGF Antagonist Domain and a TNFα Antagonist Domain

This disclosure provides a bifunctional polypeptide comprising an NGF antagonist domain and a TNFα antagonist domain for use in any of the methods disclosed herein (e.g., according to any of the dosage regimens disclosed herein). In certain aspects, administration of an effective amount of a bifunctional polypeptide provided herein can control pain, in a subject in need thereof, more effectively than an equivalent amount of the NGF antagonist or the TNFα antagonist administered alone. Bifunctional polypeptides provided herein can include the NGF antagonist domain and the TNFα antagonist domain in any order, structure, or conformation. Any suitable NGF antagonists or TNFα antagonists can be part of a bifunctional polypeptide provided herein. Exemplary NGF antagonists and TNFα antagonists are described in this disclosure.

In certain aspects, the NGF antagonist is an anti-NGF antibody, or antigen-binding fragment thereof. In certain aspects, an anti-NGF antagonist, e.g., an antagonist antibody or fragment thereof for use in a bifunctional molecule provided herein, e.g., a multispecific binding molecule, can preferentially block NGF binding to TrkA over NGF binding to p75NRT.

Exemplary antibodies or fragments thereof for use in bifunctional polypeptides, e.g., multispecific binding molecules disclosed herein are available in U.S. Appl. Publication No. 2008/0107658, which is incorporated herein by reference in its entirety. In certain aspects, the anti-NGF antibody or fragment thereof binds to the same epitope as, can competitively inhibit, or can bind to NGF with a greater affinity than the anti-NGF antibody MEDI-578. In certain embodiments, the anti-NGF antibody or fragment thereof binds human NGF and/or rat NGF with an affinity of or less than 1, 0.8, 0.7, 0.6, 0.4, 0.3 or 0.2 nM. For example, the anti-NGF antibody or fragment thereof may bind human NGF with an affinity of about 0.2-0.8, 0.2-0.7, 0.2-06, 0.2-0.5, and/or 0.25-nM and rat NGF with an affinity of about 0.2-0.9, 0.2-0.8, and/or 0.25-0.70 nM.

In certain aspects, the anti-NGF antibody or fragment thereof is MEDI-578. MEDI-578 is disclosed in U.S. Appl. Publication No. 2008/0107658 as clone 1252A5. In other aspects, the anti-NGF antibody or fragment thereof is tanezumab (RN-624), a humanized anti-NGF mAb (Pfizer; described in Kivitz et al., (2013) PAIN, 154, 9, 1603-161), fulranumab, a fully human anti-NGF mAb (Amgen; described in Sanga et al., PAIN, Volume 154, Issue 10, October 2013, Pages 1910-1919); REGN475/SAR164877, a fully human anti-NGF mAb (Regeneron/Sanafi-Aventis); ABT-110 (PG110), a humanized anti-NGF mAb (Abbott Laboratories); fasinumab, a human anti-NGF mAb (Regeneron, disclosed in U.S. Appl. Publication No. 2009/0041717 as clone REGN475. An anti-NGF antibody or fragment thereof included in a bifunctional polypeptide, e.g., multispecific binding molecule provided herein, can be, e.g., humanized, chimeric, primatized, or fully human.

In certain aspects, the anti-NGF antibody or fragment thereof comprises an antibody VH domain comprising the HCDR1, HCDR2, and HCDR3 domains of MEDI-578, variants of the MEDI-578 heavy chain CDRs with up to one, two, three, four, five, or more amino acid substitutions, e.g., conservative amino acid substitutions. For example, the anti-NGF antibody or fragment thereof can comprise an HCDR1 with the exact amino acid sequence of SEQ ID NO: 4 or with the amino acid sequence of SEQ ID NO: 4 with one or more, e.g., one, two, three, four, five, or more amino acid substitutions. Similarly, the anti-NGF antibody or fragment thereof can comprise an HCDR2 with the exact amino acid sequence of SEQ ID NO: 5 or with the amino acid sequence of SEQ ID NO: 5 with one or more, e.g., one, two, three, four, five, or more amino acid substitutions. Likewise, the anti-NGF antibody or fragment thereof can comprise an HCDR3 with the exact amino acid sequence of SEQ ID NO: 6 or with the amino acid sequence of SEQ ID NO: 6 with one or more, e.g., one, two, three, four, five, or more amino acid substitutions. In certain aspects, the HCDR3 can comprise the amino acid sequence SSRIYDFNSALISYYDMDV (SEQ ID NO: 11), or SSRIYDMISSLQPYYDMDV (SEQ ID NO: 12).

In certain aspects, the anti-NGF antibody or fragment thereof comprises an antibody VL domain comprising the LCDR1, LCDR2, and LCDR3 domains of MEDI-578, variants of the MEDI-578 light chain CDRs with up to one, two, three, four, five, or more amino acid substitutions, e.g., conservative amino acid substitutions. In certain aspects, the anti-NGF antibody or fragment thereof can comprise an LCDR1 with the exact amino acid sequence of SEQ ID NO: 8 or with the amino acid sequence of SEQ ID NO: 8 with one or more, e.g., one, two, three, four, five, or more amino acid substitutions. Similarly, the anti-NGF antibody or fragment thereof can comprise an LCDR2 with the exact amino acid sequence of SEQ ID NO: 9 or with the amino acid sequence of SEQ ID NO: 9 with one or more, e.g., one, two, three, four, five, or more amino acid substitutions. Likewise, the anti-NGF antibody or fragment thereof can comprise an LCDR3 with the exact amino acid sequence of SEQ ID NO: 10 or with the amino acid sequence of SEQ ID NO: 10 with one or more, e.g., one, two, three, four, five, or more amino acid substitutions.

In certain aspects, the anti-NGF antibody or fragment thereof comprises an antibody VH domain comprising a VH amino acid sequence at least 80%, 85%, 90%, 95%, 96%, 97%, 98%, or 99% identical to the amino acid sequence of SEQ ID NO: 3. In some aspects the anti-NGF antibody or fragment thereof comprises an antibody VH domain comprising the VH amino acid sequence of SEQ ID NO: 3.

In certain aspects, the anti-NGF antibody or fragment thereof comprises an antibody VL domain comprising a VL amino acid sequence at least 80%, 85%, 90%, 95%, 96%, 97%, 98%, or 99% identical to the amino acid sequence of SEQ ID NO: 7. In some aspects the anti-NGF antibody or fragment thereof comprises an antibody VL domain comprising the VL amino acid sequence of SEQ ID NO: 7.

In certain aspects, the anti-NGF antibody or fragment thereof comprises an antibody VH domain comprising a VH amino acid sequence at least 80%, 85%, 90%, 95%, 96%, 97%, 98%, or 99% identical to the amino acid sequence of SEQ ID NO: 94. In some aspects the anti-NGF antibody or fragment thereof comprises an antibody VH domain comprising the VH amino acid sequence of SEQ ID NO: 94.

In certain aspects, the anti-NGF antibody or fragment thereof comprises an antibody VL domain comprising a VL amino acid sequence at least 80%, 85%, 90%, 95%, 96%, 97%, 98%, or 99% identical to the amino acid sequence of SEQ ID NO: 95. In some aspects the anti-NGF antibody or fragment thereof comprises an antibody VL domain comprising the VL amino acid sequence of SEQ ID NO: 95.

In certain aspects, the anti-NGF antibody or fragment thereof comprises an antibody VH domain comprising the HCDR1, HCDR2, and HCDR3 domains of any one of SEQ ID NOs: 30, 32, 34, 36, 38, 40, 42, 44, 46, 48, 50, 52, 54, 56, 58, 60, 62, 64, 66, 68, 70, 72, 74, 76, 78, 80, 82, 84, 86 and 96, or variants thereof with up to one, two, three, four, five, or more amino acid substitutions, e.g., conservative amino acid substitutions.

In certain aspects, the anti-NGF antibody or fragment thereof comprises an antibody VL domain comprising the LCDR1, LCDR2, and LCDR3 domains of any one of SEQ ID NOs: 31, 33, 35, 37, 39, 41, 43, 45, 47, 49, 51, 53, 55, 57, 59, 61, 63, 65, 67, 69, 71, 73, 75, 77, 79, 81, 83, 85, 87 and 97, or variants thereof with up to one, two, three, four, five, or more amino acid substitutions, e.g., conservative amino acid substitutions.

In certain aspects, the anti-NGF antibody or fragment thereof comprises an antibody VH domain comprising a VH amino acid sequence at least 80%, 85%, 90%, 95%, 96%, 97%, 98%, or 99% identical to the amino acid sequence of any one of SEQ ID NOs: 30, 32, 34, 36, 38, 40, 42, 44, 46, 48, 50, 52, 54, 56, 58, 60, 62, 64, 66, 68, 70, 72, 74, 76, 78, 80, 82, 84, 86 and 96. In some aspects the anti-NGF antibody or fragment thereof comprises an antibody VH domain comprising the VH amino acid sequence of any one of SEQ ID NOs: 30, 32, 34, 36, 38, 40, 42, 44, 46, 48, 50, 52, 54, 56, 58, 60, 62, 64, 66, 68, 70, 72, 74, 76, 78, 80, 82, 84, 86 and 96.

In certain aspects, the anti-NGF antibody or fragment thereof comprises an antibody VL domain comprising a VL amino acid sequence at least 80%, 85%, 90%, 95%, 96%, 97%, 98%, or 99% identical to the amino acid sequence of any one of SEQ ID NOs: 31, 33, 35, 37, 39, 41, 43, 45, 47, 49, 51, 53, 55, 57, 59, 61, 63, 65, 67, 69, 71, 73, 75, 77, 79, 81, 83, 85, 87 and 97. In some aspects the anti-NGF antibody or fragment thereof comprises an antibody VL domain comprising the VL amino acid sequence of any one of SEQ ID NOs: 31, 33, 35, 37, 39, 41, 43, 45, 47, 49, 51, 53, 55, 57, 59, 61, 63, 67, 69, 71, 73, 75, 77, 79, 81, 83, 85, 87 and 97.

In certain aspects, the anti-NGF antibody or fragment thereof comprises an antibody VH domain comprising the HCDR1, HCDR2, and HCDR3 domains of NGF-NG, variants of the NGF-NG heavy chain CDRs with up to one, two, three, four, five, or more amino acid substitutions, e.g., conservative amino acid substitutions. For example, the anti-NGF antibody or fragment thereof can comprise an HCDR1 with the exact amino acid sequence of SEQ ID NO: 88 or with the amino acid sequence of SEQ ID NO: 88 with one or more, e.g., one, two, three, four, five, or more amino acid substitutions. Similarly, the anti-NGF antibody or fragment thereof can comprise an HCDR2 with the exact amino acid sequence of SEQ ID NO: 89 or with the amino acid sequence of SEQ ID NO: 89 with one or more, e.g., one, two, three, four, five, or more amino acid substitutions. Likewise, the anti-NGF antibody or fragment thereof can comprise an HCDR3 with the exact amino acid sequence of SEQ ID NO: 90 or with the amino acid sequence of SEQ ID NO: 90 with one or more, e.g., one, two, three, four, five, or more amino acid substitutions.

In certain aspects, the anti-NGF antibody or fragment thereof comprises an antibody VL domain comprising the LCDR1, LCDR2, and LCDR3 domains of NGF-NG, variants of the NGF-NG light chain CDRs with up to one, two, three, four, five, or more amino acid substitutions, e.g., conservative amino acid substitutions. In certain aspects, the anti-NGF antibody or fragment thereof can comprise an LCDR1 with the exact amino acid sequence of SEQ ID NO: 91 or with the amino acid sequence of SEQ ID NO: 91 with one or more, e.g., one, two, three, four, five, or more amino acid substitutions. Similarly, the anti-NGF antibody or fragment thereof can comprise an LCDR2 with the exact amino acid sequence of SEQ ID NO: 92 or with the amino acid sequence of SEQ ID NO: 92 with one or more, e.g., one, two, three, four, five, or more amino acid substitutions. Likewise, the anti-NGF antibody or fragment thereof can comprise an LCDR3 with the exact amino acid sequence of SEQ ID NO: 93 or with the amino acid sequence of SEQ ID NO: 93 with one or more, e.g., one, two, three, four, five, or more amino acid substitutions.

In certain aspects, the anti-NGF antibody or fragment thereof comprises an antibody VH domain comprising a VH amino acid sequence at least 80%, 85%, 90%, 95%, 96%, 97%, 98%, or 99% identical to the amino acid sequence of SEQ ID NO: 24. In some aspects the anti-NGF antibody or fragment thereof comprises an antibody VH domain comprising the VH amino acid sequence of SEQ ID NO: 24.

In certain aspects, the anti-NGF antibody or fragment thereof comprises an antibody VL domain comprising a VL amino acid sequence at least 80%, 85%, 90%, 95%, 96%, 97%, 98%, or 99% identical to the amino acid sequence of SEQ ID NO: 26. In some aspects the anti-NGF antibody or fragment thereof comprises an antibody VL domain comprising the VL amino acid sequence of SEQ ID NO: 26.

A multifunctional polypeptide, e.g., multispecific binding molecule as provided by this disclosure can comprise a complete anti-NGF antibody, i.e., an antibody comprising two complete heavy chains and two complete light chains in an H₂L₂ format. Where the anti-NGF antibody is a complete antibody, one or more TNFα antagonist domains can be fused to the N-terminus or C-terminus of one or more heavy chains of the anti-NGF antibody or to the N-terminus or C-terminus of one or more light chains of the anti-NGF antibody. Alternatively, a multifunctional polypeptide, e.g., multispecific binding molecule as provided by this disclosure can comprise an antigen-binding fragment of an anti-NGF antibody. In certain aspects an anti-NGF antibody fragment can comprise any portion of the antibody's constant domains or can comprise only the variable domains. Exemplary anti-NGF antibody fragments for inclusion in a bifunctional polypeptide, e.g., multispecific binding molecule, include, but are not limited to Fab fragments, Fab′ fragments, F(ab)₂ fragments or single chain Fv (scFv) fragments.

In certain exemplary compositions provided herein, the anti-NGF antibody is a scFv fragment, e.g. an scFv fragment of MEDI-578, or an NGF-binding variant thereof. In certain exemplary compositions provided herein, the anti-NGF antibody is a scFv fragment, e.g. an scFv fragment of NGF-NG, or an NGF-binding variant thereof. An anti-NGF scFv polypeptide can comprise the VH and VL domains in any order, either N-VH-VL-C, or N-VL-VH-C. ScFv molecules are typically engineered such that the VH and VL domains are connected via a flexible linker. Exemplary scFv structures, including various linkers can be found in Dimasi, N., et al., J Mol Biol. 393:672-92 (2009), and in PCT Publication No. WO 2013/070565, both of which are incorporated herein by reference in their entireties. As is understood by persons of ordinary skill in the art, scFv antibody fragments can have reduced stability relative to the variable domains existing in a standard Fab conformation. In some aspects the scFv can be structurally stabilized by introducing stabilizing mutations or by introducing interchain disulfide bond(s) (e.g., SS-stabilized). However, stabilizing mutations and/or an introduced interchain disulfide bond is not required and, in certain aspects, is not present. A number of art-recognized methods are available to stabilize scFv polypeptides.

Linkers can be used to join domains/regions of bifunctional polypeptides provided herein. Linkers can be used to connect the NGF antagonist domain and the TNFα antagonist domain of a bifunctional molecule, and can also be used to interconnect the variable heavy and light chains of an scFv. An exemplary, non-limiting example of a linker is a polypeptide chain comprising at least 4 residues. Portions of such linkers can be flexible, hydrophilic and have little or no secondary structure of their own (linker portions or flexible linker portions). Linkers of at least 4 amino acids can be used to join domains and/or regions that are positioned near to one another after a bifunctional polypeptide molecule has assembled. Longer linkers can also be used. Thus, linkers can be about 1, 2, 3, 4, 5, 6, 7, 8, 9, 10, 11, 12, 13, 14, 15, 16, 17, 18, 19, 20, 25, 30, 35, 40, 50, residues. Linkers can also be, for example, from about 100-175 residues. When multiple linkers are used to interconnect portions of a bifunctional polypeptide molecule, the linkers can be the same or different (e.g., the same or different length and/or amino acid sequence).

The linker(s) in a bifunctional polypeptide molecule facilitate formation of the desired structure. Linkers can comprise (Gly-Ser)ₙ residues (where n is an integer of at least one, two and up to, e.g., 3, 4, 5, 6, 10, 20, 50, 100, or more), with some Glu or Lys residues dispersed throughout to increase solubility. Alternatively, certain linkers do not comprise any Serine residues, e.g., where the linker is subject to O-linked glycosyation. In some aspects, linkers can contain cysteine residues, for example, if dimerization of linkers is used to bring the domains of a bifunctional polypeptide into their properly folded configuration. In some aspects, a bifunctional polypeptide can comprise at least one, two, three, four, or more polypeptide linkers that join domains of the polypeptide.

In some aspects, a polypeptide linker can comprise 1-50 residues, 1-25 residues, residues, or 30-50 residues. In some aspects, the polypeptide linker can comprise a portion of an Fc moiety. For example, in some aspects, the polypeptide linker can comprise a portion of immunoglobulin hinge domain of an IgG1, IgG2, IgG3, and/or IgG4 antibody or a variant thereof.

In some aspects, a polypeptide linker can comprise or consist of a gly-ser linker. As used herein, the term “gly-ser linker” refers to a peptide that consists of glycine and serine residues. An exemplary gly-ser linker comprises an amino acid sequence of the formula (Gly 4 Ser)n, where n is an integer of at least one, two and up to, e.g., 3, 4, 5, 6, 20, 50, 100, or more. In some aspects, a polypeptide linker can comprise at least a portion of a hinge region (e.g., derived from an IgG1, IgG2, IgG3, or IgG4 molecule) and a series of gly-ser amino acid residues (e.g., a gly-ser linker such as (Gly 4 Ser)n).

When a multifunctional polypeptide, e.g., a multispecific binding molecule, comprises an scFv, a flexible linker can connect the heavy and light chains of the scFv. This flexible linker generally does not include a hinge portion, but rather, is a gly-ser linker or other flexible linker. The length and amino acid sequence of a flexible linker interconnecting domains of an scFv can be readily selected and optimized.

In certain aspects, a multifunctional polypeptide, e.g., a multispecific binding molecule, can comprise an anti-NGF scFv fragment which comprises, from N-terminus to C-terminus, a VH, a 15-amino acid linker sequence (GGGGS)₃, and a VL. In certain embodiments, the linker joining the VH and VL of the scFv is a 20 amino acid linker sequence (GGGGS)₄. In certain aspects the VH comprises the amino acid sequence of SEQ ID NO 3. In certain aspects the VL comprises the amino acid sequence of SEQ ID NO: 7. In certain embodiments, the VH comprises the amino acid sequence of any one of SEQ ID NOs: 24, 30, 32, 34, 36, 38, 40, 42, 44, 46, 48, 50, 52, 54, 56, 58, 60, 62, 64, 66, 68, 70, 72, 74, 76, 78, 80, 82, 84, 86, 94 and 96. In certain embodiments, the VL comprises the amino acid sequence of any one of SEQ ID NOs: 26, 31, 33, 35, 37, 39, 41, 43, 45, 47, 49, 51, 53, 55, 57, 59, 61, 63, 65, 67, 69, 71, 73, 75, 77, 79, 81, 83, 85, 87, 95 and 97. In certain aspects, the VH domain comprises an amino acid sequence at least 80%, 85%, 90%, 95%, 96%, 97%, 98%, or 99% identical to the amino acid sequence of any one of SEQ ID NOs: 3, 24, 30, 32, 34, 36, 38, 40, 42, 44, 46, 48, 50, 52, 54, 56, 58, 60, 62, 64, 66, 68, 70, 72, 74, 76, 78, 80, 82, 84, 86, 94 and 96. In certain aspects, the VL domain comprises an amino acid sequence at least 80%, 85%, 90%, 95%, 96%, 97%, 98%, or 99% identical to the amino acid sequence of any one of SEQ ID NOs: 7, 26, 31, 33, 35, 37, 39, 41, 43, 45, 47, 49, 51, 53, 55, 57, 59, 61, 63, 65, 67, 69, 71, 73, 75, 77, 79, 81, 83, 85, 87, 95 and 97.

In other aspects, the stability of the polypeptide can be improved by addition of an inter-chain disulphide bond between the VH domain and the VL domain by modifying certain residues within the VH and VL domain to cysteine residues. See for example, Michaelson, J. S., et al. (2009) MAbs 1, 128-41; Brinkmann, U., et al., (1993) Proc Natl Acad Sci USA 90, 7538-42; Young, N. M., et al., (1995) FEBS Lett 377, 135-9. For example, the glycine residue at positions 102, 103 or 104 of the VL (e.g., SEQ ID NO: 7) can be modified to a cysteine residue and the glycine residue at position 44 of the VH (e.g., SEQ ID NO: 3) can be modified to a cysteine residue. In some embodiments, the glycine residue at the amino acid position corresponding to position 102, 103, or 104 of SEQ ID NO: 7 is modified to a cysteine residue. In some embodiments, the glycine residue at the amino acid position corresponding to position 44 of SEQ ID NO: 3 is modified to a cysteine residue.

A multifunctional polypeptide, e.g., a multispecific binding molecule as provided herein includes a TNFα antagonist domain. In certain aspects, a TNFα antagonist domain can inhibit the binding of TNFα to a TNF receptor (TNFR) on the surface of cells, thereby blocking TNF activity.

In certain aspects, the TNFα antagonist is a TNFα-binding soluble fragment of a TNF receptor, e.g., TNFR-1 or TNFR-2, or a variant thereof or a soluble fragment thereof. In certain aspects, the soluble fragment of TNFR-1 is a 55 kD fragment. In certain embodiments, the soluble fragment of TNFR-2 is a 751(D fragment. In certain aspects the TNF receptor fragment is fused to a heterologous polypeptide, e.g., an immunoglobulin Fc fragment, e.g., an IgG1 Fc domain. In certain aspects, the TNFα antagonist comprises an amino acid set forth in SEQ ID NO: 13, or a TNFα-binding fragment thereof. The TNFR-2 portion comprises amino acids 1 to 235 of SEQ ID NO: 13. In certain aspects, a variant of a TNFα-binding soluble fragment of TNFR-2 comprises an amino acid sequence at least 80%, 85%, 90%, 95%, 96%, 97%, 98%, 99%, or 100% identical to amino acids 1 to 235 of SEQ ID NO: 13. In certain aspects, a variant of a TNFα-binding soluble fragment of TNFR-2 comprises amino acids 1 to 235 of SEQ ID NO: 13, except for, e.g., 1, 2, 3, 4, 5, 10, 20, 20, 40, or 50 amino acid insertions, substitutions, or deletions. The IgG1 Fc portion comprises amino acids 236 to 467 of SEQ ID NO: 13. In certain aspects, the TNFα-binding soluble fragment of TNFR-2 can be fused to an Fc portion of any human or non-human antibody, or to any other protein or non-protein substance that would provide stability, e.g., albumin or polyethylene glycol. In certain aspects, a variant of a TNFα-binding soluble fragment of TNFR-2 comprises an amino acid sequence at least 80%, 85%, 90%, 95%, 96%, 97%, 98%, 99%, or 100% identical to amino acids 236 to 467 of SEQ ID NO: 13. In certain aspects, a variant of a TNFα-binding soluble fragment of TNFR-2 comprises amino acids 236 to 467 of SEQ ID NO: 13, except for, e.g., 1, 2, 3, 4, 5, 10, 20, 20, 40, or 50 amino acid insertions, substitutions, or deletions. In certain aspects, a variant of a TNFα-binding soluble fragment of TNFR-2 comprises an amino acid sequence at least 80%, 85%, 90%, 95%, 96%, 97%, 98%, 99%, or 100% identical to SEQ ID NO: 13. In certain aspects, a variant of a TNFα-binding soluble fragment of TNFR-2 comprises SEQ ID NO: 13, except for, e.g., 1, 2, 3, 4, 5, 10, 20, 20, 40, or 50 amino acid insertions, substitutions, or deletions.

In certain aspects, TNFα-binding soluble fragment of TNFR-2 is a single-chain fusion protein. In certain aspects the TNFα-binding soluble fragment of TNFR-2 is a dimer of two fusion proteins, associated, e.g., through disulfide bonds between the two Fc domains.

A multifunctional polypeptide, e.g., a multispecific binding molecule, as provided herein can have a variety of different structures and conformations. In one aspect, a multifunctional polypeptide as provided herein comprises a fusion protein where the NGF antagonist domain, as described above, is fused to the TNFα antagonist domain, as described above, through a flexible linker. Examples of linkers are described elsewhere herein. In certain aspects, the multifunctional polypeptide comprises a homodimer of the fusion protein.

In an exemplary aspect, a multifunctional polypeptide is provided in which the NGF antagonist is an anti-NGF scFv domain derived, e.g., from MEDI-578 and the TNFα antagonist is a soluble, TNFα-binding fragment of TNFR-2 fused at its carboxy-terminus to an immunoglobulin Fc domain. The anti-NGF scFv can be, in some aspects, fused to the carboxy-terminus of the immunoglobulin Fc domain via a linker. In certain aspects, monomers of this multifunctional polypeptide form a homodimer with each subunit comprising, from N-terminus to C-terminus, a TNFα-binding 75 kD fragment of TNFR-2, a human IgG1Fc domain, a 10-amino-acid linker (GGGGS)₂ (SEQ ID NO: 98), an anti-NGF VH comprising the amino acid sequence of SEQ ID NO 3, a 15-amino acid linker sequence (GGGGS)₃ (SEQ ID NO: 15), and an anti-NGF VL comprising the amino acid sequence of SEQ ID NO: 7. In one aspect, the multifunctional polypeptide is TNFR2-Fc_VH #4, which comprises a homodimer of a fusion polypeptide comprising the amino acid sequence of SEQ ID NO: 14. In some aspects, the multifunctional polypeptide comprises a homodimer of a fusion polypeptide comprising an amino acid sequence that is at least 80%, 85%, 90%, 95%, 96%, 97%, 98%, or 99% identical to SEQ ID NO: 14.

In another exemplary aspect, the multifunctional polypeptide comprises, from N-terminus to C-terminus, a TNFα-binding 75 kD fragment of TNFR-2, a human IgG1Fc domain, a 10-amino-acid linker (GGGGS)₂ (SEQ ID NO: 98), an anti-NGF VH comprising the amino acid sequence of SEQ ID NO 94, a 20-amino acid linker sequence (GGGGS)₄ (SEQ ID NO: 19), and an anti-NGF VL comprising the amino acid sequence of SEQ ID NO: 95. In some embodiments, the binding molecule comprises, from N-terminus to C-terminus, a TNFα-binding 751(D fragment of TNFR-2 comprising an amino acid sequence that is at least 80%, 85%, 90%, 95%, 96%, 97%, 98%, or 99% identical to the SEQ ID NO: 13, a human IgG1Fc domain, a 10-amino-acid linker (GGGGS)₂ (SEQ ID NO: 98), an anti-NGF VH comprising an amino acid sequence that is at least 80%, 85%, 90%, 95%, 96%, 97%, 98%, or 99% identical to the amino acid sequence of SEQ ID NO 94, a 20-amino acid linker sequence (GGGGS)₄ (SEQ ID NO: 19), and an anti-NGF VL comprising an amino acid sequence that is at least 80%, 85%, 90%, 95%, 96%, 97%, 98%, or 99% identical to the amino acid sequence of SEQ ID NO: In some aspect, the multifunctional polypeptide is TNFR2-Fc_varB, which comprises a homodimer of a fusion polypeptide comprising the amino acid sequence of SEQ ID NO: 17. In some aspects, the multifunctional polypeptide comprises a homodimer of a fusion polypeptide comprising an amino acid sequence that is at least 80%, 85%, 90%, 95%, 96%, 97%, 98%, 99% identical to SEQ ID NO: 17.

### Polynucleotides, Vectors, and Host Cells

This disclosure provides nucleic acid molecules comprising polynucleotides that encode any of the binding molecules disclosed herein for use in any of the methods disclosed herein (e.g., and of the dosage regimens disclosed herein). This disclosure further provides nucleic acid molecules comprising polynucleotides that encode individual polypeptides comprising, respectively, an NGF antagonist and a TNFα antagonist. In certain aspects such polynucleotides encode a peptide domain that specifically binds NGF or a fragment thereof, and also binds TNFα or a fragment thereof. For example, this disclosure provides a polynucleotide that encodes a polypeptide domain comprising an anti-NGF antibody or an antigen-binding fragment thereof, and a polypeptide domain comprising a TNFα antagonist, such as an anti-TNFα antibody or antigen-binding fragment thereof, or a soluble TNFα-binding portion of a TNF receptor, e.g., TNFR2. Polynucleotides can be in the form of RNA or in the form of DNA. DNA includes cDNA, genomic DNA, and synthetic DNA; and can be double-stranded or single-stranded, and if single stranded can be the coding strand or non-coding (anti-sense) strand.

In some embodiments, the isolated polynucleotide that encodes a multifunctional polypeptide described herein comprises the nucleotide sequence of SEQ ID NO: 16, 18 or 99, or fragments thereof, or a sequence at least 80%, 85%, 90%, 95%, 96%, 97%, 98%, or 99% identical to SEQ ID NO: 16, 18 or 99, or fragments thereof.

The isolated polypeptides described herein can be produced by any suitable method known in the art. Such methods range from direct protein synthetic methods to constructing a DNA sequence encoding isolated polypeptide sequences and expressing those sequences in a suitable transformed host. In some aspects, a DNA sequence is constructed using recombinant technology by isolating or synthesizing a DNA sequence encoding a multifunctional polypeptide comprising an NGF antagonist domain and a TNFα antagonist domain, or individual polypeptides comprising an NGF antagonist domain and a TNFα antagonist domain, respectively. Accordingly, this disclosure provides an isolated polynucleotide that encodes a bifunctional polypeptide comprising an NGF antagonist domain and a TNFα antagonist domain as described in detail above. Further provided are isolated polynucleotides that encode individual polypeptides that comprise, respectively, an NGF antagonist domain and a TNFα antagonist domain.

In some aspects a DNA sequence encoding a multifunctional polypeptide, e.g., a multispecific binding molecule of interest or individual polypeptides comprising an NGF antagonist domain and a TNFα antagonist domain, respectively can be constructed by chemical synthesis using an oligonucleotide synthesizer. Such oligonucleotides can be designed based on the amino acid sequence of the desired multifunctional polypeptide and selecting those codons that are favored in the host cell in which the recombinant polypeptide of interest will be produced. Standard methods can be applied to synthesize an isolated polynucleotide sequence encoding a multifunctional polypeptide of interest. For example, a complete amino acid sequence can be used to construct a back-translated gene. Further, a DNA oligomer containing a nucleotide sequence coding for the particular multifunctional polypeptide or individual polypeptides can be synthesized. For example, several small oligonucleotides coding for portions of the desired polypeptide can be synthesized and then ligated. The individual oligonucleotides typically contain 5′ or 3′ overhangs for complementary assembly.

In certain aspects, polynucleotides provided herein can comprise the coding sequence for the mature polypeptide fused in the same reading frame to a marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used.

Polynucleotides provided herein can further contain alterations in the coding regions, non-coding regions, or both. In some aspects the polynucleotide variants contain alterations that produce silent substitutions, additions, or deletions, but do not alter the properties or activities of the encoded polypeptide. In some aspects, nucleotide variants are produced by silent substitutions due to the degeneracy of the genetic code. Polynucleotide variants can be produced for a variety of reasons, e.g., to optimize codon expression for a particular host (change codons in the human mRNA to those preferred by a bacterial host such as E. coli).

Vectors and cells comprising the polynucleotides described herein are also provided. Once assembled (by synthesis, site-directed mutagenesis or another method), the polynucleotide sequences encoding a particular isolated polypeptide of interest can be inserted into an expression vector and operatively linked to an expression control sequence appropriate for expression of the protein in a desired host. This disclosure provides such vectors. Nucleotide sequencing, restriction mapping, and expression of a biologically active polypeptide in a suitable host can confirm proper assembly. As is well known in the art, in order to obtain high expression levels of a transfected gene in a host, the gene must be operatively linked to transcriptional and translational expression control sequences that are functional in the chosen expression host.

In certain aspects, recombinant expression vectors can be used to amplify and express DNA encoding multifunctional polypeptides, e.g., multispecific binding molecules, comprising an NGF antagonist domain and a TNFα antagonist domain, or individual polypeptides comprising an NGF antagonist domain and a TNFα antagonist domain, respectively. Recombinant expression vectors are replicable DNA constructs that have synthetic or cDNA-derived DNA fragments encoding a multifunctional polypeptide or individual polypeptides comprising an NGF antagonist domain and a TNFα antagonist domain, respectively, operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail below. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

The choice of expression control sequence and expression vector will depend upon the choice of host. A wide variety of expression host/vector combinations can be employed. Useful expression vectors for eukaryotic hosts include, for example, vectors comprising expression control sequences from SV40, bovine papilloma virus, adenovirus and cytomegalovirus. Useful expression vectors for bacterial hosts include known bacterial plasmids, such as plasmids from E. coli, including pCR 1, pBR322, pMB9 and their derivatives, wider host range plasmids, such as M13 and filamentous single-stranded DNA phages.

This disclosure further provides host cells comprising polynucleotides encoding the polypeptides provided herein. Suitable host cells for expression of the polypeptides provided herein include prokaryotes, yeast, insect or higher eukaryotic cells under the control of appropriate promoters. Prokaryotes include gram negative or gram-positive organisms, for example E. coli or bacilli. Higher eukaryotic cells include established cell lines of mammalian origin as described below. Cell-free translation systems can also be employed. Appropriate cloning and expression vectors for use with bacterial, fungal, yeast, and mammalian cellular hosts are described by Pouwels et al. (Cloning Vectors: A Laboratory Manual, Elsevier, N.Y., 1985), the relevant disclosure of which is hereby incorporated by reference. Additional information regarding methods of protein production, including antibody production, can be found, e.g., in U.S. Patent Publication No. 2008/0187954, U.S. Pat. Nos. 6,413,746 and 6,660,501, and International Patent Publication No. WO 04009823, each of which is hereby incorporated by reference herein in its entirety.

Various mammalian or insect cell culture systems can also be advantageously employed to express recombinant protein. Expression of recombinant proteins in mammalian cells can be performed because such proteins are generally correctly folded, appropriately modified and completely functional. Examples of suitable mammalian host cell lines include HEK-293 and HEK-293T, the COS-7 lines of monkey kidney cells, described by Gluzman (Cell 23:175, 1981), and other cell lines including, for example, L cells, C127, 3T3, Chinese hamster ovary (CHO), HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

This disclosure further provides a method of producing the multifunctional polypeptide as described herein, or for producing individual polypeptides comprising, respectively an NGF antagonist, and a TNFα antagonist. The method entails culturing a host cell as described above under conditions promoting expression of the multifunctional polypeptide or individual polypeptides, and recovering the multifunctional polypeptide or individual polypeptides.

For long-term, high-yield production of recombinant proteins, stable expression is appropriate. For example, cell lines which stably express the multifunctional polypeptide may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may be used to engineer cell lines which express the multifunctional polypeptide.

In certain embodiments, multifunctional polypeptides presented herein are expressed in a cell line with transient expression of the multifunctional polypeptide. Transient transfection is a process in which the nucleic acid introduced into a cell does not integrate into the genome or chromosomal DNA of that cell but is maintained as an extrachromosomal element, e.g. as an episome, in the cell. Transcription processes of the nucleic acid of the episome are not affected and a protein encoded by the nucleic acid of the episome is produced.

The cell line, either stable or transiently transfected, is maintained in cell culture medium and conditions known in the art resulting in the expression and production of polypeptides. In certain embodiments, the mammalian cell culture media is based on commercially available media formulations, including, for example, DMEM or Ham's F12. In some embodiments, the cell culture media is modified to support increases in both cell growth and biologic protein expression. As used herein, the terms “cell culture medium,” “culture medium,” and “medium formulation” refer to a nutritive solution for the maintenance, growth, propagation, or expansion of cells in an artificial in vitro environment outside of a multicellular organism or tissue. Cell culture medium may be optimized for a specific cell culture use, including, for example, cell culture growth medium which is formulated to promote cellular growth, or cell culture production medium which is formulated to promote recombinant protein production. The terms nutrient, ingredient, and component may be used interchangeably to refer to the constituents that make up a cell culture medium.

In various embodiments, the cell lines are maintained using a fed batch method. As used herein, “fed batch method,” refers to a method by which a fed batch cell culture is supplied with additional nutrients after first being incubated with a basal medium. For example, a fed batch method may comprise adding supplemental media according to a determined feeding schedule within a given time period. Thus, a “fed batch cell culture” refers to a cell culture where the cells, typically mammalian, and culture medium are supplied to the culturing vessel initially and additional culture nutrients are fed, continuously or in discrete increments, to the culture during culturing, with or without periodic cell and/or product harvest before termination of culture.

In some embodiments, the cell culture medium comprises a basal medium and at least one hydrolysate, e.g., soy-based, hydrolysate, a yeast-based hydrolysate, or a combination of the two types of hydrolysates resulting in a modified basal medium. The additional nutrients may sometimes include only a basal medium, such as a concentrated basal medium, or may include only hydrolysates, or concentrated hydrolysates. Suitable basal media include, but are not limited to Dulbecco's Modified Eagle's Medium (DMEM), DME/F12, Minimal Essential Medium (MEM), Basal Medium Eagle (BME), RPMI 1640, F-10, F-12, .alpha.-Minimal Essential Medium (.alpha.-MEM), Glasgow's Minimal Essential Medium (G-MEM), PF CHO (see, e.g., CHO protein free medium (Sigma) or EX-CELL™ 325 PF CHO Serum-Free Medium for CHO Cells Protein-Free (SAFC Bioscience), and Iscove's Modified Dulbecco's Medium. Other examples of basal media which may be used in the technology herein include BME Basal Medium (Gibco-Invitrogen; Dulbecco's Modified Eagle Medium (DMEM, powder) (Gibco-Invitrogen (#31600)).

In certain embodiments, the basal medium may be is serum-free, meaning that the medium contains no serum (e.g., fetal bovine serum (PBS), horse serum, goat serum, or any other animal-derived serum known to one skilled in the art) or animal protein free media or chemically defined media.

The basal medium may be modified in order to remove certain non-nutritional components found in standard basal medium, such as various inorganic and organic buffers, surfactant(s), and sodium chloride. Removing such components from basal cell medium allows an increased concentration of the remaining nutritional components, and may improve overall cell growth and protein expression. In addition, omitted components may be added back into the cell culture medium containing the modified basal cell medium according to the requirements of the cell culture conditions. In certain embodiments, the cell culture medium contains a modified basal cell medium, and at least one of the following nutrients, an iron source, a recombinant growth factor; a buffer; a surfactant; an osmolarity regulator; an energy source; and non-animal hydrolysates. In addition, the modified basal cell medium may optionally contain amino acids, vitamins, or a combination of both amino acids and vitamins In some embodiments, the modified basal medium further contains glutamine, e.g, L-glutamine, and/or methotrexate.

In some embodiments, protein production is conducted in large quantity by a bioreactor process using fed-batch, batch, perfusion or continuous feed bioreactor methods known in the art. Large-scale bioreactors have at least 50 L liters of capacity, sometimes about more than 500 liters or 1,000 to 100,000 liters of capacity. These bioreactors may use agitator impellers to distribute oxygen and nutrients. Small scale bioreactors refers generally to cell culturing in no more than approximately 100 liters in volumetric capacity, and can range from about 1 liter to about 100 liters. Alternatively, single-use bioreactors (SUB) may be used for either large-scale or small scale culturing.

Temperature, pH, agitation, aeration and inoculum density may vary depending upon the host cells used and the recombinant protein to be expressed. For example, a recombinant protein cell culture may be maintained at a temperature between 30 and 45 degrees Celsius. The pH of the culture medium may be monitored during the culture process such that the pH stays at an optimum level, which may be for certain host cells, within a pH range of 6.0 to 8.0. An impellor driven mixing may be used for such culture methods for agitation. The rotational speed of the impellor may be approximately 50 to 200 cm/sec tip speed, but other airlift or other mixing/aeration systems known in the art may be used, depending on the type of host cell being cultured. Sufficient aeration is provided to maintain a dissolved oxygen concentration of approximately 20% to 80% air saturation in the culture, again, depending upon the selected host cell being cultured. Alternatively, a bioreactor may sparge air or oxygen directly into the culture medium. Other methods of oxygen supply exist, including bubble-free aeration systems employing hollow fiber membrane aerators.

### Protein Purification

In some embodiments, the disclosure provides for methods of purifying any of the binding molecules disclosed herein for use in any of the methods disclosed herein (e.g., any of the dosage regimens disclosed herein). The proteins produced by a transformed host as described above can be purified according to any suitable method. Such standard methods include chromatography (e.g., ion exchange, affinity and sizing column chromatography), centrifugation, differential solubility, or by any other standard technique for protein purification. Affinity tags such as hexahistidine, maltose binding domain, influenza coat sequence and glutathione-S-transferase can be attached to the protein to allow easy purification by passage over an appropriate affinity column. Isolated proteins can also be physically characterized using such techniques as proteolysis, nuclear magnetic resonance and x-ray crystallography.

For example, supernatants from systems that secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify an NGF-binding agent. Some or all of the foregoing purification steps, in various combinations, can also be employed to provide a homogeneous recombinant protein.

Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying recombinant polypeptides also include, for example, those described in U.S. Patent Publication No. 2008/0312425, 2008/0177048, and 2009/0187005, each of which is hereby incorporated by reference herein in its entirety.

### Methods of Use and Pharmaceutical Compositions

This disclosure provides methods for controlling or treating pain in a subject, such as reducing and/or preventing pain in a subject, comprising administering a therapeutically effective amount of a TNFα and NGF antagonist multifunctional polypeptide, e.g., a multispecific binding molecule, as provided herein or comprising co-administration of a TNFα antagonist and an NGF antagonist. In certain aspects, the subject is a human.

This disclosure further provides pharmaceutical compositions comprising any of the binding molecules described herein. In certain aspects, the pharmaceutical compositions further comprise a pharmaceutically acceptable vehicle. These pharmaceutical compositions are useful in treating, such as reducing or preventing, pain, e.g., neuropathic and inflammatory (e.g., osteo or rheumatoid-arthritic) pain.

The multifunctional polypeptides and compositions comprising an NGF antagonist and a TNFα antagonist provided herein can be useful in a variety of applications including, but not limited to, the control or treatment (e.g., reduction and/or prevention) of pain, e.g., neuropathic pain. The methods of use may be in vitro, ex vivo, or in vivo methods.

In certain aspects, the disease, disorder, or condition treated with the NGF-binding agent (e.g., an antibody or polypeptide) is associated with pain. In certain aspects, the pain is associated with chronic nociceptive pain, chronic lower back pain, neuropathic pain, cancer pain, postherpetic neuralgia (PHN) pain, or visceral pain conditions. In certain aspects, the pain is associated with joint inflammation, such as inflammation of a knee or hip.

This disclosure provides a method for controlling, such as reducing or preventing, pain in a subject, comprising administering to a subject in need of pain control an effective amount of a nerve growth factor (NGF) antagonist and a tumor necrosis factor (TNFα) antagonist, wherein the administration can control (e.g., reduce or prevent) pain in the subject more effectively than an equivalent amount of the NGF antagonist or the TNFα antagonist administered alone.

By controlling pain “more effectively” than the components administered alone it is meant that the combination treatment is more effective at controlling pain than equivalent amounts of either the NGF antagonist or the TNFα antagonist administered individually. In certain aspects, and as described in more detail below, the method of controlling (e.g., reducing or preventing) pain provided herein can provide synergistic efficacy, e.g., the effect of the administration of both the NGF antagonist and the TNFα antagonist can provide more than an additive effect, or can be effective where neither the NGF antagonist nor the TNFα antagonist are effective individually. In certain aspects the combination can allow for dose sparing, e.g., the effective dosages of the individual components when co-administered can be less than the effective doses of either component individually.

In certain aspects, the method of controlling (e.g., reducing or preventing) pain provided herein is at least 5%, 10%, 20%, 30%, 40%, 50%, 60%, 70% 80%, 90%, or 100% more effective at controlling (e.g., reducing or preventing) pain in the subject than an equivalent amount of the NGF antagonist or the TNFα antagonist administered alone. In certain aspects, dosages of the individual NGF antagonist or the TNFα antagonist co-administered to the subject or the dose of the relative dose of the NGF antagonist or the TNFα antagonist provided upon administration of a bifunctional polypeptide provided herein can be lower, e.g., 5%, 10%, 20%, 30%, 40%, 50% 60%, 70%, 80% or 90% lower than the dosages necessary for the components administered alone.

In some embodiments, the disclosure provides for administering any of the binding molecules disclosed herein to a subject at a specific dosage regimen. In some embodiments, any of the binding molecules disclosed herein is administered to any of the subjects disclosed herein at a dose of 0.04-0.25 mg/kg. In some embodiments, any of the binding molecules disclosed herein is administered to any of the subjects disclosed herein at a dose of 0.04-0.15 mg/kg. In some embodiments, any of the binding molecules disclosed herein is administered to any of the subjects disclosed herein at a dose of 0.04-mg/kg. In some embodiments, any of the binding molecules disclosed herein is administered to any of the subjects disclosed herein at a dose of 0.04-0.075 mg/kg. In some embodiments, any of the binding molecules disclosed herein is administered to any of the subjects disclosed herein at a dose of 0.04-0.06 mg/kg. In some embodiments, any of the binding molecules disclosed herein is administered to any of the subjects disclosed herein at a dose of about 0.05 mg/kg. In some embodiments, any of the binding molecules disclosed herein is administered to any of the subjects disclosed herein at a dose of about 0.1 mg/kg. In some embodiments, any of the binding molecules disclosed herein is administered to any of the subjects disclosed herein at a dose of about 0.15 mg/kg. In some embodiments, any of the binding molecules disclosed herein is administered to any of the subjects disclosed herein at a dose of about 0.2 mg/kg. In some embodiments, any of the binding molecules disclosed herein is administered intravenously. In some embodiments, any of the binding molecules disclosed herein is administered subcutaneously.

In some embodiments, the disclosure provides for a method for treating (e.g., reducing or preventing) pain in a subject in need thereof, comprising intravenously administering to the subject 0.04-0.275 mg/kg of any of the binding molecules disclosed herein. In some embodiments, the method comprises intravenously administering to the subject 0.04-0.25 mg/kg of the binding molecule. In some embodiments, the method comprises intravenously administering to the subject 0.04-0.2 mg/kg of the binding molecule. In some embodiments, the method comprises intravenously administering to the subject 0.04-0.15 mg/kg of the binding molecule. In some embodiments, the method comprises intravenously administering to the subject 0.04-0.1 mg/kg of the binding molecule. In some embodiments, the method comprises intravenously administering to the subject 0.04-0.08 mg/kg of the binding molecule. In some embodiments, the method comprises intravenously administering to the subject 0.1-0.275 mg/kg of the binding molecule. In some embodiments, the method comprises intravenously administering to the subject 0.1-0.25 mg/kg of the binding molecule. In some embodiments, the method comprises intravenously administering to the subject 0.1-0.2 mg/kg of the binding molecule. In some embodiments, the method comprises intravenously administering to the subject 0.15-0.25 mg/kg of the binding molecule. In some embodiments, the method comprises intravenously administering to the subject about 0.05 mg/kg of the binding molecule. In some embodiments, the method comprises intravenously administering to the subject about 0.1 mg/kg of the binding molecule. In some embodiments, the method comprises intravenously administering to the subject about 0.15 mg/kg of the binding molecule. In some embodiments, the method comprises intravenously administering to the subject about 0.2 mg/kg of the binding molecule.

In some embodiments, the disclosure provides for a method for treating (e.g. reducing or preventing) pain in a subject in need thereof, comprising subcutaneously administering to the subject 0.1-1.2 mg/kg of any of the binding molecules disclosed herein. In some embodiments, the disclosure provides for a method for treating (e.g., reducing or preventing) pain in a subject in need thereof, comprising subcutaneously administering to the subject 0.1-1.0 mg/kg of any of the binding molecules disclosed herein. In some embodiments, the disclosure provides for a method for treating (e.g., reducing or preventing) pain in a subject in need thereof, comprising subcutaneously administering to the subject 0.1-0.8 mg/kg of any of the binding molecules disclosed herein. In some embodiments, the disclosure provides for a method for treating (e.g., reducing or preventing) pain in a subject in need thereof, comprising subcutaneously administering to the subject 0.1-0.6 mg/kg of any of the binding molecules disclosed herein. In some embodiments, the disclosure provides for a method for treating (e.g., reducing or preventing) pain in a subject in need thereof, comprising subcutaneously administering to the subject 0.1-0.4 mg/kg of any of the binding molecules disclosed herein. In some embodiments, the disclosure provides for a method for treating (e.g., reducing or preventing) pain in a subject in need thereof, comprising subcutaneously administering to the subject 0.1-0.25 mg/kg of any of the binding molecules disclosed herein. In some embodiments, the disclosure provides for a method for treating (e.g., reducing or preventing) pain in a subject in need thereof, comprising subcutaneously administering to the subject 0.4-1.0 mg/kg of any of the binding molecules disclosed herein. In some embodiments, the disclosure provides for a method for treating (e.g., reducing or preventing) pain in a subject in need thereof, comprising subcutaneously administering to the subject 0.6-1.0 mg/kg of any of the binding molecules disclosed herein. In some embodiments, the disclosure provides for a method for treating (e.g., reducing or preventing) pain in a subject in need thereof, comprising subcutaneously administering to the subject 0.8-1.0 mg/kg of any of the binding molecules disclosed herein. In some embodiments, the disclosure provides for a method for treating (e.g., reducing or preventing) pain in a subject in need thereof, comprising subcutaneously administering to the subject 0.8-1.2 mg/kg of any of the binding molecules disclosed herein. In some embodiments, the disclosure provides for a method for treating (e.g., reducing or preventing) pain in a subject in need thereof, comprising subcutaneously administering to the subject about 0.2 mg/kg of any of the binding molecules disclosed herein. In some embodiments, the disclosure provides for a method for treating (e.g., reducing or preventing) pain in a subject in need thereof, comprising subcutaneously administering to the subject about 0.4 mg/kg of any of the binding molecules disclosed herein. In some embodiments, the disclosure provides for a method for treating (e.g., reducing or preventing) pain in a subject in need thereof, comprising subcutaneously administering to the subject about 0.6 mg/kg of any of the binding molecules disclosed herein. In some embodiments, the disclosure provides for a method for treating (e.g., reducing or preventing) pain in a subject in need thereof, comprising subcutaneously administering to the subject about 0.8 mg/kg of any of the binding molecules disclosed herein. In some embodiments, the disclosure provides for a method for treating (e.g., reducing or preventing) pain in a subject in need thereof, comprising subcutaneously administering to the subject about 1 mg/kg of any of the binding molecules disclosed herein.

In some embodiments, the disclosure provides a method of treating, e.g. reducing or preventing, pain in a subject in need thereof by administering any of the binding molecules disclosed herein to the subject at a fixed dosage regimen. As used herein, a fixed dosage regimen means that the dosage given to each subject is fixed, and is not dependent on the weight or other characteristics of the subject. In some embodiments, any of the binding molecules disclosed herein is administered to any of the subjects disclosed herein at a fixed dose of 5-200 mg. In some embodiments, any of the binding molecules disclosed herein is administered to any of the subjected disclosed herein at a dose of 7.5-150 mg. In some embodiments, any of the binding molecules disclosed herein is administered to any of the subjects disclosed herein at a dose of 25-150 mg. In some embodiments, any of the binding molecules disclosed herein is administered to any of the subjects disclosed herein at a dose of 75-150 mg. In some embodiments, any of the binding molecules disclosed herein is administered to any of the subjects disclosed herein at a dose of 5, 7.5, 25, 75, 150 or 200 mg. In some embodiments, any of the binding molecules disclosed herein is administered to any of the subjects disclosed herein at a dose of 7.5, 25, 75 or 150. In some embodiments, any of the binding molecules disclosed herein is administered to any of the subjects disclosed herein at a dose of 5 mg. In some embodiments, any of the binding molecules disclosed herein is administered to any of the subjects disclosed herein at a dose of 7.5 mg. In some embodiments, any of the binding molecules disclosed herein is administered to any of the subjects disclosed herein at a dose of 25 mg. In some embodiments, any of the binding molecules disclosed herein is administered to any of the subjects disclosed herein at a dose of 75 mg. In some embodiments, any of the binding molecules disclosed herein is administered to any of the subjects disclosed herein at a dose of 150 mg. In some embodiments, any of the binding molecules disclosed herein is administered to any of the subjects disclosed herein at a dose of 200 mg. In some embodiments, any of the binding molecules disclosed herein is administered to any of the subjects disclosed herein at a fixed dose equivalent to an intravenous dose of the binding molecule. In some embodiments, a fixed dose equivalent to an intravenous dose is a fixed dose which provides substantially the same, or the same, serum pharmacokinetic profile as the intravenous dose. In some embodiments, a fixed dose equivalent to an intravenous dose is a fixed dose which provides substantially the same, or the same, geometric mean area under the curve in a pharmacokinetic profile plot as the intravenous dose. In some embodiments, any of the binding molecules disclosed herein is administered to any of the subjects disclosed herein at a fixed dose equivalent to an intravenous fixed dose of the binding molecule. In some embodiments, any of the binding molecules disclosed herein is administered to any of the subjects disclosed herein at a fixed dose equivalent to a fixed intravenous dose of 30 mg of the binding molecule.

In some embodiments, any of the binding molecules disclosed herein is administered intravenously. In some embodiments, any of the binding molecules disclosed herein is administered intravenously to any of the subjects disclosed herein. In some embodiments, any of the binding molecules disclosed herein is administered at a fixed dose intravenously.

In some embodiments, any of the binding molecules disclosed herein is administered subcutaneously. In some embodiments, any of the binding molecules disclosed herein is administered subcutaneously to any of the subjects disclosed herein. In some embodiments, any of the binding molecules disclosed herein is administered at a fixed dose subcutaneously. In some embodiments, any of the binding molecules disclosed herein is administered subcutaneously at any of the fixed doses disclosed herein.

In some embodiments, the disclosure provides for a method for treating, e.g. preventing or reducing pain, in a subject in need thereof, comprising administering to the subject a subcutaneous fixed dose of any of the binding molecules disclosed herein. In some embodiments, the method comprises subcutaneously administering a fixed dose of 5-200 mg of any of the binding molecules disclosed herein. In some embodiments, the method comprises subcutaneously administering a fixed dose of 7.5-150 mg of any of the binding molecules disclosed herein. In some embodiments, the method comprises subcutaneously administering a fixed dose of 25-150 mg of any of the binding molecules disclosed herein. In some embodiments, the method comprises subcutaneously administering a fixed dose of 75-150 mg of any of the binding molecules disclosed herein. In some embodiments, the method comprises subcutaneously administering a fixed dose of 5, 7.5, 25, 75, 150, or 200 mg of any of the binding molecules disclosed herein. In some embodiments, the method comprises subcutaneously administering a fixed dose of 7.5, 25, 75 or 150 mg of any of the binding molecules disclosed herein In some embodiments, any of the binding molecules disclosed herein is administered subcutaneously to any of the subjects disclosed herein at a dose of 5 mg. In some embodiments, any of the binding molecules disclosed herein is administered subcutaneously to any of the subjects disclosed herein at a dose of 7.5 mg. In some embodiments, any of the binding molecules disclosed herein is administered subcutaneously to any of the subjects disclosed herein at a dose of 25 mg. In some embodiments, any of the binding molecules disclosed herein is administered subcutaneously to any of the subjects disclosed herein at a dose of 75 mg. In some embodiments, any of the binding molecules disclosed herein is administered subcutaneously to any of the subjects disclosed herein at a dose of 150 mg. In some embodiments, any of the binding molecules disclosed herein is administered subcutaneously to any of the subjects disclosed herein at a dose of 200 mg. In some embodiments, the method comprises subcutaneously administering a fixed dose equivalent to an intravenous fixed dose of the binding molecule. In some embodiments, the method comprises subcutaneously administering a fixed dose equivalent to a fixed intravenous dose of 30 mg of the binding molecule.

In some embodiments, the method of treating (e.g. preventing or reducing) pain comprises administering any of the binding molecules disclosed herein according to a dosage schedule. In some embodiments, the binding molecule is administered to the subject once. In some embodiments, the binding molecule is administered to the subject multiple times. In some embodiments, a fixed dose of the binding molecule is administered to the subject multiple times. In some embodiments, the same fixed dose of the binding molecule is administered to the subject multiple times. In some embodiments, the binding molecule (e.g. a fixed dose of the binding molecule) is administered to the subject at least once a week, no more than once a week, at least once every two weeks, no more than once every two weeks, at least once every three weeks, no more than once every three weeks, at least once a month, no more than once a month, at least twice a month, no more than twice a month, at least three times a month, no more than three times a month, at least once every six weeks, or no more than once every six weeks. In some embodiments, the binding molecule (e.g. a fixed dose of the binding molecule) is administered to the subject at least once every two weeks. In some embodiments, the binding molecule (e.g. a fixed dose of the binding molecule) is administered to the subject no more than once every two weeks. In some embodiments, the binding molecule (e.g. a fixed dose of the binding molecule) is administered to the subject once every two weeks. In some embodiments, the binding molecule (e.g. a fixed dose of the binding molecule) is administered to the subject at least once every three weeks. In some embodiments, the binding molecule is administered to the subject no more than once every three weeks. In some embodiments, the binding molecule (e.g. a fixed dose of the binding molecule) is administered to the subject once every three weeks. In some embodiments, the binding molecule (e.g. a fixed dose of the binding molecule) is administered to the subject at least once a month. In some embodiments, the binding molecule (e.g. a fixed dose of the binding molecule) is administered to the subject no more than once a month. In some embodiments, the binding molecule (e.g. a fixed dose of the binding molecule) is administered to the subject once a month.

The disclosure provides for a method of treating, e.g., preventing or reducing, pain wherein the dosage schedule for administering any of the binding molecules disclosed herein continues for a set period. For example, a fixed dose of the binding molecule may be administered at least once every 2 weeks for at least 12 weeks. In some embodiments, the binding molecule is administered for at least 4 weeks, at least 8 weeks, at least 12 weeks, or at least 16 weeks. In some embodiments, the binding molecule is administered for at least 4 weeks. In some embodiments, the binding molecule is administered for at least 8 weeks. In some embodiments, the binding molecule is administered for at least 12 weeks. In some embodiments, the binding molecule is administered for at least 16 weeks. In some embodiments, the binding molecule is administered for 12 weeks. In some embodiments, the binding molecule is administered at least once every 2 weeks for at least 12 weeks. In some embodiments, the binding molecule is administered once every 2 weeks for at least 12 weeks. In some embodiments, the binding molecule is administered once every 2 weeks for 12 weeks.

Any of the binding molecules disclosed herein may be used for the reduction or prevention of pain in combination with an additional pain treatment. The additional pain treatment may be administered concurrently with any of the binding molecules disclosed herein or independently of any of the binding molecules disclosed herein. Therefore, the disclosure provides for a method of reducing or preventing pain in a subject in need thereof, comprising administering any of the binding molecules disclosed herein and further comprising administering an additional pain treatment. In some embodiments, the method of preventing or reducing pain further comprises administering an NSAID to the subject. In some embodiments, the method further comprises administering an opioid to the subject. In some embodiments, the method further comprises administering acetaminophen to the subject. In some embodiments, the method further comprises administering paracetamol to the subject. In some embodiments, the method further comprises administering a COX-2 inhibitor to the subject.

The subject in need of pain treatment may have been suffering from pain for some time before being administered any of the binding molecules disclosed herein. In some embodiments of the method of preventing or reducing pain, the subject has suffered the pain for 1 month or longer prior to administration of the binding molecule. In some embodiments, the subject has suffered the pain for 3 months or longer prior to administration with the binding molecule. In some embodiments, the subject has suffered the pain for 6 months or longer prior to administration with the binding molecule.

Before initiation of treatment with any of the binding molecules disclosed herein, the subject may have already been administered with an alternative treatment for pain. In some embodiments, the method of preventing or reducing pain comprises administering the subject with an alternative treatment for pain prior to administration of any of the binding molecules disclosed herein and determining that the alternative treatment for pain does not reduce or prevent pain in the subject and/or that the subject is intolerant to the alternative treatment for pain. In some embodiments, the alternative treatment for pain is a NSAID, strong opioid, weak opioid, COX-2 inhibitor, acetaminophen or a combination thereof. In some embodiments, the method comprises the following steps prior to administration of the binding molecule to the subject: a. administering to the subject a NSAID, strong opioid, weak opioid, COX-2 inhibitor, acetaminophen or a combination thereof, and b. determining i) that the NSAID, strong opioid, weak opioid, COX-2 inhibitor, acetaminophen or a combination thereof does not reduce or prevent pain in the subject, and/or ii) determining that the subject is intolerant to the NSAID, strong opioid, weak opioid, COX-2 inhibitor, acetaminophen or a combination thereof. In some embodiments, the NSAID, strong opioid, weak opioid, COX-2 inhibitor, acetaminophen or a combination thereof is administered for at least 1 week, at least 2 weeks, at least 3 weeks, or at least 4 weeks. In some embodiments, the NSAID, strong opioid, weak opioid, COX-inhibitor, acetaminophen or a combination thereof is administered for at least 2 weeks. In some embodiments, the NSAID, strong opioid, weak opioid, COX-2 inhibitor, acetaminophen or a combination thereof has been administered to the subject for at least 1 week, at least 2 weeks, at least 3 weeks, or at least 4 weeks prior to administration with any of the binding molecules disclosed herein In some embodiments, the NSAID, strong opioid, weak opioid, COX-2 inhibitor, acetaminophen or a combination thereof has been administered to the subject for at least 2 weeks prior to administration with any of the binding molecules disclosed herein. In some embodiments, the subject is intolerant to NSAIDs, strong opioid, weak opioids, COX-2 inhibitors, acetaminophen or a combination thereof.

Before initiation of treatment with any of the binding molecules disclosed herein, the subject may be tested for the presence of an infection. In some embodiments, the method of preventing or reducing pain comprises testing the subject for SARS-CoV2 infection prior to administration with any of the binding molecules disclosed herein. In some embodiments, the method comprises testing the subject for SARS-CoV2 infection prior to administration of a fixed dose of the binding molecule to the subject. In some embodiments, testing the subject for SARS-CoV2 infection comprises testing the subject for SARS-CoV2 genetic material prior to administration of a fixed dose of the binding molecule to the subject. In some embodiments, the subject is not infected with SARS-CoV2 at baseline. The subject may negative for SARS-CoV2 ribonucleic acid (RNA) at baseline as tested by PCR. The subject may show no clinical signs or symptoms consistent with COVID-19 infection or an acute viral respiratory illness, e.g. fever, cough, dyspnea, sore throat and/or loss of taste/smell. The subject may be negative for SARS-CoV2 may be negative for COVID-19 antibodies.

The invention provides methods for controlling or treating (e.g. reducing or preventing) pain. In certain aspects, the pain is selected from chronic nociceptive pain, chronic lower back pain, neuropathic pain, cancer pain, postherpetic neuralgia (PHN) pain, or visceral pain conditions. In certain aspects, the pain is associated with joint inflammation, such as inflammation of a knee or hip.

The binding molecules disclosed herein may be particularly useful for reducing or preventing pain associated with arthritis. In some embodiments of the method of preventing or reducing pain, the subject has osteoarthritis. In some embodiments, the subject has unilateral osteoarthritis of the knee. In some embodiments, the subject has at least Grade 2 osteoarthritis of the knee joint on the Kellgren-Lawrence (KL) grading scale of 0 to 4 as per central reader evaluation. In some embodiments, the subject has Grade 2 osteoarthritis of the knee joint on the KL grading scale of 0 to 4 as per central reader evaluation (Kohn et al (2016) Clin Orthop Relat Res 474: 1886-1893 and Altman et al. (1986) Arthritis Rheum.; 29(8):1039-49). The KL classification system is based on radiographic assessment of the knee joint with Grade 0 characterized by no radiographic features of osteoarthritis, thereby signifying no presence of OA; Grade 1 characterized by doubtful narrowing of joint space; Grade 2 characterized by possible joint space narrowing and the presence of osteophytes; Grade 3 characterized by definite joint space narrowing and multiple osteophytes; and Grade 4 characterized by marked joint space narrowing, severe sclerosis and large osteophytes, thereby signifying severe OA

The efficacy of pain control can be measured by asking a patient to rate the quality and intensity of pain experienced according to a number of different scales. A verbal pain scale uses words to describe a range from no pain, mild pain, moderate pain and severe pain with a score from 0-3 assigned to each. Alternatively a patient may be asked to rate their pain according to a numerical pain scale from 0 (no pain) to 10 (worst possible pain). On a visual analog scale (VAS) a vertical or horizontal line has words to describe pain from no pain to worst possible pain and the patient is asked to mark the line at the point that represents their current level of pain. The McGill pain index enables patients to describe both the quality and intensity of pain by selecting words that best describe their pain from a series of short lists e.g. pounding, burning, pinching. Other pain scales can be used for adults who experience difficulty using VAS or numerical scales e.g. FACES or for non-verbal patients e.g. Behavioural rating scale. The functional activity score relates how impeded a patient is by their pain by asking them to carry out a task related to the painful area. Improvements in pain score using these types of scale would potentially indicate an improvement in efficacy of an analgesic.

The baseline level of pain suffered by a subject may be determined before any of the binding molecules disclosed herein are administered to the subject. In some embodiments, the subject has a mean Western Ontario and McMaster Universities Osteoarthritis Index (WOMAC) pain score of at least 5 in a joint as measured using the pain subscale of the WOMAC index at baseline.

The WOMAC multiscale index is used to assess pain, stiffness, and joint functionality in subjects with OA of the knee or hip. The WOMAC pain subscale is a widely-used, patient reported outcome measurement tool to evaluate participants with OA of the knee (Lundgren-Nilsson et al. Patient-reported outcome measures in osteoarthritis: a systematic search and review of their use and psychometric properties. RMD Open. 2018 Dec. 16; 4(2):e000715). consists of 5 questions assessing subject's pain due to OA in the target knee. Each question is scored on an NRS scale from 0 to 10, and the WOMAC pain subscale score is calculated as the mean score from all 5 questions, where higher scores represent higher pain. The WOMAC physical function subscale consists of 17 questions assessing subject's difficulty in performing activities of daily living due to OA in the target knee. Each question is scored on an NRS scale from 0 to 10, and the WOMAC pain subscale score is calculated as the mean score from all 17 questions, where higher scores represent worse function. The WOMAC stiffness function subscale consists of 2 questions assessing stiffness due to OA in the target knee. Stiffness is defined as a sensation of decreased ease of movement in the target knee. Each question is scored on an NRS scale from 0 to 10, and the WOMAC pain subscale score is calculated as the mean score from the 2 questions, where higher scores represent higher stiffness. As used herein, the baseline WOMAC score is defined as the WOMAC score on the day of administration of the binding agent.

In some embodiments, the subject has a mean pain intensity score of at least 5 in a joint as measured on a pain numerical rating (NRS) scale at baseline. The NRS is an 11-point Likert scale used to assess pain, where subjects are asked to describe their average pain in the index knee by identifying a number from 0=“no pain” to 10=“most severe pain imaginable over the previous 24 hours” (see, Alghadir et al. Test-retest reliability, validity, and minimum detectable change of visual analog, numerical rating, and verbal rating scales for measurement of osteoarthritic knee pain. J Pain Res. 2018 Apr. 26; 11:851-6). As used herein, the baseline NRS score is defined as the mean of daily NRS pain scores recorded from Day-7 to Day −1 (inclusive) before initiation of treatment with any of the binding molecules disclosed herein.

Efficacy of pain reduction or prevention may be ascertained by comparing changes in the level of pain in a subject administered any of the binding molecules disclosed herein with changes in the level of pain in a control subject not administered any of the binding molecules disclosed herein. In some embodiments, any of the methods or dosage regimens disclosed herein reduces pain by at least 1, 1.5, 2, 2.5, 3, 3.5, 4, 4.5, 5.5 or 6 points on the Western Ontario and McMaster Universities Osteoarthritis Index (WOMAC) scale (if scaled on a scale of 1-10) as compared to the WOMAC score in a control subject not administered the binding molecule (e.g., a control subject administered a placebo). In some embodiments, any of the methods or dosage regimens disclosed herein reduces pain by at least 1, 1.5, 2, 2.5, 3, 3.5, or 4 points on the WOMAC scale (if scaled on a scale of 0-4) as compared to the WOMAC score in a control subject not administered the binding molecule (e.g., a control subject administered a placebo).

In some embodiments, any of the methods or dosage regimens disclosed herein method reduces pain by at least 1, 1.5, 2, 2.5, 3, 3.5, 4, 4.5, 5, 5.5 or 6 points on the pain numerical rating scale (NRS) (if scaled on a scale of 1-10) as compared to the NRS score in a control subject not administered the binding molecule. In some embodiments, the pain reduction is observed following a single dose administration of the binding molecule to the subject.

Efficacy of pain reduction or prevention may be ascertained by comparing changes in the level of pain in a subject administered any of the binding molecules disclosed herein with the level of pain in the subject at baseline. In some embodiments, any of the methods or dosage regimens disclosed herein reduces the subject's WOMAC pain subscale from baseline. In some embodiments, any of the binding molecules disclosed herein are administered in a fixed dose every 2 weeks for 12 weeks and the method reduces the subject's WOMAC pain subscale score from baseline by at least 12 weeks after first administration with any of the binding molecules disclosed herein. In some embodiments, any of the methods or dosage regimens disclosed herein reduces the subject's WOMAC pain subscale score from baseline by at least 20%, at least 30%, at least 40%, or at least 50%. In some embodiments, any of the methods or dosage regimens disclosed herein reduces the subject's WOMAC pain subscale score from baseline by at least 30%. In some embodiments, any of the methods or dosage regimens disclosed herein reduces the subject's WOMAC pain subscale score from baseline by at least 50%.

In some embodiments, any of the methods or dosage regimens disclosed herein reduces the subject's WOMAC physical subscale score from baseline. In some embodiments, any of the binding molecules disclosed herein are administered in a fixed dose every 2 weeks for 12 weeks and the method reduces the subject's WOMAC physical subscale score from baseline by at least 12 weeks after first administration with any of the binding molecules disclosed herein. In some embodiments, any of the methods or dosage regimens disclosed herein reduces the subject's WOMAC physical subscale score from baseline by at least 20%, at least 30%, at least 40%, or at least 50%. In some embodiments, any of the methods or dosage regimens disclosed herein reduces the subject's WOMAC physical subscale score from baseline by at least 30%. In some embodiments, any of the methods or dosage regimens disclosed herein reduces the subject's WOMAC physical subscale score from baseline by at least 50%.

In some embodiments, any of the methods or dosage regimens disclosed herein reduces the subject's weekly average of daily NRS pain score from baseline. In some embodiments, any of the binding molecules disclosed herein are administered in a fixed dose every 2 weeks for 12 weeks and the method reduces the subject's weekly average of daily NRS pain score from baseline by at least 12 weeks. In some embodiments, any of the methods or dosage regimens disclosed herein reduces the subject's weekly average of daily NRS pain score from baseline by at least 20%, at least 30%, at least 40%, or at least 50%. In some embodiments, any of the methods or dosage regimens disclosed herein reduces the subject's weekly average of daily NRS pain score from baseline by at least 30%. In some embodiments, any of the methods or dosage regimens disclosed herein reduces the subject's weekly average of daily NRS pain score from baseline by at least 50%.

In some embodiments, any of the methods or dosage regimens disclosed herein improves the Patient Global Assessment (PGA) of osteoarthritis from baseline. As used herein, the baseline PGA is defined as the PGA score on the day of administration of the binding agent. The PGA is a 5-point Likert scale used to assess symptoms and activity impairment due to OA of the knee (see, e.g., Nikiphorou et al (2016) Arthritis Res Ther 18:251). Subjects are asked to identify a number from 1=very good (asymptomatic and no limitation of normal activities) to 5=very poor (very severe symptoms which are intolerable and inability to carry out all normal activities) based on the question “Considering all the ways that OA of the knee affects you, how are you feeling today?” In some embodiments, any of the binding molecules disclosed herein are administered in a fixed dose every 2 weeks for 12 weeks and the method improves the PGA of osteoarthritis from baseline by at least 12 weeks. In some embodiments, any of the methods or dosage regimens disclosed herein improves the PGA of osteoarthritis by at least 2 points.

Efficacy of pain reduction or prevention may be ascertained by measuring changes in the levels of biomarkers in a subject. In some embodiments, the method of preventing or reducing pain suppresses NGF activity in the subject by at least 30%, 40%, 50%, 60%, 70%, 80%, 90% or 100% as compared to the NGF activity in a control subject not administered the binding molecule (e.g., a control subject administered a placebo). In some embodiments, the method suppresses NGF activity in the subject by at least 40% as compared to the NGF activity in a control subject not administered the binding molecule. In some embodiments, the NGF suppression is observed following a single dose administration of the binding molecule to the subject. In some embodiments, the NGF suppression is observed following administration of multiple doses of the binding molecule to the subject.

In some embodiments, the method of preventing or reducing pain suppresses CXCL-13 levels in the subject by at least 5%, 10%, 20%, 30%, 40%, 50%, 60%, 70%, 80%, 90% or 100% as compared to the CXCL-13 levels in a control subject not administered the binding molecule (e.g., a control subject administered a placebo). In some embodiments, the CXCL-13 suppression is observed following a single dose administration of the binding molecule to the subject. In some embodiments, the CXCL-13 suppression is observed following administration of multiple doses of the binding molecule to the subject.

In certain aspects, formulations are prepared for storage and use by combining a TNFα and NGF antagonist multifunctional polypeptide, e.g., a multispecific binding molecule as provided herein, with a pharmaceutically acceptable vehicle (e.g., carrier, excipient) (Remington, The Science and Practice of Pharmacy 20th Edition Mack Publishing, 2000). Suitable pharmaceutically acceptable vehicles include, but are not limited to, nontoxic buffers such as phosphate, citrate, and other organic acids; salts such as sodium chloride; antioxidants including ascorbic acid and methionine; preservatives (e.g. octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosacchandes, disaccharides, glucose, mannose, or dextrins; chelating agents such as EDTA; sugars such as sucrose, mannitol, trehalose or sorbitol; salt-forming counter-ions such as sodium; metal complexes (e.g., Zn-protein complexes); and non-ionic surfactants such as TWEEN or polyethylene glycol (PEG).

Multifunctional polypeptides of the present disclosure may be formulated in liquid, semi-solid or solid forms depending on the physicochemical properties of the molecule and the route of delivery. Formulations may include excipients, or combinations of excipients, for example: sugars, amino acids and surfactants. Liquid formulations may include a wide range of polypeptide concentrations and pH. Solid formulations may be produced by lyophilisation, spray drying, or drying by supercritical fluid technology, for example. In some embodiments, any of the formulations described herein is a lyophilized formulation.

A pharmaceutical composition provided herein can be administered in any number of ways for either local or systemic treatment. Administration can be topical (such as to mucous membranes including vaginal and rectal delivery) such as transdermal patches, ointments, lotions, creams, gels, drops, suppositories, sprays, liquids and powders; pulmonary (e.g., by inhalation or insufflation of powders or aerosols, including by nebulizer; intratracheal, intranasal, epidermal and transdermal); oral; or parenteral including intravenous, intraarterial, subcutaneous, intraperitoneal or intramuscular injection or infusion; or intracranial (e.g., intrathecal or intraventricular) administration.

A TNFα and NGF antagonist multifunctional polypeptide as provided herein can be further combined in a pharmaceutical combination formulation, or dosing regimen as combination therapy, with a second (or third) compound having anti-nociceptive properties.

For the treatment of pain, the appropriate dosage of a TNFα and NGF antagonist multifunctional polypeptide, e.g., a multispecific binding molecule as provided herein depends on the type of pain to be treated, the severity and course of the pain, the responsiveness of the pain, whether the multifunctional polypeptide is administered for therapeutic or prophylactic purposes, previous therapy, patient's clinical history, and so on all at the discretion of the treating physician. The multifunctional polypeptide can be administered one time or over a series of treatments lasting from several days to several months to maintain effective pain control. Optimal dosing schedules can be calculated from measurements of drug accumulation in the body of the patient and will vary depending on the relative potency of an individual antibody or polypeptide. The administering physician can easily determine optimum dosages, dosing methodologies and repetition rates.

Administration of a multifunctional polypeptide, e.g., a multispecific binding molecule as provided herein can provide “synergy” and prove “synergistic,” i.e. the effect achieved when the active ingredients used together is greater than the sum of the effects that results from using the compounds separately. A synergistic effect can be attained when the active ingredients are administered as a single, multifunctional fusion polypeptide.

### Pain

In its broadest usage, “pain” refers to an experiential phenomenon that is highly subjective to the individual experiencing it, and is influenced by the individual's mental state, including environment and cultural background. “Physical” pain can usually be linked to a stimulus perceivable to a third party that is causative of actual or potential tissue damage. In this sense, pain can be regarded as a “sensory and emotional experience associated with actual or potential tissue damage, or described in terms of such damage,” according to the International Association for the Study of Pain (IASP). However, some instances of pain have no perceivable cause. For example, psychogenic pain, including exacerbation of a pre-existing physical pain by psychogenic factors or syndromes of a sometimes persistent, perceived pain in persons with psychological disorders without any evidence of a perceivable cause of pain. “Pain” in the context of the present invention may be, or may include, any of the types of pain disclosed herein.

### Types of Pain

In the context of the present invention, pain includes nociceptive pain, neuropathic/neurogenic pain, breakthrough pain, allodynia, hyperalgesia, hyperesthesia, dysesthesia, paresthesia, hyperpathia, phantom limb pain, psychogenic pain, anesthesia dolorosa, neuralgia, neuritis. Other categorizations include malignant pain, anginal pain, and/or idiopathic pain, complex regional pain syndrome I, complex regional pain syndrome II. Types and symptoms of pain need not be mutually exclusive. These terms are intended as defined by the IASP.

Nociceptive pain is initiated by specialized sensory nociceptors in the peripheral nerves in response to noxious stimuli, encoding noxious stimuli into action potentials. Nociceptors, generally on Aδ fibers and (Polymodal) C fibers, are free nerve endings that terminate just below the skin, in tendons, joints, and in body organs. The dorsal root ganglion (DRG) neurons provide a site of communication between the periphery and the spinal cord. The signal is processed through the spinal cord to the brainstem and thalamic sites and finally to the cerebral cortex, where it usually (but not always) elicits a sensation of pain. Nociceptive pain can result from a wide variety of a chemical, thermal, biological (e.g., inflammatory) or mechanical events that have the potential to irritate or damage body tissue, which are generally above a certain minimal threshold of intensity required to cause nociceptive activity in nociceptors.

Neuropathic pain is generally the result of abnormal functioning in the peripheral or central nervous system, giving rise to peripheral or central neuropathic pain, respectively. Neuropathic pain is defined by the IASP as pain initiated or caused by a primary lesion or dysfunction in the nervous system. Neuropathic pain often involves actual damage to the nervous system, especially in chronic cases. Inflammatory nociceptive pain is generally a result of tissue damage and the resulting inflammatory process. Neuropathic pain can persist well after (e.g., months or years) beyond the apparent healing of any observable damage to tissues.

In cases of neuropathic pain, sensory processing from an affected region can become abnormal and innocuous stimuli (e.g., thermal, touch/pressure) that would normally not cause pain may do so (i.e., allodynia) or noxious stimuli may elicit exaggerated perceptions of pain (i.e., hyperalgesia) in response to a normally painful stimulus. In addition, sensations similar to electric tingling or shocks or “pins and needles” (i.e., paresthesias) and/or sensations having unpleasant qualities (i.e., dysesthesias) may be elicited by normal stimuli. Breakthrough pain is an aggravation of pre-existing chronic pain. Hyperpathia is a painful syndrome resulting from an abnormally painful reaction to a stimulus. The stimulus in most of the cases is repetitive with an increased pain threshold, which can be regarded as the least experience of pain that a patient can recognize as pain.

Examples of neuropathic pain include tactile allodynia (e.g., induced after nerve injury) neuralgia (e.g., post herpetic (or post-shingles) neuralgia, trigeminal neuralgia), reflex sympathetic dystrophy/causalgia (nerve trauma), components of cancer pain (e.g., pain due to the cancer itself or associated conditions such as inflammation, or due to treatment such as chemotherapy, surgery or radiotherapy), phantom limb pain, entrapment neuropathy (e.g., carpal tunnel syndrome), and neuropathies such as peripheral neuropathy (e.g., due to diabetes, HIV, chronic alcohol use, exposure to other toxins (including many chemotherapies), vitamin deficiencies, and a large variety of other medical conditions). Neuropathic pain includes pain induced by expression of pathological operation of the nervous system following nerve injury due to various causes, for example, surgical operation, wound, shingles, diabetic neuropathy, amputation of legs or arms, cancer, and the like. Medical conditions associated with neuropathic pain include traumatic nerve injury, stroke, multiple sclerosis, syringomyelia, spinal cord injury, and cancer.

A pain-causing stimulus often evokes an inflammatory response which itself can contribute to an experience of pain. In some conditions pain appears to be caused by a complex mixture of nociceptive and neuropathic factors. For example, chronic pain often comprises inflammatory nociceptive pain or neuropathic pain, or a mixture of both. An initial nervous system dysfunction or injury may trigger the neural release of inflammatory mediators and subsequent neuropathic inflammation. For example, migraine headaches can represent a mixture of neuropathic and nociceptive pain. Also, myofascial pain is probably secondary to nociceptive input from the muscles, but the abnormal muscle activity may be the result of neuropathic conditions.

According to the method of controlling pain (e.g. reducing or preventing pain) provided herein, the administration of any of the binding molecules disclosed herein is sufficient to control pain (e.g. reduce or prevent pain) in the subject in need of pain control. In some embodiments, pain reduction is observed following a single dose administration of any of the binding molecules disclosed herein to the subject. In some embodiments, pain reduction is observed following administration of multiple doses of any of the binding molecules disclosed herein to the subject. In particular embodiments, the disclosure provides for methods or dosage regimens for reducing or preventing pain associated with osteoarthritis. In some embodiments, the pain associated with osteoarthritis is knee pain associated with osteoarthritis.

In some embodiments of the method of preventing or reducing pain, the pain is acute pain, short-term pain, persistent or chronic nociceptive pain, or persistent or chronic neuropathic pain. In some embodiments, the pain comprises chronic pain. In some embodiments the pain is associated with joint inflammation, such as inflammation of the knee or hip. In some embodiments, the pain comprises osteoarthritic pain. In some embodiments, the pain comprises osteoarthritic pain of the knee.

### Kits Comprising TNFα and NGF Antagonists

This disclosure provides kits that comprise a TNFα and NGF antagonist multifunctional polypeptide, e.g., a multispecific binding molecule, as provided herein, that can be used to perform the methods described herein. In certain aspects, a kit comprises at least multifunctional fusion polypeptide comprising a TNFα antagonist and an NGF antagonist, e.g., a polypeptide comprising an amino acid sequence of SEQ ID NO: 14 or 17, in one or more containers. One skilled in the art will readily recognize that the disclosed TNFα and NGF antagonists provided herein can be readily incorporated into one of the established kit formats, which are well known in the art.

### EXAMPLES

The disclosure now being generally described, it will be more readily understood by reference to the following examples, which are included merely for purposes of illustration of certain aspects and embodiments of the present disclosure, and are not intended to limit the disclosure.

### Example 1—Construction and Characterization of an Anti NGF scFv/TNFR2-Fc Multispecific Binding Molecule

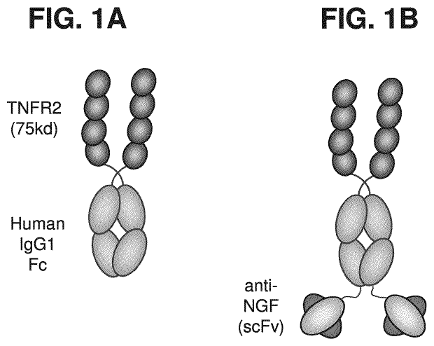

A multifunctional molecule, specifically, a multispecific binding molecule comprising an anti NGF antibody domain and a TNFR2-Fc domain was produced as follows. The anti-NGF antibody scFv fragment was fused to the C-terminus of a TNFR2-Fc fusion protein (SEQ ID NO: 13) via the heavy chain CH3 domain, according to the Bs3Ab format described in Dimasi, N., et al., J Mol Biol. 393:672-92 (2009), and in PCT Publication No. WO 2013/070565. A diagram of the structure is shown in FIG. 1. DNA constructs encoding the TNFR2-Fc polypeptide and the multispecific binding molecule were synthesized by GeneArt (Invitrogen). For the multispecific binding molecule, an anti-NGF scFv comprising the VH (SEQ ID NO: 3) and VL (SEQ ID NO: 7) domains of MEDI-578 joined together via a 15 amino acid linker sequence (GGGGS)₃ (SEQ ID NO: 15) was constructed. The N-terminus of the scFv was fused, via a 10-amino-acid linker sequence (GGGGS)₂, to the C-terminus of SEQ ID NO: 13. This multispecific binding molecule is referred to herein as TNFR2-Fc_VH #4. The DNA construct encoding the multispecific binding molecule was engineered to contain a stop codon and an EcoRI restriction site at the 3′ end for cloning into the Bs3Ab expression vector. The DNA sequence encoding TNFR2-Fc_VH #4 is presented as SEQ ID NO: 16 and its amino acid sequence as SEQ ID NO: 14.

The thermostability of the TNF-NGF multispecific binding molecule was improved by the addition of an inter-chain disulphide bond between the VH and VL domains of the MEDI-578 scFv portion of the multispecific binding molecule. This was done by introducing a G→C mutation at amino acid 44 of the VH domain (SEQ ID NO: 94) and at amino acid 103 of the VL domain (SEQ ID NO: 95). This clone was designated TNFR2-Fc_varB. The amino acid sequence of TNFR2-Fc_varB is presented as SEQ ID NO: 17. A DNA sequence encoding TNFR2-Fc_varB is presented as SEQ ID NO: 18. A codon optimized DNA sequence encoding TNFR2-Fc_varB is presented in SEQ ID NO: 99. TNFR2-Fc_varB further differs from TNFR2-Fc_VH #4 in that the 15 amino acid linker sequence (GGGGS)₃ joining the VH and VL of the scFv portion is replaced with a 20 amino acid linker (GGGGS)₄ (SEQ ID NO: 19). Differential scanning fluorimetry (DSF) was used to measure the Tm of TNFR2-Fc_VH #4 and TNFR2-Fc_varB. This method measures the incorporation of a fluorescent dye, Sypro Orange (Invitrogen), which binds to hydrophobic surfaces revealed during protein domain unfolding upon exposure to elevated temperatures. In the DSF assay, the Tm of TNFR2-Fc_VH #4 was 62° C., whereas the Tm of TNFR2-Fc_varB was 66° C. Therefore, the addition of the inter-chain disulphide bond in the MEDI-578 scFv portion of the multispecific molecule improved the thermostability of the molecule by 4° C.

The TNFR2-Fc protein and TNFR2-Fc_VH #4 were transiently expressed in suspension CHO cells using Polyethylenimine (PEI) (Polysciences) as the transfection reagent. The cells were maintained in CD-CHO medium (Life Technologies). Culture harvests from small-scale transfections were purified using 1 ml HiTrap MabSelect SuRe™ affinity chromatography in accordance with the manufacturer's protocol (GE Healthcare) and were subsequently buffer exchanged in 1% sucrose, 100 mM NaCl, 25 mM L-arginine hydrochloride, and 25 mM sodium phosphate (pH 6.3). The purity of the recombinant proteins was analyzed using SDS-PAGE under reducing conditions and using analytical size-exclusion chromatography (see method below), and concentrations were determined by reading the absorbance at 280 nm using theoretically determined extinction coefficients.

Small scale transient expression and protein A column purification of the TNFR2-Fc fusion protein and the TNF-NGF multispecific construct, TNFR2-Fc_VH #4, produced yields of 36.6 and 79.9 mg L⁻¹ respectively.

A larger batch of TNFR2-Fc_VH #4 was produced as follows. A crude culture harvest from a large-scale transfection (up to 6 L) was filtered using depth filtration and loaded onto a 1.6×20 cm Protein A agarose column (GE Healthcare) pre-equilibrated with buffer A (phosphate buffered saline pH 7.2). The column was then washed with buffer A and the product eluted in a step gradient of buffer B (50 mM Sodium Acetate pH<4.0). The product was further purified by loading onto a 1.6×20 cm Poros HS 50 column (Applied Biosystems) pre-equilibrated in buffer C (50 mM Sodium Acetate buffer pH<5.5), washed in buffer C and then subsequently the product was eluted in a linear gradient from 0 to 1 M NaCl in 50 mM Sodium Acetate buffer pH<5.5. The resulting eluates were analysed by Size Exclusion HPLC. The protein concentration was determined by A280 spectroscopy with a Beckman DU520 spectrophotometer using a calculated extinction coefficient of 1.36.

### Methods for Characterization of TNFR2-Fc_VH #4

Western blot analysis was carried out using standard protocols. Proteins were transferred onto the polyvinylidene fluoride membrane (Life Technologies) using the Xcell SureLock™ system (Invitrogen) according to the manufacturer's instructions. The membrane was blocked with 3% (w/v) skim milk powder in phosphate-buffered saline (PBS) for 1 h at room temperature. Western blots were developed using standard protocols with HRP-conjugated anti-human IgG Fc-specific antibody (Sigma).

Size exclusion HPLC was performed using a Gilson HPLC system (Isocratic pump-307, UV/Vis-151 detector, Liquid Handler-215 and Injection Module-819) with a Phenomenex BioSep-SEC-53000 (300×7.8 mm) column with a mobile phase of D-PBS (life Technologies) at a flow rate of 1 ml/min. Twenty-five μL samples were injected onto the column and separation of protein species was monitored at A280 nm

Enzymatic deglycosylation of small-scale purified TNFR2-Fc_VH #4 was performed using an EDGLY kit (Sigma Aldrich) according to the manufacturer's protocols. Proteins were deglycosylated under both denatured and native conditions. For denatured proteins, 30 lag of protein was deglycosylated with PNGase F, β-glycosidase, and α-(2→3, 6, 8, 9)-neuraminidase, 13-N-acetylglucosaminidase and β-(1→4)-galactosidase for 3 h at 37° C. Under native conditions, 35 μg of protein was deglycosylated with the same set of enzymes as above for 3 days at 37° C. The deglycosylated proteins were analyzed by coomassie stained SDS-PAGE and by western blot using standard assay protocols.

N-terminal amino acid sequencing of TNFR2-Fc_VH #4 was carried out as follows. Approximately 2 μg of TNFR2-Fc_VH #4 was run on an SDS-PAGE gel using standard protocols. Proteins were transferred onto the PVDF membrane using the Xcell SureLock™ system (Invitrogen) according to the manufacturer's instructions. The membrane was stained with 0.1% (w/v) amidoblack for approximately 15 min on an orbital shaking platform then washed with dH₂O to reduce background staining of the PVDF membrane. The membrane was air-dried prior to N-terminal sequencing. The bands of interest were cut out and sequence determination of the N-terminus of the multispecific binding molecule was performed on an Applied Biosystems 494 HT sequencer (Applied Biosystems, San Francisco, CA, U.S.A.) with on-line phenylthiohydantoin analysis using an Applied Biosystems 140A micro HPLC.

### Characterization Results

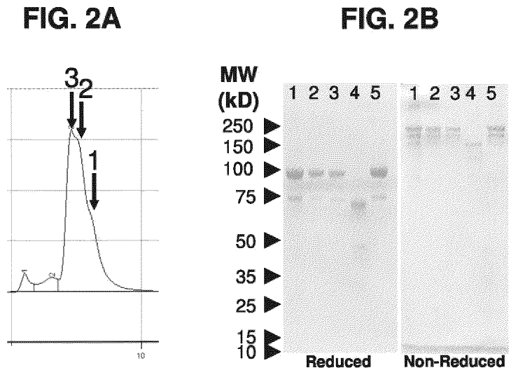

Purified TNFR2-Fc_VH #4 and TNFR2-Fc proteins were profiled by SEC-HPLC for levels of aggregate, monomer and protein fragmentation (FIGS. 2A and 2B). The main peak comprising monomer constituted approximately 90% of the total protein present with the remaining approximately 10% of the protein mass with a lower column retention time indicating the presence of higher order species or aggregates. However, the monomer peak from the SEC-HPLC had two pronounced shoulders indicating that the protein within this peak was not a single species. SDS-PAGE analysis with coomassie staining showed two distinct bands for TNFR2-Fc_VH #4 (at approx. 100 and 75 kD) and similarly two distinct bands for the TNFR2-Fc fusion protein also (at approx. 70 and 45 kD) under reducing conditions (FIG. 2B). Under non-reducing conditions, three major bands were present for TNFR2-Fc_VH #4 (between 150 and 250 kD) and one major band and one minor band for the TNFR2-Fc fusion protein at approx. 150 and 120 kD respectively. Since the molecular mass difference between the two bands under reducing conditions was approximately equivalent to the size of a scFv fragment (˜26.5 kD) further analysis was performed in order to understand in what forms the multispecific binding molecule were being generated. Mass spectroscopic analysis under native conditions confirmed the SDS-PAGE data, that for two separate purified protein preparations there were three molecular masses present in the purified TNFR2-Fc_VH #4 preparation at approximately 125, 152 and 176 kD (FIG. 2C).

If the banding pattern observed by SDS-PAGE gel was due to differential glycosylation of TNFR2-Fc_VH #4, then upon deglycosylation this would be resolved back down to a single band. However, the banding pattern was maintained under both reducing and non-reducing conditions when TNFR2-Fc_VH #4 was deglycosylated either as a native protein or as denatured protein (data not shown). Western blot staining of both the glycosylated and deglycosylated TNFR2-Fc_VH #4 with a polyclonal anti-human IgG Fc specific antibody showed that both the full length expected band and the lower molecular mass band were reactive with anti-Fc specific antibodies (data not shown).

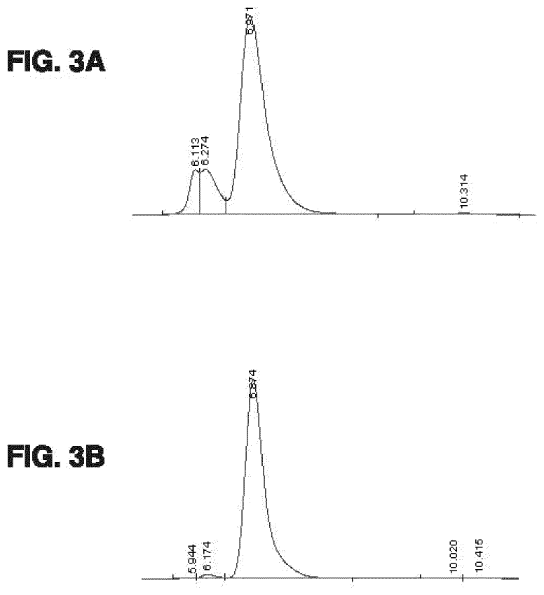

Final identification of the truncated product was made by N-terminal amino acid sequencing of the protein. This revealed that the first 8 amino acids of the N-terminus of the truncated protein to be SMAPGAVH corresponding to amino acids 176 to 183 of the TNFR2-Fc_VH #4 sequence (SEQ ID NO: 14). This represented a 175 amino acid truncation at the N-terminus of TNFR2-Fc_VH #4, which left only 42 amino acids of the TNFR2 domain. This allows us to accurately interpret the mass data from the SDS-PAGE, mass spectroscopy and SEC-HPLC analysis. There were three possible combinations of TNFR2-Fc_VH #4 dimers and all were present in the purified protein preparations: (1) full length homodimer, (2) a heterodimer of full length and truncated species, and (3) a homodimer of truncated species. In order to accurately measure biological activity both in vitro and in vivo, a preparation of the full-length homodimer was generated by a two-step column chromatography process. In the first step, post Protein A purification, the product contained 80.5% monomer (FIG. 3A) and after the second column purification step (SP sepharose) the monomer percentage was 97.8% (FIG. 3B). The yield over the whole process was 7.3%.

### Example 2—Thermal Stability Analysis by Differential Scanning Calorimetry (DSC)

An automated MicroCal VP-Capillary DSC (GE Healthcare, USA) was used for the calorimetric measurements. Protein samples were tested at 1 mg/mL in 25 mM Histidine/Histidine-HCl buffer pH 6.0. The protein samples and buffer were subjected to a linear heat ramp from 25° C. to 100° C. at a rate of 95° C. per hour. The buffer was subtracted as a reference from the protein sample using Origin 7 software and the thermal transitions were determined.

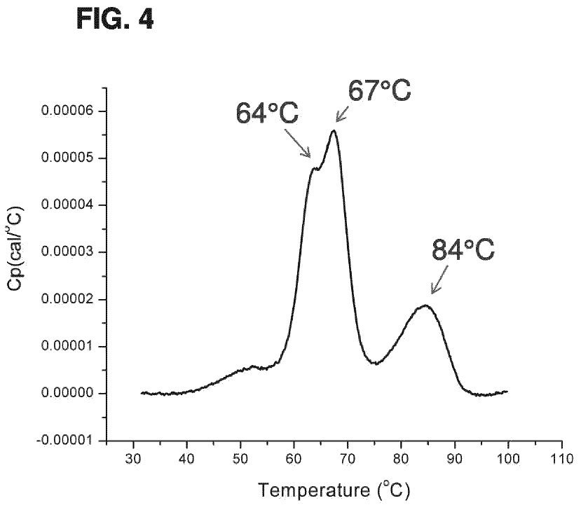

The thermogram for TNFR2-Fc_VH #4 (FIG. 4) shows three distinct unfolding transitions with denaturation temperatures (Tm) of 64, 67, and 84° C. We deduced that the Tm of 64° C. corresponded with the denaturation of both the TNFR2 domain and the anti-NGF scFv domain, with the Tms of 67° C. and 84° C. being typical of the denaturation Tms for IgG1 CH2 and CH3 domains respectively (e.g. Dimasi, N., et al., J Mol Biol. 393:672-92 (2009), and PCT Publication No. WO 2013/070565). While not wishing to be bound by theory, scFv generally have lower denaturation temperatures than the other antibody domains, and their unfolding is characterized by a single transition event (Roberge et al., 2006, Jung et al., 1999, Tischenko et al., 1998).

### Example 3—Confirmation of Antigen Binding to TNFR2-Fc_VH #4

### A. Single and Dual Antigen Binding by ELISA

Nunc Maxisorp wells were coated at 4° C. overnight with 50 μl of TNFα (R&D Systems) diluted to 5 μg ml⁻¹ in PBS (pH 7.4). The following day the coating solution was removed and the wells blocked with 150 μl of blocking buffer [3% skimmed milk-PBS]for 1 h at room temperature. The wells were rinsed three times in PBS, prior to the addition of 50 μl of a dilution series of TNFR2-Fc_VH #4 made in blocking buffer. After 1 h at room temperature, the wells were washed three times in PBS-Tween 20 (0.1% v/v; PBS-T). Fifty microliters of biotinylated NGF was then added to the wells and incubated for a further hour at room temperature, prior to washing as above and addition of 50 μl of streptavidin-HRP (1:100). After 1 hour at room temperature, the wells were washed with PBS-T, 50 μl of 3,3′,5,5′-tetramethylbenzidine substrate added and the color allowed to develop. The reaction was stopped by the addition of 1M H₂SO₄ and the absorbance at 450 nm was measured using a microtiter plate reader. The resulting data were analyzed using Prism 5 software (GraphPad, San Diego, CA). For the single antigen binding ELISA, the wells were coated with either TNFα or NGF-biotin as above and antibody binding detected with anti-Human IgG Fc specific HRP conjugated antibody (1:5000), and color developed as above.

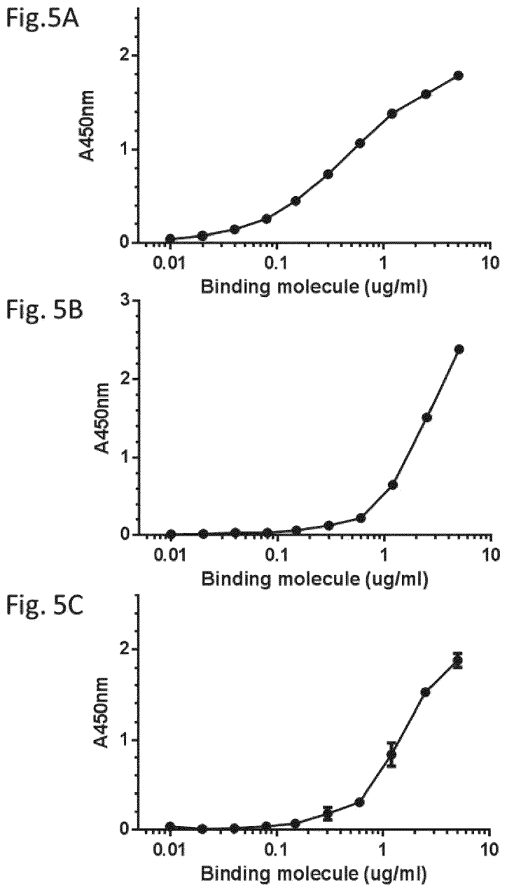

The ELISA results are shown in FIG. 5. TNFR2-Fc_VH #4 was designed to bind to both TNFα and NGF antigens. Single antigen binding was performed by first immobilizing one antigen onto a 96-well microtiter plate, followed by the addition of serial dilutions of TNFR2-Fc_VH #4. Specific binding was detected by using a horseradish peroxidase (HRP)-conjugated anti-IgG Fc specific antibody. For the dual antigen binding ELISA, the first antigen, TNFα was immobilized on the ELISA plate, and then a serial dilution of TNFR2-Fc_VH #4 was added, followed by the addition of the second biotinylated antigen, NGF at a fixed concentration. Specific binding was then detected using an HRP-conjugated streptavidin. TNFR2-Fc_VH #4 bound to TNFα and NGF in the single antigen binding ELISA (FIGS. 5A and B). In the dual antigen binding ELISA, TNFR2-Fc_VH #4 bound to both TNFα and NGF simultaneously (FIG. 5C).

### B. Simultaneous Antigen Binding by Surface Plasmon Resonance

Simultaneous antigen binding experiments were carried out essentially as described in Dimasi, N., et al., J Mol Biol. 393:672-92 (2009) using a BIAcore 2000 instrument (GE Healthcare). Briefly, a CMS sensor chip was used to immobilize approximately 1500 resonance units of TNFR2-Fc_VH #4 at 100 nM. The sensor chip surfaces were then used for concurrent binding for TNFα and NGF. The antigens were prepared in HBS-EP buffer [10 mM HEPES (pH 7.4), 150 mM NaCl, 3 mM ethylenediaminetetraacetic acid (EDTA), 0.005% P201. A flow rate of 30 μl/min was used for all binding measurements. For determining the simultaneous binding of the multispecific antibody to TNFα and NGF, 1 μM of TNFα (molecular mass, 17.5 kD) was injected over the sensor chip surface, and upon completion of injection, a mixture of TNFα and NGF (molecular mass, 13.5 kD), both at 1 μM, was then injected. TNFα was included in the mixture with NGF to prevent the signal loss due to TNFα dissociation during NGF binding phase. As a control, a similar binding procedure was performed, and at the last injection only TNFα was added, no further increase in resonance units for this injection indicated that the TNFα was bound at saturating levels. Similar binding and control experiments were performed in which the injection order of TNFα and NGF was reversed.

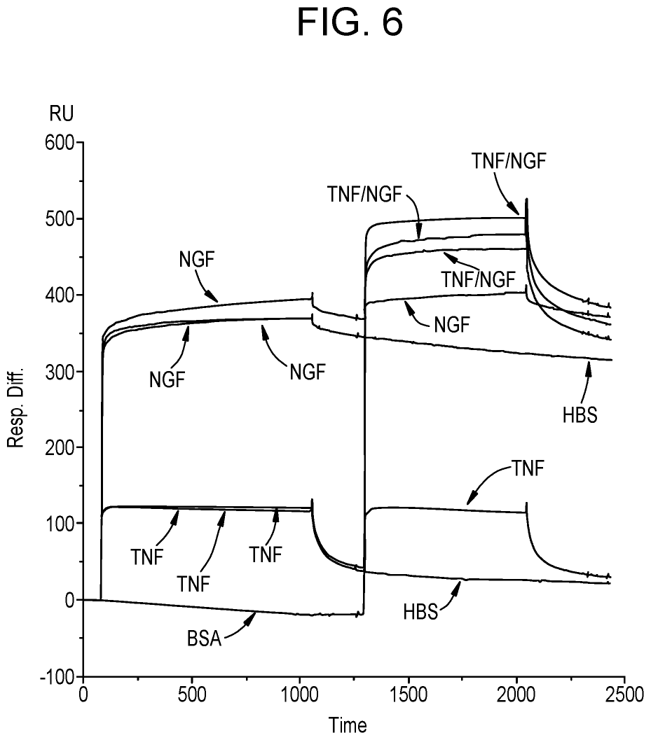

Simultaneous antigen binding of TNFR2-Fc_VH #4 was characterized by surface plasmon resonance. The binding events were analyzed qualitatively in a sequential manner TNFR2-Fc_VH #4 was covalently immobilized on to the sensor chip surface using amine coupling chemistry. Subsequently, the first antigen was injected to give saturating levels of binding to TNFR2-Fc_VH #4, then the second antigen was injected as an equimolar admixture with antigen 1. The binding sensorgram clearly showed that TNFR2-Fc_VH #4 bound simultaneously to TNFα and NGF (FIG. 6). Simultaneous binding of the two antigens occurred regardless of the order of antigen injection.

### Example 4— Inhibition of TF-1 Cell Proliferation Induced by NGF

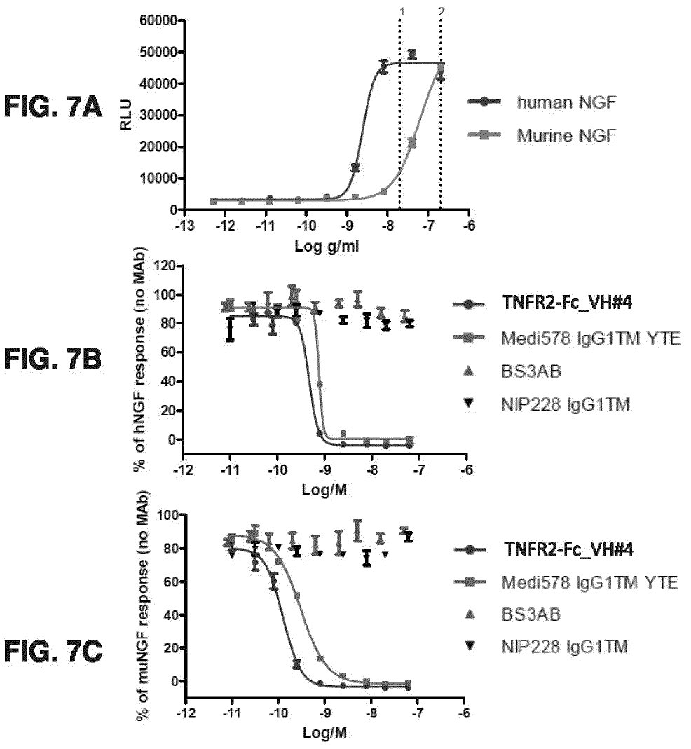

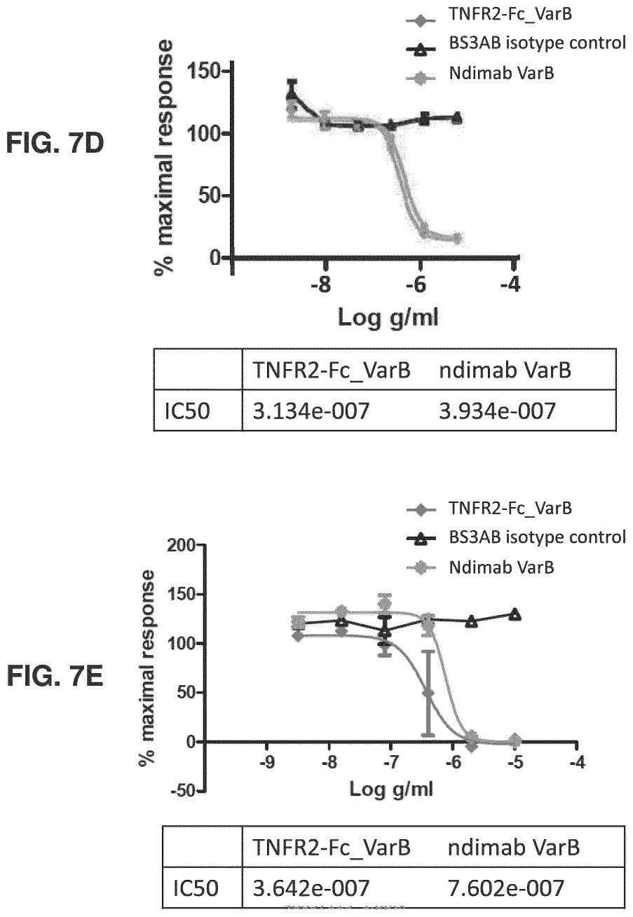

TF-1 cells (ECACC Catalog No. 93022307) were seeded at 1.5×10⁴ cells/well in 50 μl serum free culture media in 96 well tissue culture plate (Corning Costar) and incubated for 18 h at 37° C. with 5% CO₂. Recombinant human (Sigma) or mouse NGF (R&D Systems) were pre-incubated with dilutions of TNFR2-Fc_VH #4, MEDI-578 IgG1 TM YTE, a non-binding IgG1 TM YTE isotype control for MEDI-578, or a non-binding bispecific isotype control R347 Bs3Ab for 30 min at 37° C. in 96 well round bottomed plate (Greiner). Fifty microliters of each sample was then added to cell plate and incubated for 48 h at 37° C. Following the incubation period, 100 μl of cell TITRE GLO® assay buffer (Promega) was added and the plate was incubated for 10 min at 37° C. with 5% CO₂. Luminescence was then measured using standard luminescence protocol. Standard NGF-induced TF-1 proliferation in the absence of antibody is shown in FIG. 7A.

The functional activity of TNFR2-Fc_VH #4 was determined using NGF induced TF-1 proliferation. TNFR2-Fc_VH #4 was able to completely inhibit both human and murine NGF induced proliferation (FIGS. 7B and 7C, respectively). FIG. 7B: TF-1 cells were stimulated with recombinant human NGF corresponding to EC80 concentration. Cells were incubated with ligand with a dilution series of antibody for 48 hrs, after which cell proliferation was quantified by culture for 10 mins with cell TITRE GLO® assay buffer (Promega). FIG. 7C: TF-1 cells were stimulated with recombinant murine NGF corresponding to EC₈₀ concentration. Cells were incubated with ligand with a dilution series of antibody for 48 hrs., after which cell proliferation was quantified by culture for mins with cell TITRE GLO® assay buffer (Promega). These data demonstrate that the NGF inhibitory portion of TNFR2-Fc_VH #4 is biologically active and inhibits NGF induced proliferation with a similar potency to MEDI-578 as an IgG1TM. Similar data was also observed for TNFR2-Fc_varB and another TNF-NGF multispecific binding molecule ndimab var B (FIGS. 7D & 7E). ndimab varB comprises a complete anti-TNFα antibody, i.e., an antibody comprising two complete heavy chains and two complete light chains in an H₂L₂ format, with MEDI-578 scFv fused to the C-terminus of the heavy chain of the anti-TNFα antibody. The light chain of ndimab varB is depicted in SEQ ID NO: 20 and the heavy chain of ndimab varB is depicted in SEQ ID NO: 22.

### Example 5—Inhibition of U937 Cell Apoptosis Induced by TNFα

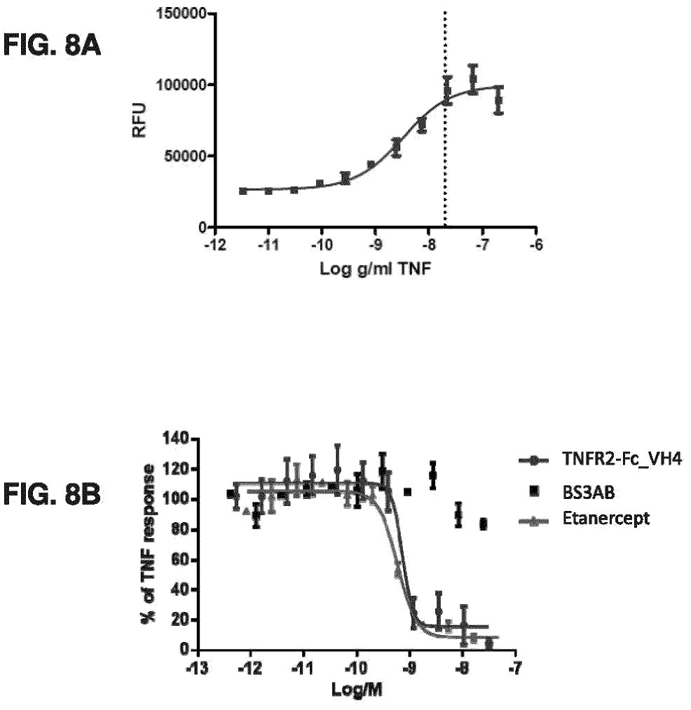

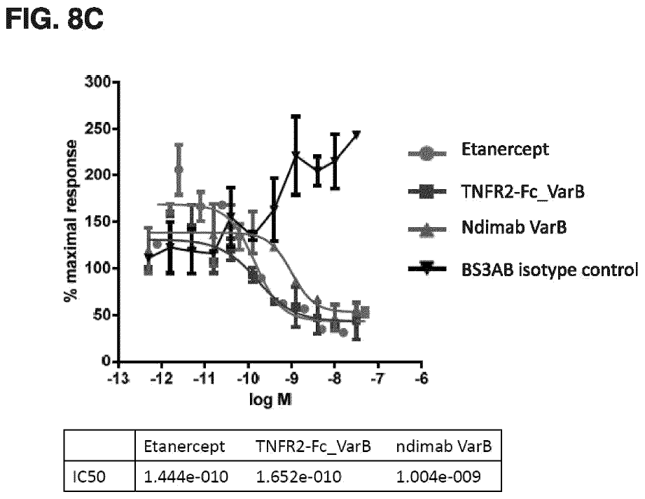

U937 cells (ECACC Cat. No. 85011440) were plated in a black walled 96 well tissue culture plate (Corning Costar) at a concentration of 8×10⁵ cells/well in 50 μl culture media. U937 cells were stimulated with recombinant human TNFα corresponding to EC₈₀ concentration. Cells were incubated with ligand with a dilution series of antibody for 2 hrs, after which caspase 3 activity was quantified by culture for 2 hours with Caspase 3 assay reaction buffer. TNFR2-Fc_VH #4, a non-binding bispecific isotype control, R347 Bs3Ab, and etanercept were pre-incubated with the cells for 30 min at 37° C. This was followed by the addition of 50 μl recombinant human TNFα (R&D Systems) to obtain a final assay concentration of 20 ng/ml and a subsequent 2 h incubation at 37° C. Following the incubation period, 50 μl of Caspase 3 assay reaction buffer (0.2% w/v CHAPS, 0.5% v/v Igepal CA-630, 200 mM NaCl, 50 mM HEPES, 20 μM DEVD-R110 substrate (Invitrogen)) was added and cells incubated for 2.5 h at 37° C. Fluorescence was measured by excitation at 475 nm and emission 512 nm. Caspase activity in the absence of a TNFα antagonist is shown in FIG. 8A.

The functional activity of TNFR2-Fc_VH #4 was determined using a TNFα induced Caspase 3 activity assay in U937 cells. TNFR2-Fc_VH #4 completely inhibited TNFα induced Caspase 3 activity as did etanercept (FIG. 8B). This clearly illustrates that the TNFα inhibitory portion of TNFR2-Fc_VH #4 is biologically active and has a similar potency to etanercept. Similar data was also observed for TNFR2-Fc_varB and ndimab varB (see FIG. 8C).

### Example 6—In Vivo Assays

All in vivo procedures were carried out in accordance with the UK Home Office Animals (Scientific Procedures) Act (1986) and approved by a local ethics committee. Female C57Bl/6 mice (Charles River, UK) were used throughout. Mice were housed in groups of ⅚ per cage, in individually ventilated cages (IVC) with free access to food and water under a 12-hour light/dark cycle (lights on 07:00-19:00). Housing and procedure rooms were maintained at 24° C. and constant background noise was maintained by way of a conventional radio station. All mice underwent insertion of transponders under anaesthesia (3% isoflurane in oxygen) for identification purposes at least 5 days before the start of each study.

### A. Seltzer Model of Neuropathic Pain

Mechanical hyperalgesia was determined using an analgysemeter (Randall L O, Selitto J J, Arch Int Pharmacodyn Ther. 111:409-19 (1957)) (Ugo Basile). An increasing force was applied to the dorsal surface of each hind paw in turn until a withdrawal response was observed. The application of force was halted at this point and the weight in grams recorded. Data was expressed as withdrawal threshold in grams for ipsilateral and contralateral paws. Following the establishment of baseline readings mice were divided into 2 groups with approximately equal ipsilateral/contralateral ratios and underwent surgery. Mice were anaesthetised with 3% isoflurane. Following this approximately 1 cm of the left sciatic nerve was exposed by blunt dissection through an incision at the level of the mid thigh. A suture (10/0 Virgin Silk: Ethicon) was then passed through the dorsal third of the nerve and tied tightly. The incision was closed using glue and the mice were allowed to recover for at least seven days prior to commencement of testing Sham operated mice underwent the same protocol but following exposure of the nerve the wound was glued and allowed to recover. Mice were tested for hyperalgesia on day 7 and 10 post surgery. Following testing on day 10, operated mice were further sub-divided into groups which received CAT251 IgG1 isotype control (0.03 mg/kg s.c.), etanercept (0.01 mg/kg s.c.), MEDI-578 (0.03 mg/kg s.c.) or a combination of etanercept (0.01 mg/kg s.c.) and MEDI-578 (0.03 mg/kg s.c.). Sham operated mice all received CAT251 (0.03 mg/kg s.c.). Mechanical hyperalgesia was measured at 4 h, 1, 2, 3, 4 and 7 days post dose.

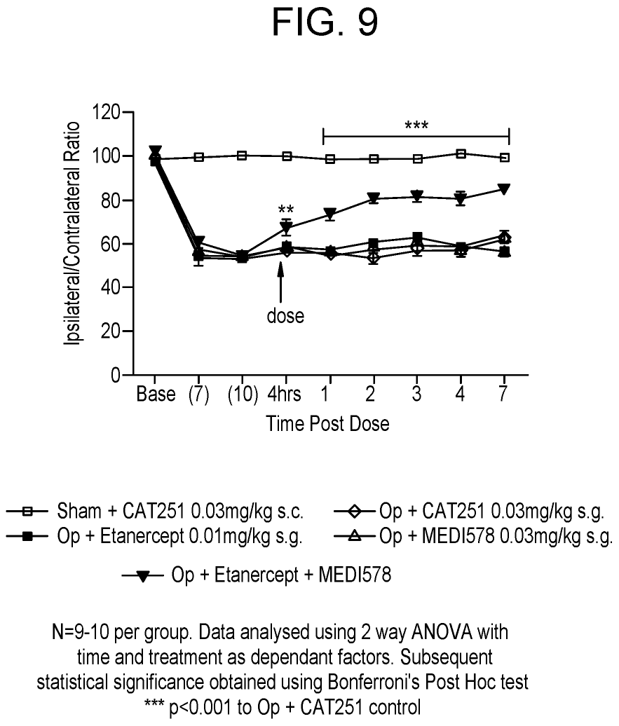

Co-administration of etanercept and MEDI-578 in a mechanical hyperalgesia model manifested as a significant reduction in the ipsilateral/contralateral ratio on day post surgery when compared to sham operated controls (FIG. 9). Administration of a single dose of either etanercept (0.01 mg/kg s.c.) or MEDI-578 (0.03 mg/kg s.c.) failed to significantly reverse this hyperalgesia. The co-administration of etanercept (0.01 mg/kg s.c.) together with MEDI-578 (0.03 mg/kg s.c.) significantly reversed the mechanical hyperalgesia at 4 h post dose and the effect was maintained through to 7 days post dose.

In a second study the effect of TNFR2-Fc_VH #4 was assessed. Following establishment of a mechanical hyperalgesia, mice were dosed on day 13 post surgery with R347 Bs3Ab isotype control (0.03 mg/kg s.c.), etanercept (0.01 mg/kg s.c.), MEDI-578 (0.03 mg/kg s.c.) or TNFR2-Fc_VH #4 (0.01 mg/kg or 0.03 mg/kg s c) Sham prepared animals received R347 Bs3Ab isotype control (0.03 mg/kg s.c.). Mice were tested for mechanical hyperalgesia at 4 h post dose and on days 1, 2, 4 and 7 post dose as described above.

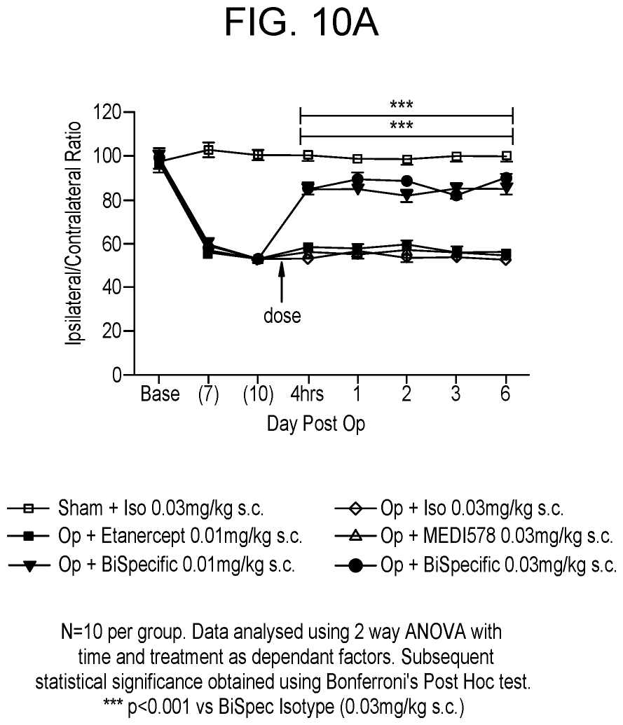

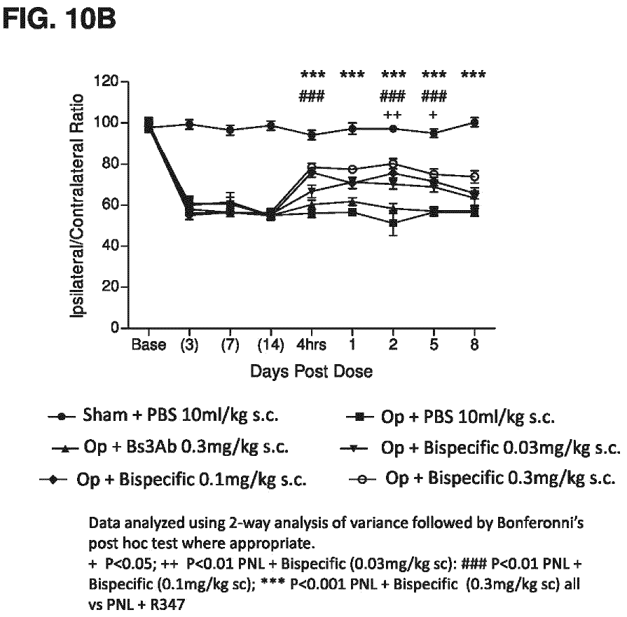

Administration of TNFR2-Fc_VH #4 produced a significant reduction in the ipsilateral/contralateral ratio on day 10 post surgery when compared to sham operated controls (FIG. 10A). The administration of either etanercept (0.01 mg/kg s.c.) or MEDI-578 (0.03 mg/kg s.c.) failed to significantly reverse the mechanical hyperalgesia. However, the administration of TNFR2-Fc_VH #4 (0.01 and 0.03 mg/kg s.c.) produced a significant reversal of the mechanical hyperalgesia at 4 h post dose, an effect which was maintained through to 6 days post dose. No effect was seen following administration of the R347 control Bs3Ab. Similar data was observed when TNFR2-Fc_varB was administered (see FIG. 10B). These data suggest that TNFR2-Fc_VH #4 can significantly reverse pain at very low doses where equivalent doses have been shown to be ineffective or minimally effective with either MEDI-578 or etanercept alone.

### B. Chronic Joint Pain Model

Mechanical hypersensitivity was determined using a mouse incapacitance tester (Linton Instrumentation). Mice were placed in the device with their hind paws on separate sensors, and the body weight distribution calculated over a period of 4 s. Data was expressed as the ratio of ipsilateral and contralateral weight bearing in grams.

Following the establishment of baseline readings, mice were divided into 2 groups with approximately equal ipsilateral/contralateral ratios. Intra-articular injections were carried out using the following technique: animals were anesthetised using 3% isoflurane in oxygen and the left knee was shaved and cleaned. The knee joint of each mouse was injected with either 10 μl of Freund's complete adjuvant (FCA) (10 mg/ml) or vehicle (light mineral oil) using a 25-gauge needle mounted on a 100 μl Hamilton syringe. Injections were made directly into the synovial space of the knee joint. Mice were allowed to recover and were re-tested for changes in mechanical hypersensitivity on days 7 and 10 post injection as described above. Following testing on day 10, FCA treated mice were further randomised into groups and on day 13 mice were dosed with etanercept (0.01 mg/kg i.p.) or vehicle after which they received a dose of MEDI-578 (0.03 mg/kg i.v.) or CAT251 isotype control (0.03 mg/kg i.v.). Mice were tested for mechanical hypersensitivity at 4 h post dose and on days 1, 2, 4 and 7 post dose as described above.

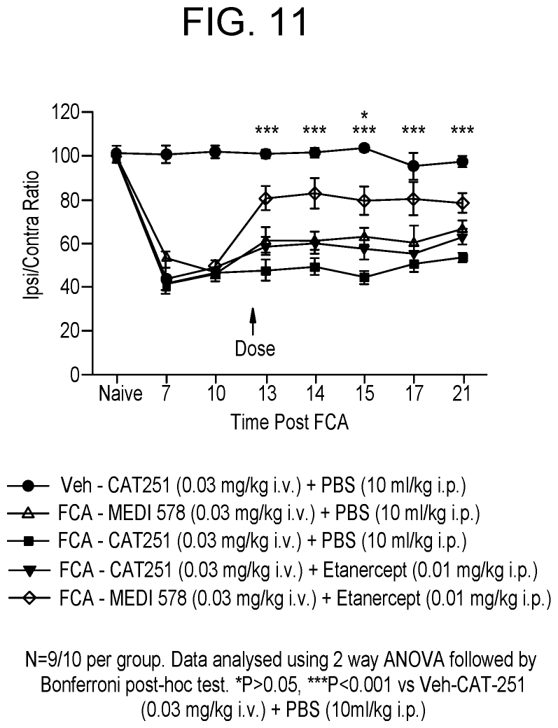

The effect of co-administration of etanercept and MEDI-578 was assessed using the intra-articular FCA model of inflammatory pain. Intra-articular administration of FCA caused a mechanical hypersensitivity that manifested as a significant reduction in the ipsilateral/contralateral ratio on days 7 and 10 when compared to vehicle control (FIG. 11). No reduction in the ipsilateral/contralateral ratio was observed in the sham treated groups compared to pre-treatment baseline levels. The administration of etanercept (0.01 mg/kg i.p.)+CAT251 (0.03 mg/kg i.v.) or PBS (10 ml/kg i.p.)+MEDI-578 (0.03 mg/kg i.v.) caused a slight reversal of the FCA induced mechanical hypersensitivity at 4 h and days 1, 2, 4 and 7 post dose but this failed to reach statistical significance. However, the administration of etanercept (0.01 mg/kg i.p.)+MEDI-578 (0.03 mg/kg i.v.) caused a significant reversal of the FCA induced mechanical hypersensitivity at all times of testing post dose.

In a second study, the effect of TNFR2-Fc_VH #4 was assessed. Following establishment of FCA induced mechanical hypersensitivity, mice were dosed on day 13 post-FCA with: R347 Bs3Ab isotype control (0.01 mg/kg s.c.), etanercept (0.01 mg/kg s.c.), MEDI-578 (0.01 mg/kg s.c.) or TNFR2-Fc_VH #4 (0.003 mg/kg or 0.01 mg/kg s.c.). Again mice were tested for mechanical hypersensitivity at 4 h post dose and on days 1, 2, 4 and 7 post dose as described above.

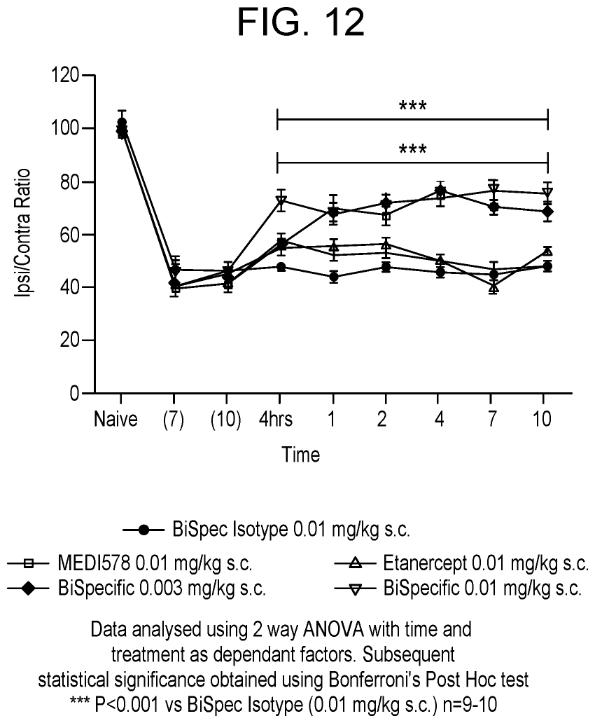

The effect of TNFR2-Fc_VH #4 (“bispecific”) as compared to the effects of etanercept and MEDI-578 individually is shown in FIG. 12. Neither etanercept (0.01 mg/kg s.c.) nor MEDI-578 (0.01 mg/kg s.c.) significantly reversed the FCA induced mechanical hypersensitivity at any time point post dose. However, administration of TNFR2-Fc_VH #4 caused a significant reversal of FCA induced mechanical hypersensitivity. The higher dose of TNFR2-Fc_VH #4 (0.01 mg/kg s.c) showed significant activity for the duration of the study whereas the lower dose (0.003 mg/kg s.c.) reached significance on day 1 post dose and remained at a similar level to the higher dose for the duration of the study.

### C. Established FCA Induced Model of Mechanical Hypersensitivity in the Rat

Intraplantar injection of Freunds Complete adjuvant (FCA) causes an inflammatory reaction, which induces hypersensitivity and edema, and mimics some aspects of clinical inflammatory pain. These effects can be investigated using equipment to measure weight bearing. Assessment of potential anti-hyperalgesic properties of TNFR2-Fc_VH #4 FCA induced hypersensitivity using weight bearing method. Naive rats distribute their body weight equally between the two hind paws. However, when the injected (left) hind paw is inflamed and/or painful, the weight is re-distributed so that less weight is put on the affected paw (decrease in weight bearing on injured paw). Weight bearing through each hind limb is measured using a rat incapacitance tester (Linton Instruments, UK). Rats are placed in the incapacitance tester with the hind paws on separate sensors and the average force exerted by both hind limbs are recorded over 4 seconds.

For this study, naïve rats (Male, Sprague Dawley Rats (Harlan, UK), 198-258 g) were acclimatised to the procedure room in their home cages, with food and water available ad libitum. Habituation to the incapacitance tester was performed over several days. Baseline weight bearing recordings were taken prior to induction of insult. Inflammatory hypersensitivity was induced by intraplantar injection of FCA (available from Sigma, 100 μl of 1 mg/ml solution) into the left hind paw. A pre-treatment weight bearing measurement was taken to assess hypersensitivity 23 hours post-FCA.

Animals were then ranked and randomised to treatment groups according to the weight bearing FCA window in a Latin square design. At 24 hours post FCA injection, animals were treated with either TNFR2-Fc_VH #4 (“bispecific”) given i.v. at 0.003, 0.03, 0.3, & 3 mg/kg, a negative control antibody, NIP228 (an antibody raised to bind to hapten nitrophenol) given i.v. at 3 mg/kg, vehicle (1% Methylcellulose) given p.o. 2 ml/kg, or indomethacin given 10 mg/kg p.o.

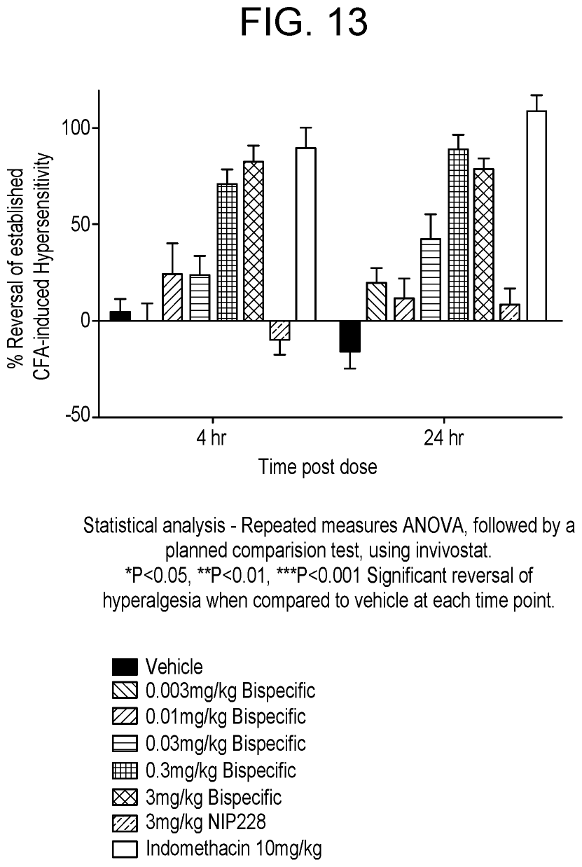

Weight bearing was assessed 4 and 24 hours post antibody/drug treatment. Data were analyzed by comparing treatment groups to the vehicle control group at each time point. Statistical analysis included repeated measures ANOVA followed by Planned comparison test using InVivoStat (invivostat.co.uk), (p<0.05 considered significant). The results are shown in FIG. 13. A significant reversal of the hypersensitivity was observed with Indomethacin (10 mg/kg) at 4 and 24 hours. TNFR2-Fc_VH #4 dosed at 0.3 and 3 mg/kg showed significant reversal of the hypersensitivity at both 4 and 24 hours, TNFR2-Fc_VH #4 dosed at 0.003 and 0.03 mg/kg also showed a significant reversal of the hypersensitivity, but only at 24 hours. The isotype control, NIP228 had no significant effect on the FCA response at any time point.

### Example 7— p38 Phosphorylation by TNFα and NGF

Literature suggests that p38 phosphorylation plays an important role in the development of neuropathic pain. For example, treatment with p38 inhibitors have been shown to prevent the development of neuropathic pain symptoms in the spared nerve injury model (Wen Y R et al., Anesthesiology 2007, 107:312-321) and in a sciatic inflammatory neuropathy model (Milligan E D et al., J Neurosci 2003, 23:1026-1040). In the present experiment, the role of TNFα, NGF, and the combination TNFα and NGF on p38 phorphorylation was investigated in a cell culture assay. Briefly, Neuroscreen-1 cells (a subclone of PC-12 rat neuroendocrine cells) were incubated with increasing amounts of TNFα, NGF, or a combination of TNFα and NGF. Following a 20 minute incubation period, phospho-p38 was quantified using a homogeneous time resolved fluorescence (HTRF) assay (Cisbio).

HTRF Assay: Following stimulation with TNFα, NGF, or a combination of TNFα and NGF, cell supernatants were rapidly removed and cells lysed in lysis buffer. Phospho-p38 MAPK (Thr180/Tyr182) was detected in lysates in a sandwich assay format using two different specific antibodies; an anti-phospho-p38 antibody conjugated to europium cryptate (donor fluorophore) and an anti-p38 (total) antibody conjugated to d2 (acceptor fluorophore). Antibodies were incubated with cell lysates and HTRF ratios calculated from fluorescence measurements at 665 nm and 620 nm made using an EnVision Multilabel Plate Reader (Perkin Elmer).

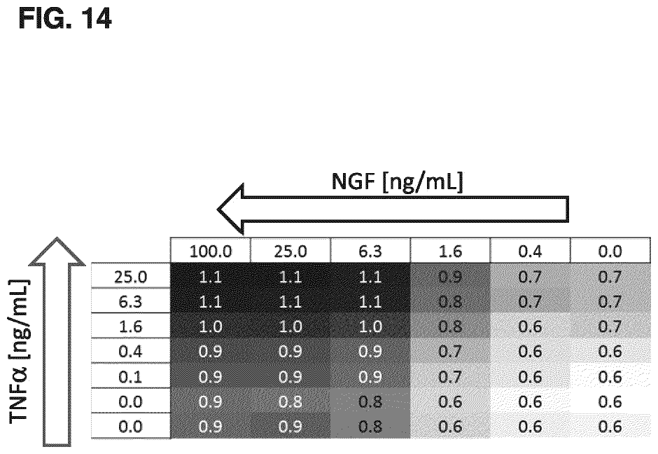

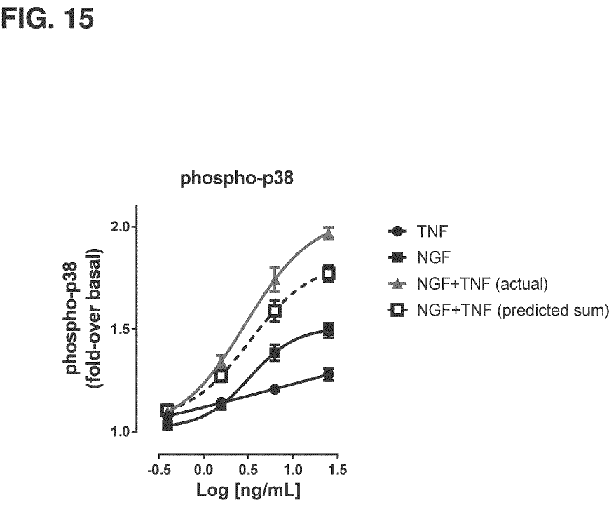

Data are presented as HTRF ratios, which are calculated as the ratio between the emission at 665 nm and the emission at 620 nm. A heat map showing HTRF ratios from phospho-p38 reactions is shown in FIG. 14. Dose response curves showing the effect of TNFα, NGF, or a combination of TNFα and NGF are shown in FIG. 15. As can be seen from FIG. 15, the combined effect of higher concentrations of TNFα and NGF on phospho-p38 is greater than the predicted sum of the phospho-p38 signal induced by either factor alone. These data suggest that TNFα and NGF may act together to induce p38 phosphorylation, and that the two pathways may be implicated in molecular signaling leading to pain.

### Example 8— ERK Phosphorylation by TNFα and NGF

Like p38, ERK is also activated during neuropathic pain development (Zhuang Z Y et al., Pain 2005, 114:149-159). In the present experiment, the role of TNFα, NGF, and the combination TNFα and NGF on ERK phorphorylation was investigated in a cell culture assay. Briefly, Neuroscreen-1 cells (a subclone of PC-12 rat neuroendocrine cells) were incubated with increasing amounts of TNFα, NGF, or a combination of TNFα and NGF. Following a 20 minute incubation period, phospho-ERK was quantified using a HTRF assay (Cisbio).

HTRF Assay: Following stimulation, cell supernatants were rapidly removed and cells lysed in lysis buffer. Phospho-ERK MAPK (Thr202/Tyr204) was detected in lysates in a sandwich assay format using two different specific antibodies; an anti-phospho-ERK antibody conjugated to europium cryptate (donor fluorophore) and an anti-ERK (total) antibody conjugated to d2 (acceptor fluorophore). Antibodies were incubated with cell lysates and HTRF ratios calculated from fluorescence measurements at 665 nm and 620 nm made using an EnVision Multilabel Plate Reader (Perkin Elmer).

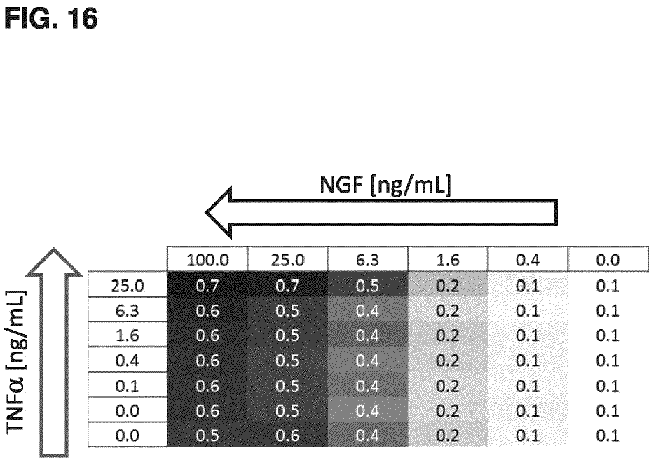

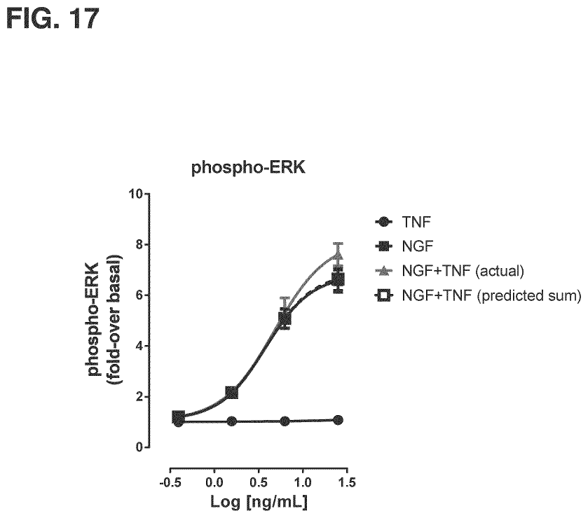

Data are presented as HTRF ratios, which are calculated as the ratio between the emission at 665 nm and the emission at 620 nm. A heat map showing HTRF ratios from phospho-ERK reactions is shown in FIG. 16. Dose response curves showing the effect of TNFα, NGF, or a combination of TNFα and NGF are shown in FIG. 17. As can be seen from FIG. 17, low amounts of TNFα alone did not induce phospho-ERK, but higher amounts, enhanced NGF-induced phospho-ERK. These data suggest that TNFα and NGF may act together to induce p38 phosphorylation, and that the two pathways may be implicated in molecular signaling leading to pain.

### Example 9—Effects of Different Doses of TNFR2-Fc_varB in Humans with Painful Osteoarthritis of the Knee

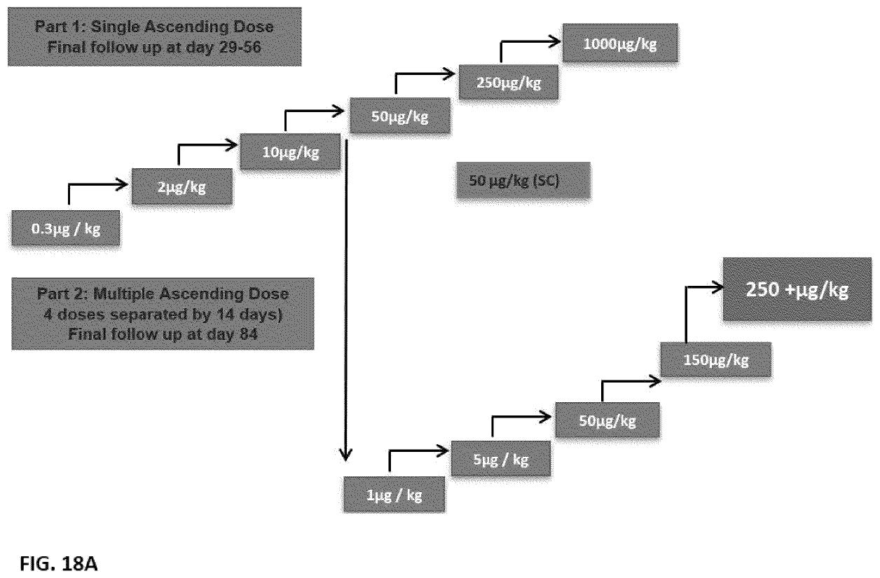

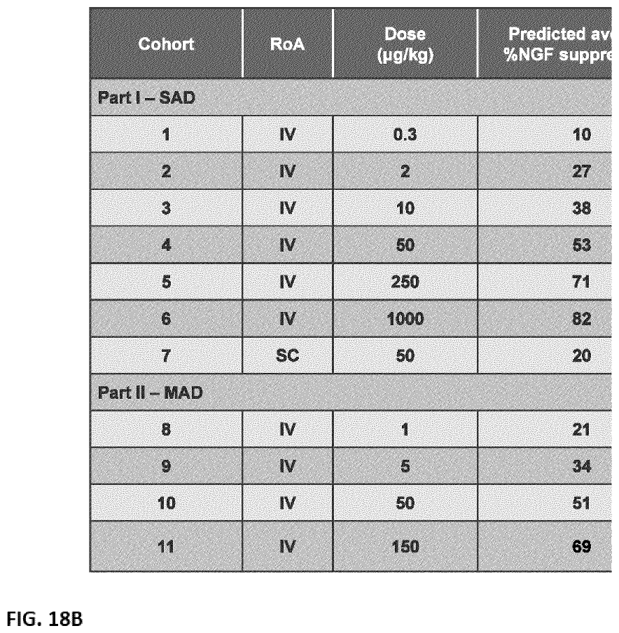

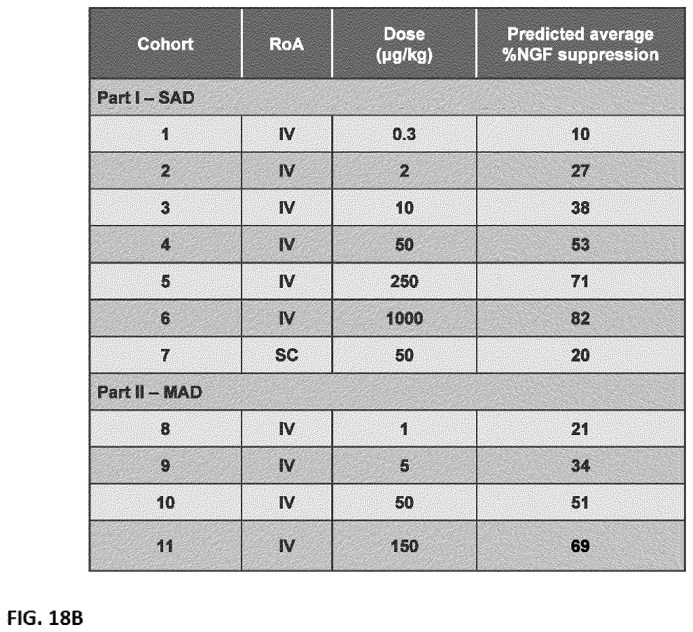

A multi-center, randomized, double-blind, placebo-controlled, interleaved single-ascending dose (SAD) and multiple-ascending dose (MAD) study was designed for subjects 18 to 80 years of age, with painful osteoarthritis of the knee. The SAD cohort 1 included three patients receiving TNFR2-Fc_varB and 2 receiving placebo. The SAD cohorts 2-7 included 8 patients each, with six in each cohort receiving TNFR2-Fc_varB and 2 in each cohort receiving placebo. The MAD cohorts 8 and 9 included 18 patients each, with 12 in each cohort receiving TNFR2-Fc_varB and 6 in each cohort receiving placebo. The MAD cohorts 10 and 11 included 12 patients each, with 9 in each cohort receiving TNFR2-Fc_varB and 3 in each cohort receiving placebo. A simplified layout of the study design is provided in FIGS. 18A and 18B.

Subjects in the SAD cohorts received either a single infusion of TNFR2-Fc_varB or placebo in a double-blind manner Following discharge, subjects were instructed to record pain daily on an 11-point NRS (0-10) at approximately the same time each morning, to reflect 24 hours of recall, to the end of the follow-up period.

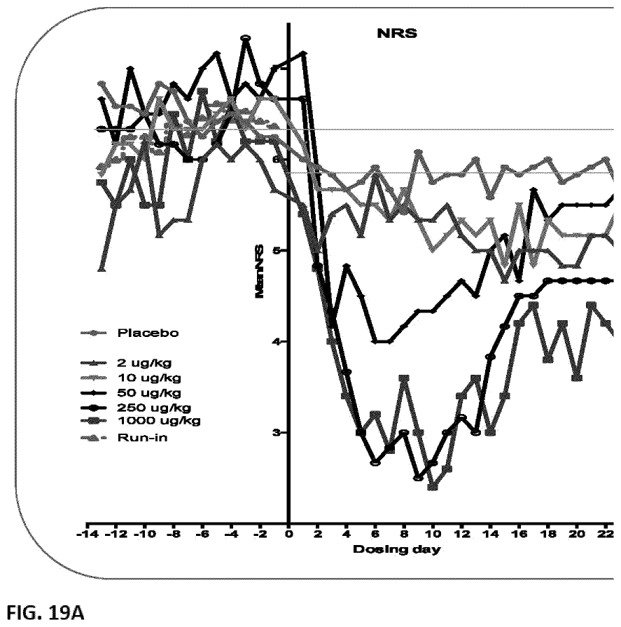

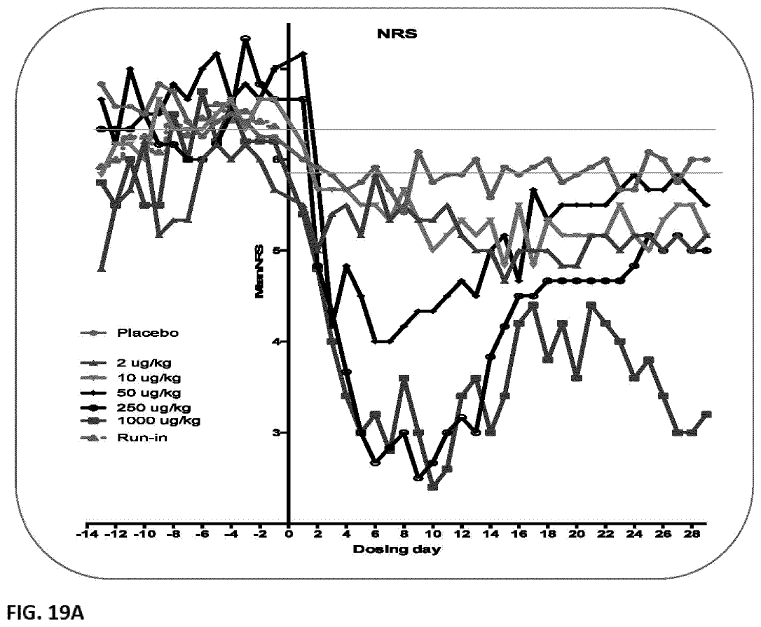

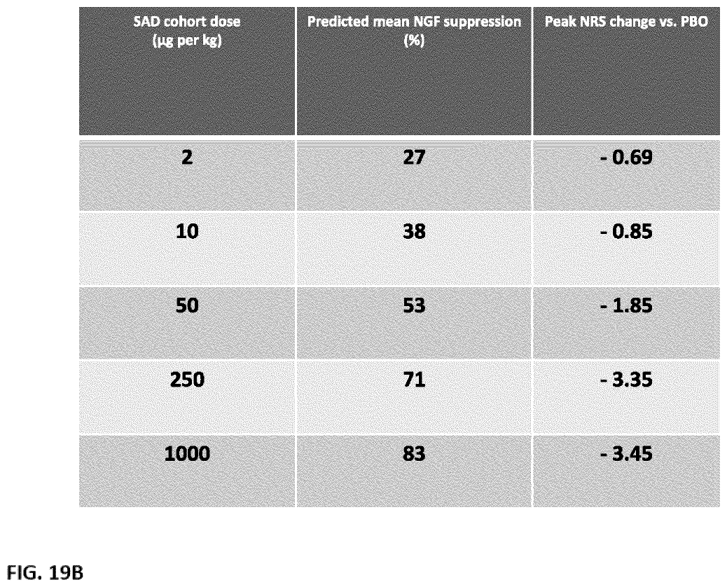

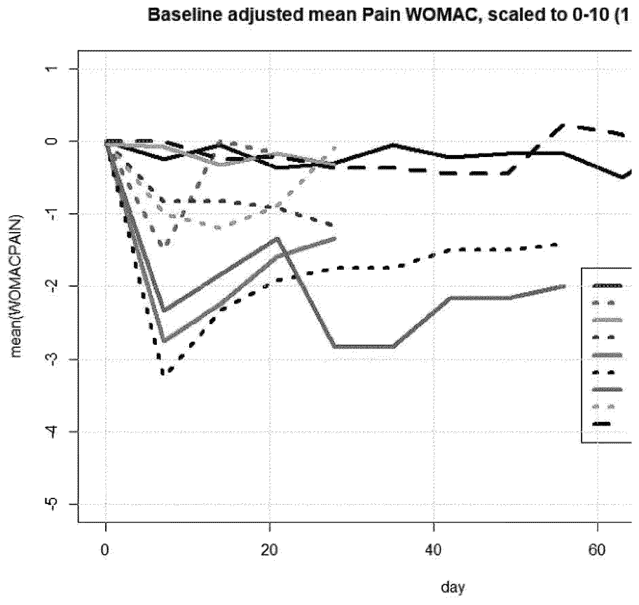

Surprisingly, a single intravenous dose of TNFR2-Fc_varB ranging in dose from 2 to 1000 lag/kg appeared to reverse the daily average pain score (at peak effect) by 0.69 to 3.45 points vs. placebo (FIGS. 19A-19B). This effect is statistically significant (p≤0.01) at doses of 50, 250 and 1000 lag/kg. The duration of this effect surprisingly lasted more than 10 days as compared with the half-life of TNFR2-Fc_varB (3-4 days). The decrease in pain score for those subjects receiving placebo appears to be approximately 0.5 points. This placebo effect is relatively low and appears stable.

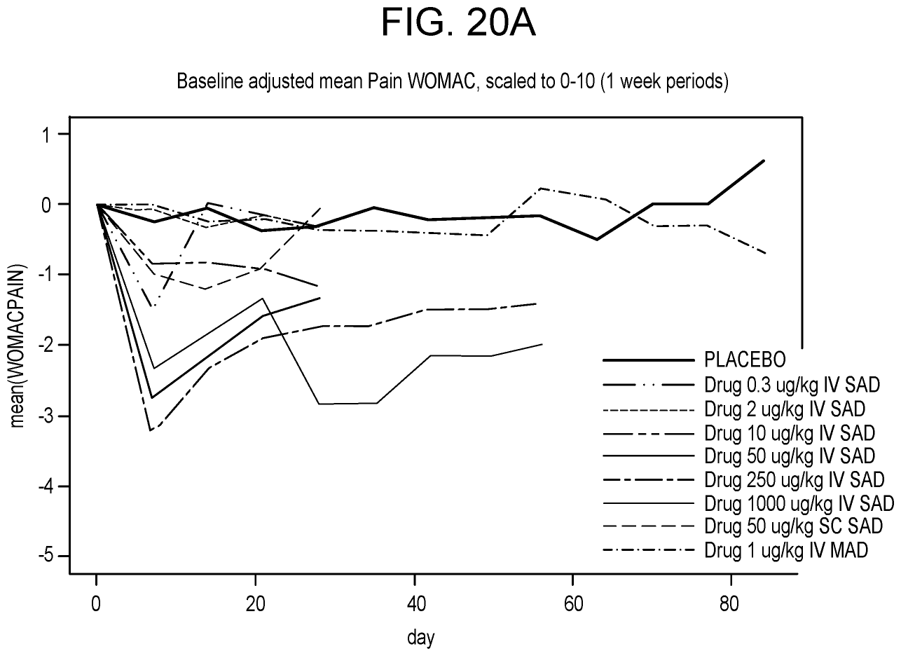

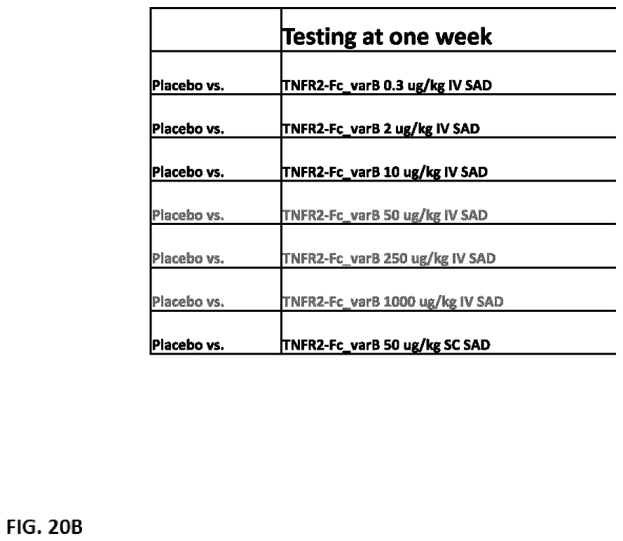

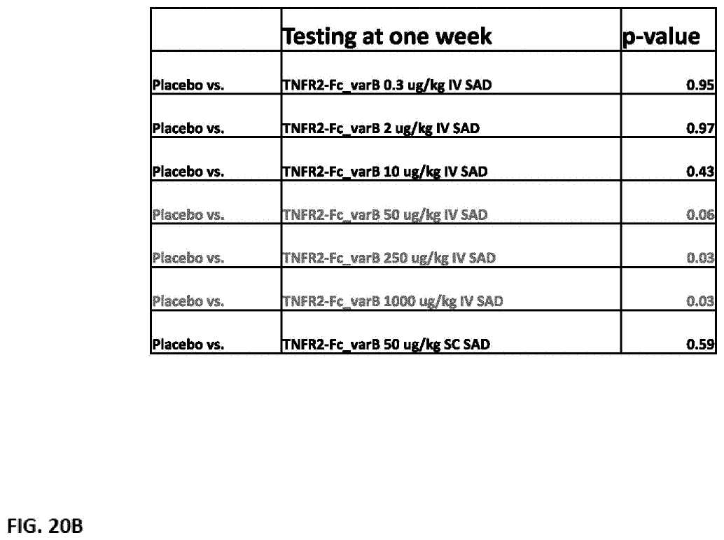

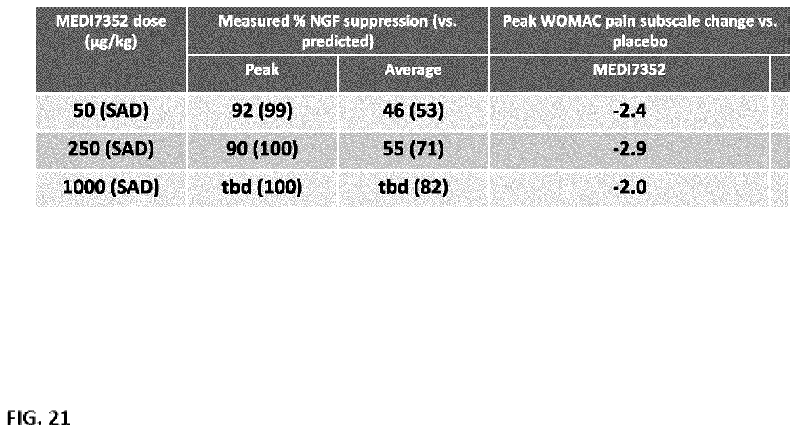

The Western Ontario and McMasters Universities osteoarthritis index (WOMAC) is a questionnaire based tool to measure functional impairment as a result of chronic pain in subjects with OA. Surprisingly, single administrations of TNFR2-Fc_varB at doses ranging from 0.3 to 1000 lag/kg significantly decreased the mean WOMAC pain subscale score over a period of 10+ days by up to −3 points (FIGS. 20A-20B). At doses of 50, 250 and 1000 lag/kg the peak reversal of the pain subscale score ranges from 2.0-2.9 and is statistically significant with p values of 0.06 or less. As with the pain NRS endpoint the duration of effect after a single dose (˜10+ days) was longer than anticipated for a molecule with a half-life of 3-4 days. Peak effect corresponded with measured suppression of free NGF of 46-55% at doses of 50 and 250 lag/kg, respectively (FIG. 21).

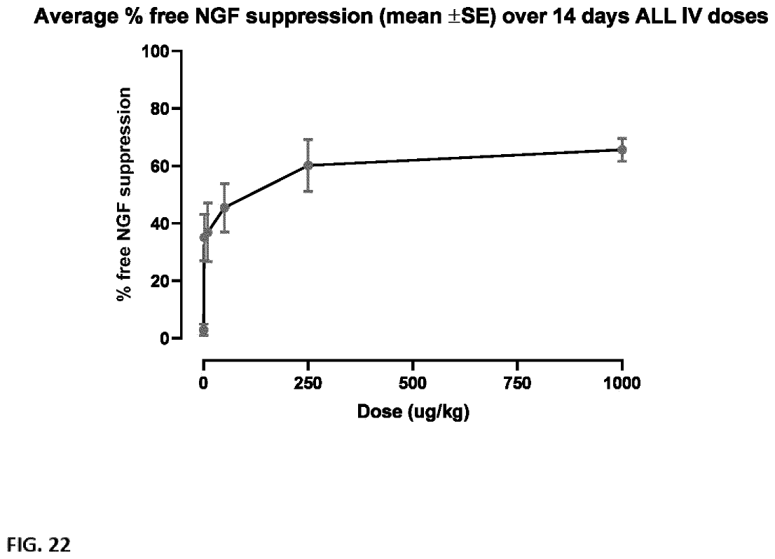

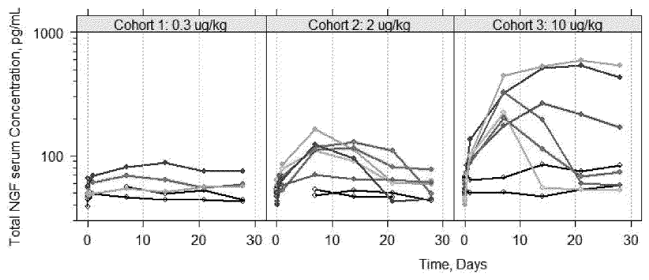

The effect of TNFR2-Fc_varB on levels of free NGF in the periphery was determined using a Singulex Erenna assay. Briefly, blood samples were taken from each subject at timepoints pre-dose, 1, 8 and 24 hours post-dose, days 8, 15, 22, 29 (days 43 and 56 for the two highest doses only). Plasma samples were prepared and assayed according to the following steps (1) mix samples with anti NGF mAb coated magnetic beads, (2) captured NGF magnetic bead complex is mixed with a fluorescently labelled anti-human NGF antibody, (3) elution of bead complex to release fluorescent labels, (4) fluorescent signal read in an Erenna fluorescence reader. Suppression of free NGF was calculated and the average suppression over the 14 day period post dose at each concentration of TNFR2-Fc_varB was calculated and plotted (FIG. 22). Average suppression of free NGF over 14 days ranged from 0 (0.3 lag/kg) to −65% (1000 lag/kg).

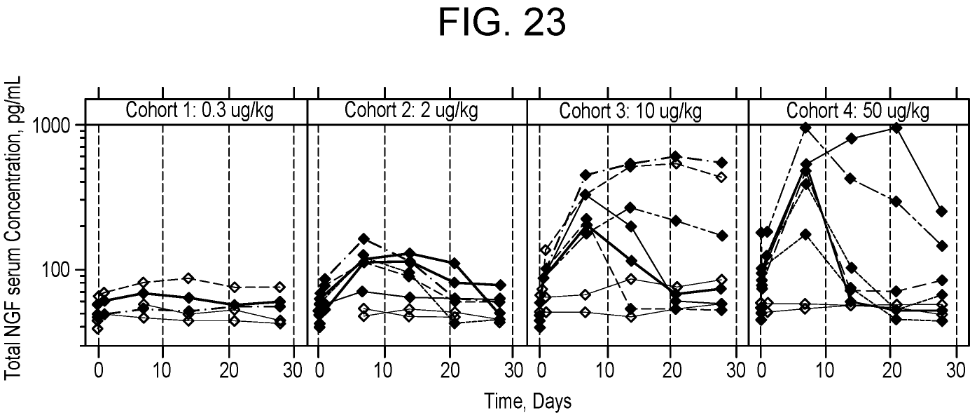

The effect of TNFR2-Fc_varB on levels of total NGF in the periphery was determined using a LC-MS/MS assay developed by Q2 Solutions. Briefly, blood samples were taken from each subject at timepoints pre-dose, 1, 8 and 24 hours post dose, days 8, 15, 22, 29 (days 43 and 56 for the two highest doses only). Serum samples were prepared and assayed in a manner similar to that described in Neubert et al., 2013, Anal. Chem., 85:1719-1726. Increases in total NGF levels were calculated and plotted for each subject in SAD cohorts 1-4 (0.3-50 lag/kg) and average total NGF levels were calculated for each of cohorts 1-7. A clear increase in levels of total NGF was observed after increased doses of a single administration of TNFR2-Fc_varB (FIG. 23; Table 2). Without wishing to be bound by theory, the increase may be due to the half-life of NGF increasing in line with that of TNFR2-Fc_varB to which it is now bound.

TABLE 2
Average levels of total NGF in the periphery
after treatment with TNFR2-Fc_varB

| | | | Dose | Observed average | Average total |
|---|---|---|---|---|---|
| Cohort | N | RoA | (μg/kg) | % NGF suppression | NGF (pg/mL) |
| 1 | 3 | IV | 0.3 | 3 | 65.2 |
| 2 | 6 | IV | 2 | 27 | 98.1 |
| 3 | 6 | IV | 10 | 29 | 228.0 |
| 4 | 6 | IV | 50 | 35 | 334.0 |
| 5 | 6 | IV | 250 | 59 | 539.0 |
| 6 | 6 | IV | 1000 | 68 | 199.0* |
| 7 | 6 | SC | 50 | 37 | 206.0 |

*only 1 subject data available.
RoA Route of administration,
IV = intravenous,
SC = subcutaneous

It should be noted that there was no apparent increase in total NGF for two subjects in each cohort. As the study remains blinded at the time of filing of this application, the prediction is that these are placebo samples. For cohorts 3 and 4 there was observed an apparent effect on the total NGF levels of anti-drug antibodies. This effect was likely due to a decrease in exposure of TNFR2-Fc_varB and a corresponding shortening of the duration of effect.

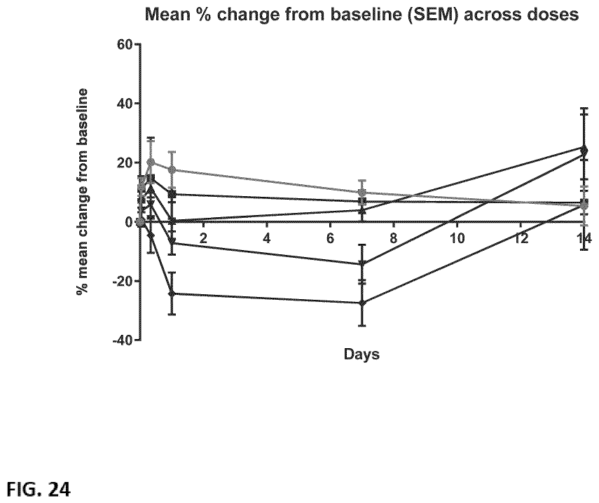

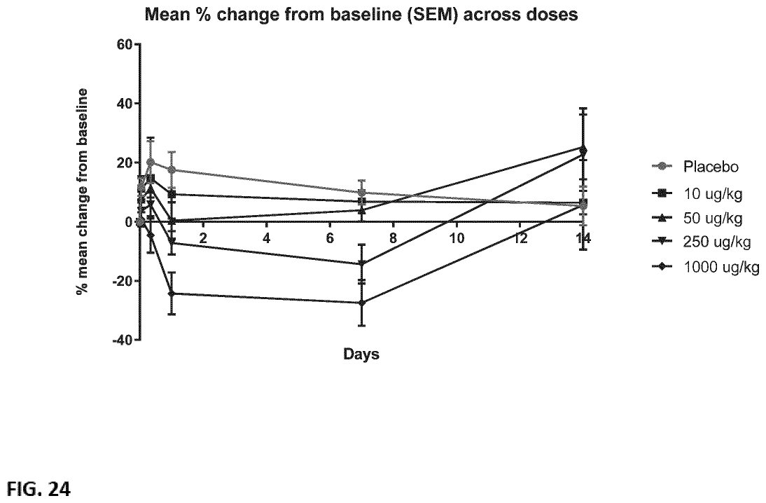

As a proxy for measuring levels of TNFα levels, CXCL-13 levels may be measured using the Simoa platform technology. CXCL-13 gene expression is regulated by the lymphotoxin alpha pathway. TNFR2-Fc_varB binds TNFα and lymphotoxin alpha, and as such was hypothesized to have an effect on levels of CXCL-13 expression. Blood samples were taken from each subject at timepoints pre-dose, 1, 8 and 24 hours post dose, days 8, 15, 22, 29 (days 43 and 56 for the two highest doses only). Serum samples were prepared and then assayed in the Simoa CXCL-13 assay. A clear dose response was observed with increasing suppression of CXCL-13 levels observed after administration of increasing single doses of TNFR2-Fc_varB (FIG. 24).

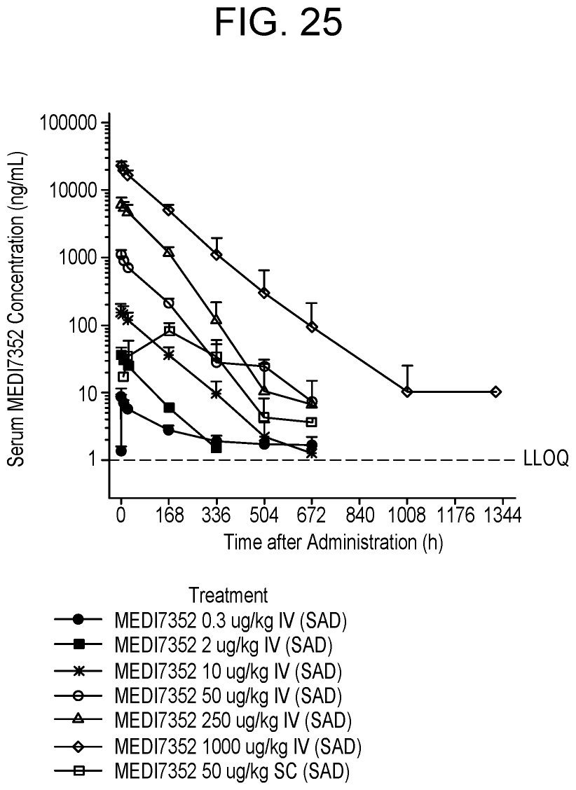

Serum levels of TNFR2-Fc_varB administered intravenously were determined at various time intervals following single ascending doses. The observed serum pharmacokinetics of TNFR2-Fc_varB indicated that all cohorts had exposure, and exposures increased in a dose-dependent manner on average (FIG. 25). FIG. 25 shows that subcutaneous administration provided a relatively stable serum level of TNFR2-Fc_varB for over 10 days.

Absolute bioavailability via subcutaneous administration was calculated by comparing the geometric mean values (n=6) of area under the curve (AUC) from single doses of 50 μg/kg subcutaneous versus 50 μg/kg intravenous administration of TNFR2-Fc_varB as shown in Table 3. The bioavailability of subcutaneous administration of TNFR2-Fc_varB was found to be surprisingly low and was estimated to be 21%. As shown in Table 3a, the 90% confidence interval for the estimated absolute bioavailability value was 0.1627 to 0.2781.

TABLE 3
Preliminary analysis of pharmacokinetics of intravenous
and subcutaneous administration of TNFR2-Fc_varB

| | | TNFR2-Fc_varB | |
|---|---|---|---|
| | | dose (route) | |
| Parameter | | 50 μg/kg | 50 μg/kg |
| (units) | Statistic | (iv) | (sc) |
| Cₘₐₓ (ng/ml) | N | 6 | 6 |
| | Geometric mean | 1082 | 78.53 |
| | CV (%) | 14.04 | 32.1 |
| tₘₐₓ (days) | N | 6 | 6 |
| | Median | 0.04 | 7.07 |
| | Min, max | 0.04, 0.04 | 7.01, 8.09 |
| tₗₐₛₜ (days) | N | 6 | 6 |
| | Median | 10.473 | 17.622 |
| | Min, max | 6.95, 27.90 | 14.01, 29.10 |
| AUCₗₐₛₜ (days · ng/ml) | N | 6 | 6 |
| | Geometric mean | 3766 | 801 |
| | CV (%) | 26.87 | 25.16 |
| AUC_{0-∞} (days · ng/ml) | N | 6 | 1 |
| | Geometric mean | 4267 | NC |
| | CV (%) | 17.89 | NC |
| t_{1/2} (days) | N | 6 | 1 |
| | Arithmetic mean | 3.304 | NC |
| | SD | 0.5104 | NC |
| Vₛₛ (L)^{a} | N | 6 | 1 |
| | Arithmetic mean | 4.473 | NC |
| | SD | 0.3705 | NC |
| CL (L/day)^{a} | N | 6 | 1 |
| | Arithmetic mean | 0.9649 | NC |
| | SD | 0.1582 | NC |

If N<3, summary statistics were not calculated.

AUC_{0-∞} Area under the concentration-time curve from zero to infinity; AUCₗₐₛₜ Area under the concentration-time curve from zero to the last quantifiable timepoint; CL Clearance; Cₘₐₓ Maximum observed concentration; CV Coefficient of variation (geometric); iv Intravenous; Max Maximum; min Minimum; N Number of subjects; sc Subcutaneous; NC Not calculable; SD Standard deviation; t_{1/2} Half-life; tₗₐₛₜ Time of last observed quantifiable concentration; tₘₐₓ Time to Cₘₐₓ; Vₛₛ Volume of distribution at steady state.

TABLE 3a
Absolute bioavailability analysis of TNFR2-
Fc_varB via subcutaneous administration

| | | | | | Comparison of SC and | |
|---|---|---|---|---|---|---|
| | | | | | IV administration (ratio | |
| | Dose and | | | | of geometric LS means) | |
| Parameter | route of | | | Geometric | Ratio | 90% |
| (units) | administration | | n | LS mean | (SC:IV) | CI |
| Cₘₐₓ (ng/mL) | 50 | μg/kg SC | 6 | 78.53 | 0.0725 | 0.0563, |
| | 50 | μg/kg IV | 6 | 1082.47 | | 0.0935 |
| AUCₗₐₛₜ | 50 | μg/kg SC | 6 | 801.17 | 0.2127 | 0.1627, |
| (days · ng/mL) | 50 | μg/kg IV | 6 | 3766.28 | | 0.2781 |

AUCₗₐₛₜ area under the concentration-time curve from zero to the last quantifiable time point; Cₘₐₓ maximum observed concentration; IV intravenous; LS least squares; n number of subjects included in the analysis; PK pharmacokinetic; SC subcutaneous.

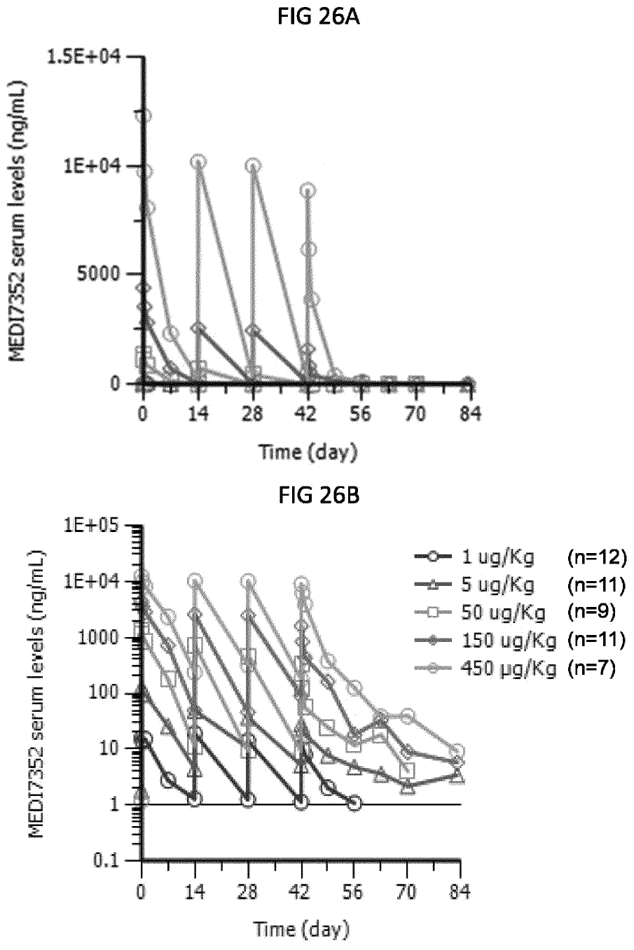

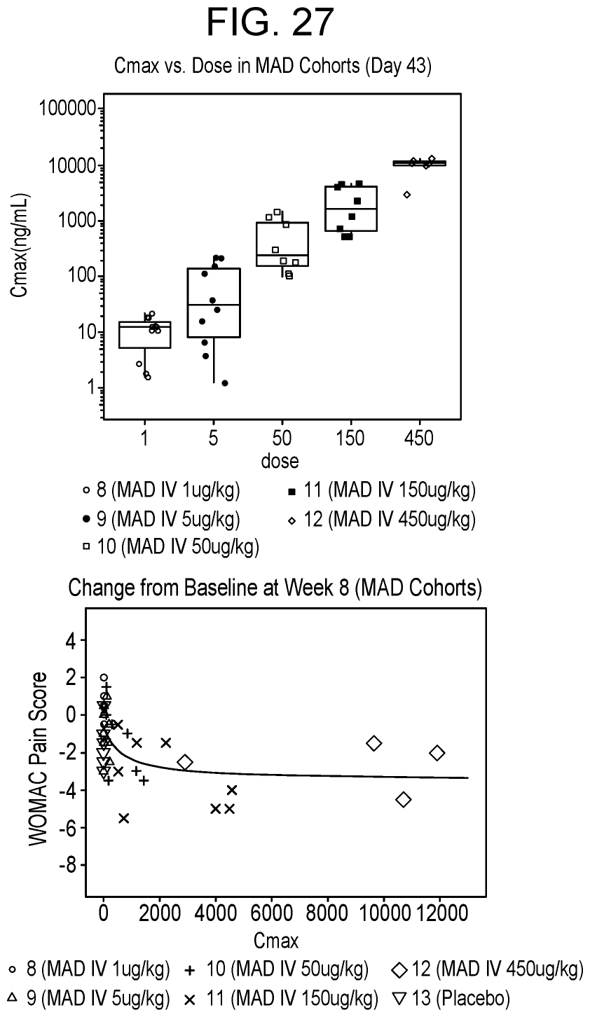

Subjects in the MAD cohorts received repeated intravenous infusions of TNFR2-Fc_varB ranging from 1 to 450 μg/kg every 2 weeks (4 doses in total) or placebo in a double-blind manner. The observed serum pharmacokinetics of TNFR2-Fc_varB indicated that all cohorts had exposure, and exposures increased in a dose-dependent manner on average (FIG. 26). Exposure-response analysis suggests that the maximum pain reduction efficacy was reached in the 150-450 μg/kg intravenous dose range (FIG. 27; Table 4).

TABLE 4
NGF and pain response in subjects treated with TNFR2-Fc_varB

| | | | | Observed | WOMAC | WOMAC |
|---|---|---|---|---|---|---|
| | | | Dose | average % NGF | (CFB) at | (CFP) at |
| Cohort | N | RoA | (μg/kg) | suppression | week 8 | week 8 |
| 8 | 12 | IV | 1 | 16 | 0.2 | 1.3 |
| 9 | 11 | IV | 5 | 18 | −0.8 | 0.4 |
| 10 | 9 | IV | 50 | 33 | −1.4 | −0.3 |
| 11 | 11 | IV | 150 | 37 | −2.4 | −1.2 |
| 12 | 7 | IV | 450 | 47 | −2.3 | −1.1 |

RoA Route of administration; change from baseline (CFB); change from placebo (CFP).

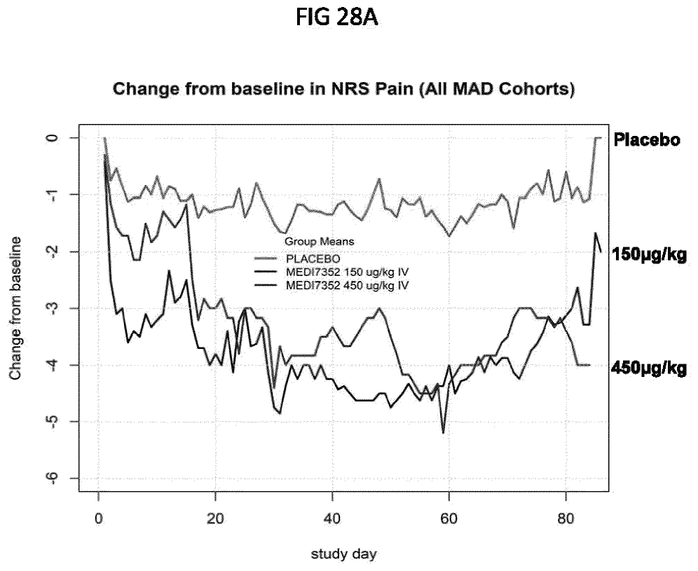

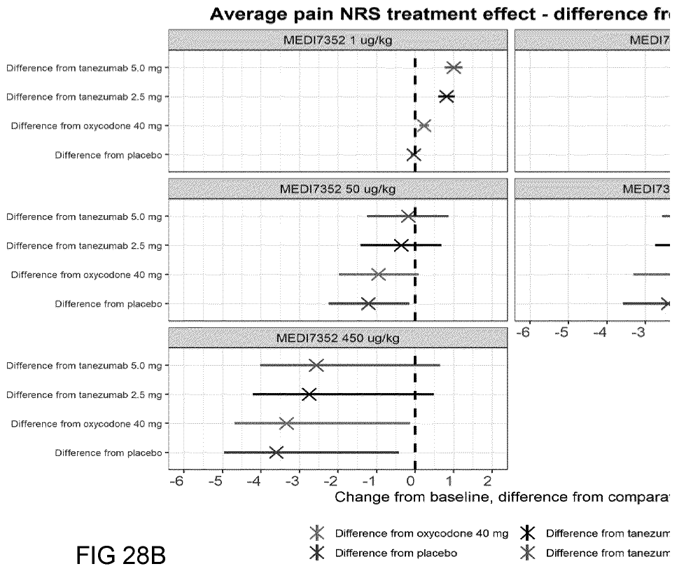

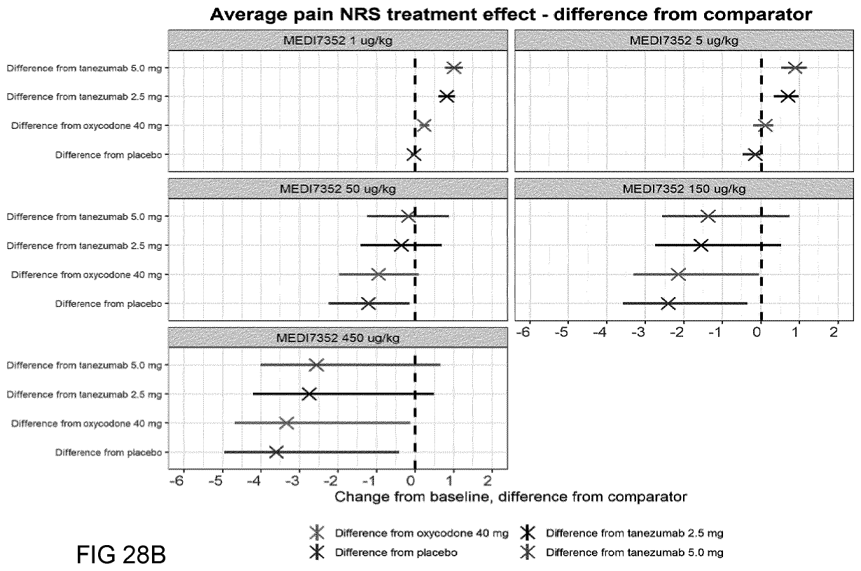

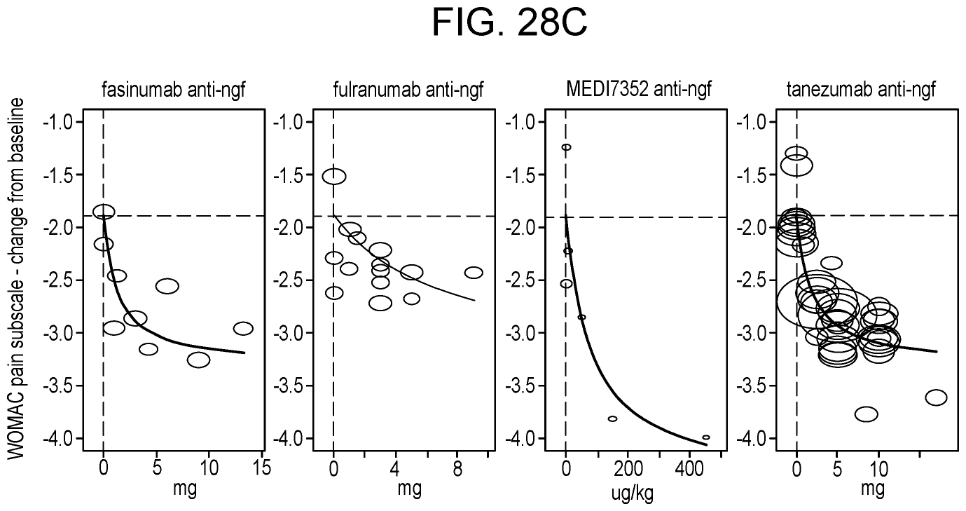

Following discharge, subjects were instructed to record pain daily on an 11-point NRS (0-10) at approximately the same time each morning, to reflect 24 hours of recall, to the end of the follow-up period. Repeated injections of either 150 μg/kg TNFR2-Fc_varB and 450 μg/kg TNFR2-Fc_varB clearly reduced pain compared to placebo-treated controls (FIG. 28A). Both of these doses also resulted in a larger reduction in pain compared to a 40 mg dose of the opioid oxycodone, a 2.5 mg dose of the anti-NGF antibody tanezumab or 5 mg tanezumab (FIG. 28B), and were more effective at reducing pain compared to the maximal pain reduction achieved with fasinumab, fulranumab or tanezumab (FIG. 28C).

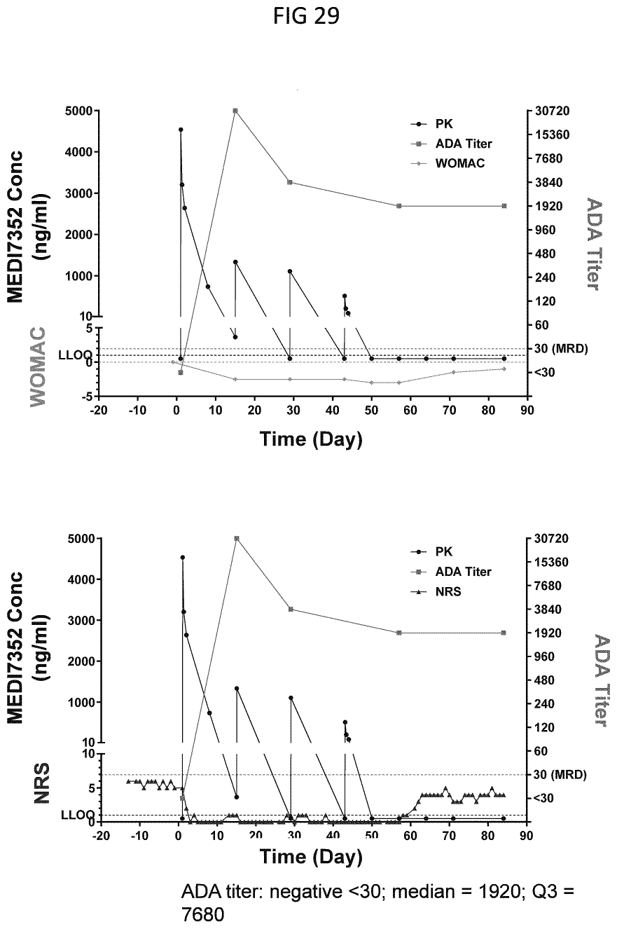

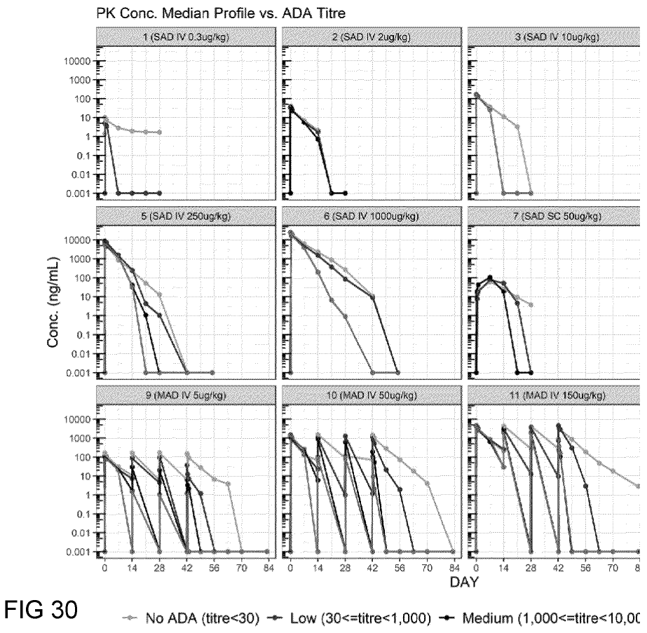

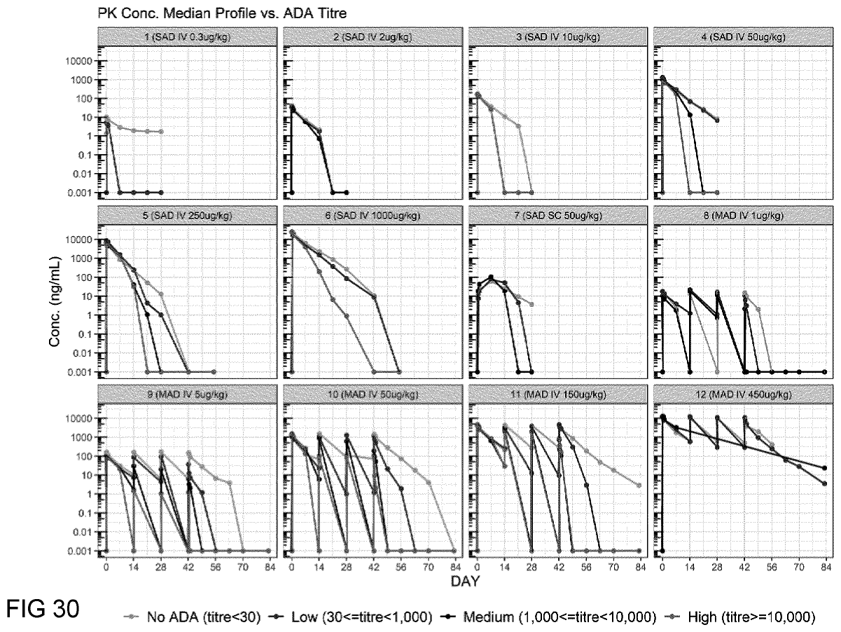

Anti-drug antibody (ADA) levels were measured by immunogenicity assessment (Food and Drug Administration. Guidance for industry Immunogenicity assessment for therapeutic protein products. August 2014. Available from: http://www.fda.gov/downloads/drugs/guidancecomplianceregulatoryinformation/guida nces/ucm338856.pdf. Accessed 27 Jul. 2018). Overall, ADA prevalence in subjects who received repeated doses of TNFR2-Fc_varB was 70% (35 of 50 subjects). ADA prevalence was defined as the proportion of subjects who were ADA positive at any time (baseline and/or post-baseline). There was no obvious relationship between TNFR2-Fc_varB dose levels and ADA prevalence, although a small portion of patients had a high ADA titer as shown in Table 5 below. To determine whether high ADA titer affects exposure and efficacy to TNFR2-Fc_varB, exposure to TNFR2-Fc_varB, ADA titer, and pain reduction were measured in an individual subject treated with 150 μg/kg TNFR2-Fc_varB. Surprisingly, despite the high ADA titer, significant exposure to TNFR2-Fc_varB was observed and pain reduction efficacy was maintained for >50 days (FIG. 29). In addition, the half-life of TNFR2-Fc-varB was higher in subjects who had lower ADA titers, and no patient treated with 450 μg/kg TNFR2-Fc_varB had a high ADA titer (FIG. 30). Importantly, there was no association between ADA and adverse events.

TABLE 5
Prevalence of ADA between dose groups
after preliminary analysis.

| | | % ADA + | Max titers | | | |
|---|---|---|---|---|---|---|
| | | (treated | (min-medium- | | 1000- | |
| Dose | | subjects) | max) | <1000 | 10,000 | >10,000 |
| 1 | μg/kg iv | 41.7% | 120-960- | 3 | 2 | 0 |
| | | (5/12) | 3840 | (60%) | (40%) | (0%) |
| 5 | μg/kg iv | 72.7% | 120-1920- | 3 | 4 | 1 |
| | | (8/11) | 15360 | (37.5%) | (50%) | (12.5%) |
| 50 | μg/kg iv | 88.9% | 480-3840- | 2 | 5 | 1 |
| | | (8/9) | 61440 | (25%) | (62.5%) | (12.5%) |
| 150 | μg/kg iv | 81.8% | 60-1920- | 2 | 5 | 2 |
| | | (9/11) | 30720 | (22.2%) | (55.6%) | (22.2%) |
| 450 | μg/kg iv | 71.4% | 30-480- | 4 | 1 | 0 |
| | | (5/7) | 1920 | (80%) | (20%) | (0%) |

| | | | | | |
|---|---|---|---|---|---|
| Overall | 70.0% | 30-1920- | 14 | 17 | 4 |
| | (35/50) | 61440 | (40%) | (48.6%) | (11.4%) |

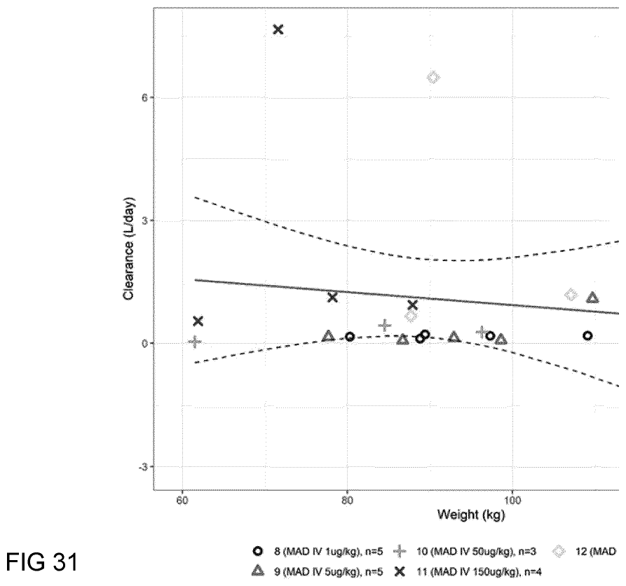

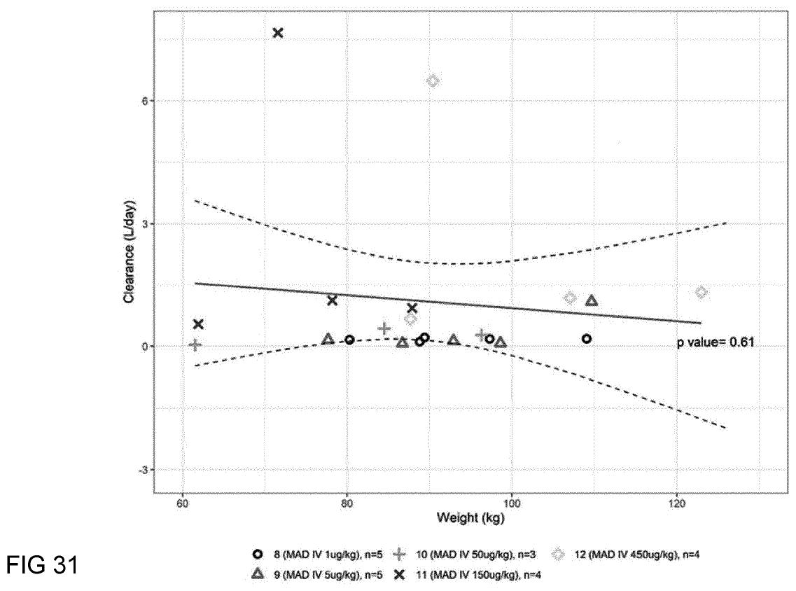

Surprisingly, using data from the MAD stage of the trial, the inventors also found that body weight is not a clinically significant covariate for exposure (p=0.61; FIG. 31).

### Example 10—Effects of Fixed Subcutaneous Doses of TNFR2-Fc_varB in Humans with Painful Osteoarthritis of the Knee

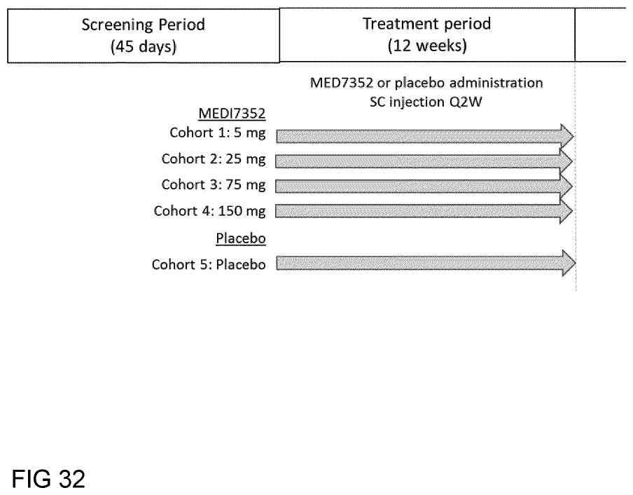

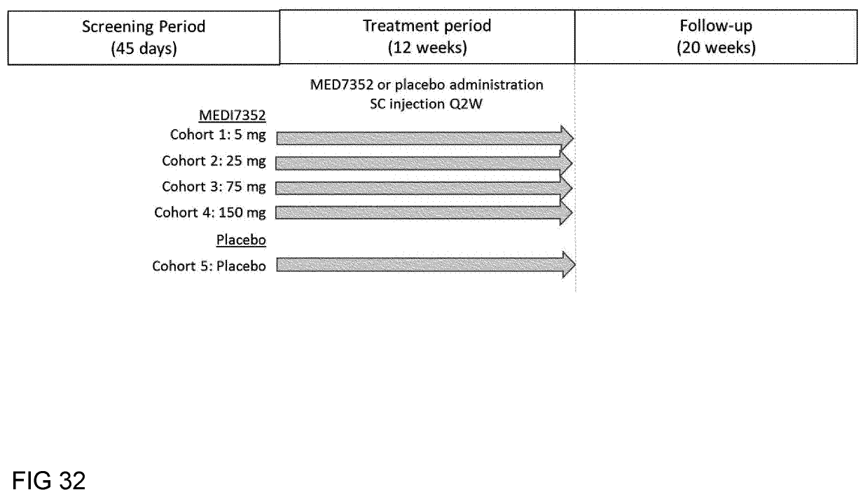

Based on the finding that body weight is not a clinically significant covariate for exposure to TNFR2-Fc_varB, the inventors hypothesized that a fixed dosing strategy of TNFR2-Fc_varB would be effective for the treatment of pain in humans. Thus, a multi-center, randomized, double-blind, placebo-controlled, clinical trial was designed for subjects 18 to 80 years of age with painful osteoarthritis of the knee. Approximately 300 eligible subjects will be randomly assigned to TNFR2-Fc_varB treatment or placebo to ensure that approximately 255 subjects complete the treatment period. Subjects will receive one of 4 fixed subcutaneous doses of TNFR2-Fc_varB (7.5 mg, 25 mg, 75 mg, and 150 mg) or placebo every 2 weeks (Q2W) over a 12-week period. These fixed subcutaneous doses of TNFR2-Fc_varB are predicted to provide similar effects to intravenous doses of 15, 50, 150, and 300 μg/kg TNFR2-Fc_varB respectively and were calculated based on the bioavailability observed for subcutaneously administered TNFR2-Fc_varB and the weight distribution of OA patients. In total, each subject will receive 6 doses of TNFR2-Fc_varB or placebo during the treatment period. A simplified layout of the study design is provided in FIG. 32.

Beginning 14 days prior to commencing treatment, subjects will be instructed to record pain daily on an 11-point NRS (0-10) at approximately the same time each morning, to reflect 24 hours of recall, until at least 6 weeks after the final administration of TNFR2-Fc_varB or placebo.

Subjects will be instructed to complete the WOMAC questionnaire at specific time points, beginning before commencement of treatment and ending at least 6 weeks after the final administration of TNFR2-Fc_varB or placebo

Subjects will be instructed to complete the Patient Global Assessment (PGA) at specific time points, beginning before commencement of treatment and ending at least 6 weeks after the final administration of TNFR2-Fc_varB or placebo. The PGA is a 5-point Likert scale used to assess symptoms and activity impairment due to OA of the knee. Subjects are asked to identify a number from 1=very good (asymptomatic and no limitation of normal activities) to 5=very poor (very severe symptoms which are intolerable and inability to carry out all normal activities) based on the question “Considering all the ways that OA of the knee affects you, how are you feeling today?”

The effect of TNFR2-Fc_varB on levels of free NGF in the periphery may be measured. For example, free NGF in the periphery may be measured weekly starting 1 day after administration up to week 12 and then measured again at week 18 and week 28.

The effect of TNFR2-Fc_varB on levels of total NGF in the periphery may be measured. For example, total NGF in the periphery may be measured weekly starting 1 day after administration up to week 12 and then measured again at week 18 and week 28.

As a proxy for measuring levels of TNFα levels, CXCL-13 levels may be measured. For example, CXCL-13 levels may be measured weekly starting 1 day after administration up to week 12 and then measured again at week 18 and week 28.

Sequence listing

SEQ ID NO: 1 NP_002497.2|beta-nerve growth factor precursor [Homo sapiens]

| | |
|---|---|
| 1 | MSMLFYTLIT AFLIGIQAEP HSESNVPAGH TIPQAHWTKL QHSLDTALRR ARSAPAAAIA |
| 61 | ARVAGQTRNI TVDPRLFKKR RLRSPRVLFS TQPPREAADT QDLDFEVGGA APFNRTHRSK |
| 121 | RSSSHPIFHR GEFSVCDSVS VWVGDKTTAT DIKGKEVMVL GEVNINNSVF KQYFFETKCR |
| 181 | DPNPVDSGCR GIDSKHWNSY CTTTHTFVKA LTMDGKQAAW RFIRIDTACV CVLSRKAVRR |
| 241 | A |

SEQ ID NO: 2 NP_000585.2|tumor necrosis factor [Homo sapiens]

| | |
|---|---|
| 1 | MSTESMIRDV ELAEEALPKK TGGPQGSRRC LFLSLFSFLI VAGATTLFCL LHFGVIGPQR |
| 61 | EEFPRDLSLI SPLAQAVRSS SRTPSDKPVA HVVANPQAEG QLQWLNRRAN ALLANGVELR |
| 121 | DNQLVVPSEG LYLIYSQVLF KGQGCPSTHV LLTHTISRIA VSYQTKVNLL SAIKSPCQRE |
| 181 | TPEGAEAKPW YEPIYLGGVF QLEKGDRLSA EINRPDYLDF AESGQVYFGI IAL |

SEQ ID NO: 3 MEDI-578 VH (1256A5 VH)

| | |
|---|---|
| 1 | QVQLVQSGAE VKKPGSSVKV SCKASGGTFS TYGISWVRQA PGQGLEWMGG IIPIFDTGNS |
| 61 | AQSFQGRVTI TADESTSTAY MELSSLRSED TAVYYCARSS RIYDLNPSLT AYYDMDVWGQ |
| 121 | GTMVTVSS |

SEQ ID NO: 4 MEDI-578 VHCDR1

| | |
|---|---|
| 1 | TYGIS |

SEQ ID NO: 5 MEDI-578 VHCDR2

| | |
|---|---|
| 1 | GIIPIFDTGN SAQSFQG |

SEQ ID NO: 6 MEDI-578 VHCDR3

| | |
|---|---|
| 1 | SSRIYDLNPS LTAYYDMDV |

SEQ ID NO: 7 MEDI-578 VL (1256A5 VL)

| | |
|---|---|
| 1 | QSVLTQPPSV SAAPGQKVTI SCSGSSSNIG NNYVSWYQQL PGTAPKLLIY DNNKRPSGIP |
| 61 | DRFSGSKSGT SATLGITGLQ TGDEADYYCG TWDSSLSAWV FGGGTKLTVL |

SEQ ID NO: 8 MEDI-578 VLCDR1

| | |
|---|---|
| 1 | SGSSSNIGNN YVS |

SEQ ID NO: 9 MEDI-578 VLCDR2

| | |
|---|---|
| 1 | DNNKRPS |

SEQ ID NO: 10 MEDI-578 VLCDR3

| | |
|---|---|
| 1 | GTWDSSLSAW V |

SEQ ID NO: 11

| | |
|---|---|
| 1 | SSRIYDENSA LISYYDMDV |

SEQ ID NO: 12

| | |
|---|---|
| 1 | SSRIYDMISS LQPYYDMDV |

SEQ ID NO: 13 soluble TNFR2 amino acid sequence

| | |
|---|---|
| 1 | LPAQVAFTPY APEPGSTCRL REYYDQTAQM CCSKCSPGQH AKVFCTKTSD TVCDSCEDST |
| 61 | YTQLWNWVPE CLSCGSRCSS DQVETQACTR EQNRICTCRP GWYCALSKQE GCRLCAPLRK |
| 121 | CRPGFGVARP GTETSDVVCK PCAPGTFSNT TSSTDICRPH QICNVVAIPG NASMDAVCTS |
| 181 | TSPTRSMAPG AVHLPQPVST RSQHTQPTPE PSTAPSTSFL LPMGPSPPAE GSTGDEPKSC |
| 241 | DKTHTCPPCP APELLGGPSV FLFPPKPKDT LMISRTPEVT CVVVDVSHED PEVKENWYVD |
| 301 | GVEVHNAKTK PREEQYNSTY RVVSVLTVLH QDWLNGKEYK CKVSNKALPA PIEKTISKAK |
| 361 | GQPREPQVYT LPPSREEMTK NQVSLTCLVK GFYPSDIAVE WESNGQPENN YKTTPPVLDS |
| 421 | DGSFFLYSKL TVDKSRWQQG NVFSCSVMHE ALHNHYTQKS LSLSPGK |

SEQ ID NO: 14 TNFR2-Fc_VH#4-amino acid sequence

| | |
|---|---|
| 1 | LPAQVAFTPY APEPGSTCRL REYYDQTAQM CCSKCSPGQH AKVFCTKTSD TVCDSCEDST |
| 61 | YTQLWNWVPE CLSCGSRCSS DQVETQACTR EQNRICTCRP GWYCALSKQE GCRLCAPLRK |
| 121 | CRPGFGVARP GTETSDVVCK PCAPGTFSNT TSSTDICRPH QICNVVAIPG NASMDAVCTS |
| 181 | TSPTRSMAPG AVHLPQPVST RSQHTQPTPE PSTAPSTSFL LPMGPSPPAE GSTGDEPKSC |
| 241 | DKTHTCPPCP APELLGGPSV FLFPPKPKDT LMISRTPEVT CVVVDVSHED PEVKENWYVD |
| 301 | GVEVHNAKTK PREEQYNSTY RVVSVLTVLH QDWLNGKEYK CKVSNKALPA PIEKTISKAK |
| 361 | GQPREPQVYT LPPSREEMTK NQVSLTCLVK GFYPSDIAVE WESNGQPENN YKTTPPVLDS |
| 421 | DGSFFLYSKL TVDKSRWQQG NVFSCSVMHE ALHNHYTQKS LSLSPGKGGG GSGGGGSQVQ |
| 481 | LVQSGAEVKK PGSSVKVSCK ASGGTFSTYG ISWVRQAPGQ GLEWMGGIIP IFDTGNSAQS |
| 541 | FQGRVTITAD ESTSTAYMEL SSLRSEDTAV YYCARSSRIY DLNPSLTAYY DMDVWGQGTM |
| 601 | VTVSSGGGGS GGGGSGGGGS AQSVLTQPPS VSAAPGQKVT ISCSGSSSNI GNNYVSWYQQ |
| 661 | LPGTAPKLLI YDNNKRPSGI PDRFSGSKSG TSATLGITGL QTGDEADYYC GTWDSSLSAW |
| 721 | VFGGGTKLTV L |

SEQ ID NO: 15 (Gly₄Ser)₃ 15 aa linker sequence

| | |
|---|---|
| 1 | GGGGSGGGGS GGGGS |

SEQ ID NO: 16 TNFR2-Fc_VH#4-nucleotide sequence

| | |
|---|---|
| 1 | CTGCCCGCCC AGGTGGCCTT TACCCCTTAT GCCCCCGAGC CCGGCAGCAC CTGTCGGCTG |
| 61 | AGAGAGTACT ACGACCAGAC CGCCCAGATG TGCTGCAGCA AGTGCTCTCC TGGCCAGCAT |
| 121 | GCCAAGGTGT TCTGCACCAA GACCAGCGAC ACCGTGTGCG ACAGCTGCGA GGACAGCACC |
| 181 | TACACCCAGC TGTGGAACTG GGTGCCCGAG TGCCTGAGCT GCGGCAGCAG ATGCAGCAGC |
| 241 | GACCAGGTGG AAACCCAGGC CTGCACCAGA GAGCAGAACC GGATCTGCAC CTGTAGACCC |
| 301 | GGCTGGTACT GCGCCCTGAG CAAGCAGGAA GGCTGCAGAC TCTGCGCCCC TCTGCGGAAG |
| 361 | TGCAGACCCG GCTTTGGCGT GGCCAGACCC GGCACCGAGA CAAGCGACGT GGTCTGTAAG |
| 421 | CCCTGCGCTC CTGGCACCTT CAGCAACACC ACCAGCAGCA CCGACATCTG CAGACCCCAC |
| 481 | CAGATCTGCA ACGTGGTGGC CATCCCCGGC AACGCCAGCA TGGATGCCGT CTGCACCAGC |
| 541 | ACTAGCCCCA CCAGAAGTAT GGCCCCTGGC GCCGTGCATC TGCCCCAGCC TGTGTCCACC |
| 601 | AGAAGCCAGC ACACCCAGCC CACCCCTGAG CCTAGCACCG CCCCCTCCAC CAGCTTTCTG |
| 661 | CTGCCTATGG GCCCTAGCCC TCCAGCCGAG GGAAGCACAG GCGACGAGCC CAAGAGCTGC |
| 721 | GACAAGACCC ACACCTGTCC CCCCTGCCCT GCCCCTGAAC TGCTGGGCGG ACCCAGCGTG |
| 781 | TTCCTGTTCC CCCCAAAGCC CAAGGACACC CTGATGATCA GCCGGACCCC CGAAGTGACC |
| 841 | TGCGTGGTGG TGGACGTGTC CCACGAGGAC CCTGAAGTGA AGTTCAATTG GTACGTGGAC |
| 901 | GGCGTGGAAG TGCACAACGC CAAGACCAAG CCCAGAGAGG AACAGTACAA CTCCACCTAC |
| 961 | CGGGTGGTGT CCGTGCTGAC CGTGCTGCAC CAGGACTGGC TGAACGGCAA AGAGTACAAG |
| 1021 | TGCAAGGTCT CCAACAAGGC CCTGCCTGCC CCCATCGAGA AAACCATCAG CAAGGCCAAG |
| 1081 | GGCCAGCCCC GCGAGCCTCA GGTGTACACA CTGCCCCCCA GCCGGGAAGA GATGACCAAG |
| 1141 | AACCAGGTGT CCCTGACCTG CCTGGTCAAA GGCTTCTACC CCAGCGATAT CGCCGTGGAA |
| 1201 | TGGGAGAGCA ATGGCCAGCC CGAGAACAAC TACAAGACCA CCCCCCCTGT GCTGGACAGC |
| 1261 | GACGGCTCAT TCTTCCTGTA CAGCAAGCTG ACCGTGGACA AGAGCCGGTG GCAGCAGGGC |
| 1321 | AACGTGTTCA GCTGCAGCGT GATGCACGAG GCCCTGCACA ACCACTACAC CCAGAAGTCC |
| 1381 | CTGAGCCTGA GCCCCGGAAA GGGCGGTGGC GGATCCGGAG GTGGGGGATC TCAGGTGCAG |
| 1441 | CTGGTGCAGT CTGGCGCCGA AGTGAAGAAA CCCGGCTCTA GCGTGAAGGT GTCCTGCAAG |
| 1501 | GCCAGCGGCG GCACCTTCTC CACCTACGGC ATCAGCTGGG TCCGCCAGGC CCCTGGACAG |
| 1561 | GGCCTGGAAT GGATGGGCGG CATCATCCCC ATCTTCGACA CCGGCAACAG CGCCCAGAGC |
| 1621 | TTCCAGGGCA GAGTGACCAT CACCGCCGAC GAGAGCACCT CCACCGCCTA CATGGAACTG |
| 1681 | AGCAGCCTGC GGAGCGAGGA CACCGCCGTG TACTACTGCG CCAGAAGCAG CCGGATCTAC |
| 1741 | GACCTGAACC CCAGCCTGAC CGCCTACTAC GACATGGACG TGTGGGGCCA GGGCACCATG |
| 1801 | GTCACAGTGT CTAGCGGAGG CGGCGGATCT GGCGGCGGAG GAAGTGGCGG GGGAGGATCT |
| 1861 | GCCCAGAGCG TGCTGACCCA GCCCCCTTCT GTGTCTGCCG CCCCTGGCCA GAAAGTGACC |
| 1921 | ATCTCCTGCA GCGGCAGCAG CAGCAACATC GGCAACAACT ACGTGTCCTG GTATCAGCAG |
| 1981 | CTGCCCGGCA CCGCCCCTAA GCTGCTGATC TACGACAACA ACAAGCGGCC CAGCGGCATC |
| 2041 | CCCGACCGGT TTAGCGGCAG CAAGAGCGGG ACTTCTGCTA CACTGGGCAT CACAGGCCTG |
| 2101 | CAGACCGGCG ACGAGGCCGA CTACTACTGC GGCACCTGGG ACAGCAGCCT GAGCGCTTGG |
| 2161 | GTGTTCGGCG GAGGCACCAA GCTGACAGTG CTG |

SEQ ID NO: 17-TNFR2-Fc_varB-amino acid sequence

| | |
|---|---|
| 1 | LPAQVAFTPY APEPGSTCRL REYYDQTAQM CCSKCSPGQH AKVFCTKTSD TVCDSCEDST |
| 61 | YTQLWNWVPE CLSCGSRCSS DQVETQACTR EQNRICTCRP GWYCALSKQE GCRLCAPLRK |
| 121 | CRPGFGVARP GTETSDVVCK PCAPGTFSNT TSSTDICRPH QICNVVAIPG NASMDAVCTS |
| 181 | TSPTRSMAPG AVHLPQPVST RSQHTQPTPE PSTAPSTSFL LPMGPSPPAE GSTGDEPKSC |
| 241 | DKTHTCPPCP APELLGGPSV FLFPPKPKDT LMISRTPEVT CVVVDVSHED PEVKFNWYVD |
| 301 | GVEVHNAKTK PREEQYNSTY RVVSVLTVLH QDWLNGKEYK CKVSNKALPA PIEKTISKAK |
| 361 | GQPREPQVYT LPPSREEMTK NQVSLTCLVK GFYPSDIAVE WESNGQPENN YKTTPPVLDS |
| 421 | DGSFFLYSKL TVDKSRWQQG NVFSCSVMHE ALHNHYTQKS LSLSPGKGGG GSGGGGSQVQ |
| 481 | LVQSGAEVKK PGSSVKVSCK ASGGTFSTYG ISWVRQAPGQ CLEWMGGIIP IFDTGNSAQS |
| 541 | FQGRVTITAD ESTSTAYMEL SSLRSEDTAV YYCARSSRIY DLNPSLTAYY DMDVWGQGTM |
| 601 | VTVSSGGGGS GGGGSGGGGS GGGGSQSVLT QPPSVSAAPG QKVTISCSGS SSNIGNNYVS |
| 661 | WYQQLPGTAP KLLIYDNNKR PSGIPDRFSG SKSGTSATLG ITGLQTGDEA DYYCGTWDSS |
| 721 | LSAWVFGCGT KLTVL |

SEQ ID NO: 18-TNFR2-Fc_varB-nucleotide sequence

| | |
|---|---|
| 1 | CTGCCCGCCC AGGTGGCCTT TACCCCTTAT GCCCCCGAGC CCGGCAGCAC CTGTCGGCTG |
| 61 | AGAGAGTACT ACGACCAGAC CGCCCAGATG TGCTGCAGCA AGTGCTCTCC TGGCCAGCAT |
| 121 | GCCAAGGTGT TCTGCACCAA GACCAGCGAC ACCGTGTGCG ACAGCTGCGA GGACAGCACC |
| 181 | TACACCCAGC TGTGGAACTG GGTGCCCGAG TGCCTGAGCT GCGGCAGCAG ATGCAGCAGC |
| 241 | GACCAGGTGG AAACCCAGGC CTGCACCAGA GAGCAGAACC GGATCTGCAC CTGTAGACCC |
| 301 | GGCTGGTACT GCGCCCTGAG CAAGCAGGAA GGCTGCAGAC TCTGCGCCCC TCTGCGGAAG |
| 361 | TGCAGACCCG GCTTTGGCGT GGCCAGACCC GGCACCGAGA CAAGCGACGT GGTCTGCAAG |
| 421 | CCCTGCGCTC CTGGCACCTT CAGCAACACC ACCAGCAGCA CCGACATCTG CAGACCCCAC |
| 481 | CAGATCTGCA ACGTGGTGGC CATCCCCGGC AACGCCAGCA TGGATGCCGT GTGCACCAGC |
| 541 | ACCAGCCCCA CCAGAAGTAT GGCCCCTGGC GCCGTGCATC TGCCCCAGCC TGTGTCCACC |
| 601 | AGAAGCCAGC ACACCCAGCC CACCCCTGAG CCTAGCACCG CCCCCTCCAC CAGCTTTCTG |
| 661 | CTGCCTATGG GCCCTAGCCC TCCAGCCGAG GGAAGCACAG GCGACGAGCC CAAGAGCTGC |
| 721 | GACAAGACCC ACACCTGTCC CCCCTGCCCT GCCCCTGAAC TGCTGGGCGG ACCCAGCGTG |
| 781 | TTCCTGTTCC CCCCAAAGCC CAAGGACACC CTGATGATCA GCCGGACCCC CGAAGTGACC |
| 841 | TGCGTGGTGG TGGACGTGTC CCACGAGGAC CCTGAAGTGA AGTTCAATTG GTACGTGGAC |
| 901 | GGCGTGGAAG TGCACAACGC CAAGACCAAG CCCAGAGAGG AACAGTACAA CTCCACCTAC |
| 961 | CGGGTGGTGT CCGTGCTGAC CGTGCTGCAC CAGGACTGGC TGAACGGCAA AGAGTACAAG |
| 1021 | TGCAAAGTCT CCAACAAGGC CCTGCCTGCC CCCATCGAGA AAACCATCAG CAAGGCCAAG |
| 1081 | GGCCAGCCCC GCGAGCCTCA gGTGTACACA CTGCCCCCCA GCCGGGAAGA GATGACCAAG |
| 1141 | AACCAGGTGT CCCTGACCTG CCTGGTCAAA GGCTTCTACC CCAGCGATAT CGCCGTGGAA |
| 1201 | TGGGAGAGCA ACGGCCAGCC CGAGAACAAC TACAAGACCA CCCCCCCTGT GCTGGACAGC |
| 1261 | GACGGCTCAT TCTTCCTGTA CAGCAAGCTG ACCGTGGACA AGAGCCGGTG GCAGCAGGGC |
| 1321 | AATGTCTTCA GCTGTAGCGT GATGCACGAG GCCCTGCACA ACCACTACAC CCAGAAGTCC |
| 1381 | CTGAGCCTGA GCCCCGGAAA GGGCGGAGGC GGATCCGGAG GTGGGGGATC TCAGGTGCAG |
| 1441 | CTGGTGCAGT CTGGCGCCGA AGTGAAGAAA CCCGGCTCTA GCGTGAAGGT GTCCTGCAAG |
| 1501 | GCCAGCGGCG GCACCTTCTC CACCTACGGC ATCAGCTGGG TCCGCCAGGC CCCTGGACAG |
| 1561 | TGTCTGGAAT GGATGGGCGG CATCATCCCC ATCTTCGACA CCGGCAACAG CGCCCAGAGC |
| 1621 | TTCCAGGGCA GAGTGACCAT CACCGCCGAC GAGAGCACCT CCACCGCCTA CATGGAACTG |
| 1681 | AGCAGCCTGC GGAGCGAGGA CACCGCCGTG TACTACTGCG CCAGAAGCAG CCGGATCTAC |
| 1741 | GACCTGAACC CCAGCCTGAC CGCCTACTAC GACATGGACG TGTGGGGCCA GGGCACCATG |
| 1801 | GTCACAGTGT CTAGCGGAGG CGGAGGCAGC GGAGGTGGTG GATCTGGTGG CGGAGGAAGT |
| 1861 | GGCGGCGGAG GCTCTCAGAG CGTGCTGACC CAGCCCCCTT CTGTGTCTGC CGCCCCTGGC |
| 1921 | CAGAAAGTGA CCATCTCCTG CAGCGGCAGC AGCAGCAACA TCGGCAACAA CTACGTGTCC |
| 1981 | TGGTATCAGC AGCTGCCCGG CACCGCCCCT AAGCTGCTGA TCTACGACAA CAACAAGCGG |
| 2041 | CCCAGCGGCA TCCCCGACCG GTTTAGCGGC AGCAAGAGCG GGACTTCTGC TACACTGGGC |
| 2101 | ATCACAGGCC TGCAGACCGG CGACGAGGCC GACTACTACT GCGGCACCTG GGACAGCAGC |
| 2161 | CTGAGCGCTT GGGTGTTCGG CTGCGGCACC AAGCTGACAG TGCTG |

SEQ ID NO: 19-(Gly₄Ser)₄ 20 aa linker sequence

| | |
|---|---|
| 1 | GGGGSGGGGS GGGGGGGGS |

SEQ ID NO: 20-ndimab varB-L chain amino acid sequence

| | |
|---|---|
| 1 | EIVLTQSPAT LSLSPGERAT LSCRASQSVY SYLAWYQQKP GQAPRLLIYD ASNRAIGIPA |
| 61 | RFSGSGSGTD FTLTISSLEP EDFAVYYCQQ RSNWPPFTFG PGTKVDIKRT VAAPSVFIFP |
| 121 | PSDEQLKSGT ASVVCLLNNF YPREAKVQWK VDNALQSGNS QESVTEQDSK DSTYSLSSTL |
| 181 | TLSKADYEKH KVYACEVTHQ GLSSPVTKSF NRGEC |

SEQ ID NO: 21-ndimab varB-L chain nucleotide sequence

| | |
|---|---|
| 1 | GAAATCGTGC TGACCCAGAG CCCCGCCACC CTGTCTCTGA GCCCTGGCGA GAGAGCCACC |
| 61 | CTGAGCTGCA GAGCCAGCCA GAGCGTGTAC TCCTACCTGG CTTGGTATCA GCAGAAGCCC |
| 121 | GGCCAGGCCC CCAGACTGCT GATCTACGAC GCCAGCAACC GGGCCATCGG CATCCCTGCC |
| 181 | AGATTTTCTG GCAGCGGCAG CGGCACCGAC TTCACCCTGA CCATCAGCAG CCTGGAACCC |
| 241 | GAGGACTTCG CCGTGTACTA CTGCCAGCAG CGGAGCAACT GGCCCCCCTT CACCTTCGGC |
| 301 | CCTGGCACCA AGGTGGACAT CAAGCGTACG GTGGCTGCAC CATCTGTCTT CATCTTCCCG |
| 361 | CCATCTGATG AGCAGTTGAA ATCTGGAACT GCCTCTGTTG TGTGCCTGCT GAATAACTTC |
| 421 | TATCCCAGAG AGGCCAAAGT ACAGTGGAAG GTGGATAACG CCCTCCAATC GGGTAACTCC |
| 481 | CAGGAGAGTG TCACAGAGCA GGACAGCAAG GACAGCACCT ACAGCCTCAG CAGCACCCTG |
| 541 | ACGCTGAGCA AAGCAGACTA CGAGAAACAC AAAGTCTACG CCTGCGAAGT CACCCATCAG |
| 601 | GGCCTGAGCT CGCCCGTCAC AAAGAGCTTC AACAGGGGAG AGTGT |

SEQ ID NO: 22-ndimab varB-H chain amino acid sequence

| | |
|---|---|
| 1 | QVQLVESGGG VVQPGRSLRL SCAASGFIFS SYAMHWVRQA PGNGLEWVAF MSYDGSNKKY |
| 61 | ADSVKGRFTI SRDNSKNTLY LQMNSLRAED TAVYYCARDR GISAGGNYYY YGMDVWGQGT |
| 121 | TVTVSSASTK GPSVFPLAPS SKSTSGGTAA LGCLVKDYFP EPVTVSWNSG ALTSGVHTFP |
| 181 | AVLQSSGLYS LSSVVTVPSS SLGTQTYICN VNHKPSNTKV DKRVEPKSCD KTHTCPPCPA |
| 241 | PELLGGPSVF LFPPKPKDTL MISRTPEVTC VVVDVSHEDP EVKENWYVDG VEVHNAKTKP |
| 301 | REEQYNSTYR VVSVLTVLHQ DWLNGKEYKC KVSNKALPAP IEKTISKAKG QPREPQVYTL |
| 361 | PPSREEMTKN QVSLTCLVKG FYPSDIAVEW ESNGQPENNY KTTPPVLDSD GSFFLYSKLT |
| 421 | VDKSRWQQGN VFSCSVMHEA LHNHYTQKSL SLSPGKGGGG SGGGGSQVQL VQSGAEVKKP |
| 481 | GSSVKVSCKA SGGTFSTYGI SWVRQAPGQC LEWMGGIIPI FDTGNSAQSF QGRVTITADE |
| 541 | STSTAYMELS SLRSEDTAVY YCARSSRIYD LNPSLTAYYD MDVWGQGTMV TVSSGGGGSG |
| 601 | GGGSGGGGSG GGGSQSVLTQ PPSVSAAPGQ KVTISCSGSS SNIGNNYVSW YQQLPGTAPK |
| 661 | LLIYDNNKRP SGIPDRFSGS KSGTSATLGI TGLQTGDEAD YYCGTWDSSL SAWVFGCGTK |
| 721 | LTVL |

SEQ ID NO: 23-ndimab varB-H chain nucleotide sequence

| | |
|---|---|
| 1 | CAGGTGCAGC TGGTGGAAAG CGGCGGAGGC GTGGTGCAGC CCGGCAGAAG CCTGAGACTG |
| 61 | AGCTGCGCTG CCAGCGGCTT CATCTTCAGC AGCTACGCCA TGCACTGGGT CCGCCAGGCC |
| 121 | CCTGGCAACG GACTGGAATG GGTGGCCTTC ATGAGCTACG ACGGCAGCAA CAAGAAGTAC |
| 181 | GCCGACAGCG TGAAGGGCCG GTTCACCATC AGCCGGGACA ACAGCAAGAA CACCCTGTAC |
| 241 | CTGCAGATGA ACAGCCTGCG GGCTGAGGAC ACCGCCGTGT ACTACTGCGC CAGAGACCGA |
| 301 | GGCATCAGTG CTGGCGGCAA CTACTACTAC TACGGCATGG ACGTGTGGGG CCAGGGCACC |
| 361 | ACCGTGACCG TGTCTAGCGC GTCGACCAAG GGCCCATCCG TCTTCCCCCT GGCACCCTCC |
| 421 | TCCAAGAGCA CCTCTGGGGG CACAGCGGCC CTGGGCTGCC TGGTCAAGGA CTACTTCCCC |
| 481 | GAACCGGTGA CGGTGTCCTG GAACTCAGGC GCTCTGACCA GCGGCGTGCA CACCTTCCCG |
| 541 | GCTGTCCTAC AGTCCTCAGG ACTCTACTCC CTCAGCAGCG TGGTGACCGT GCCCTCCAGC |
| 601 | AGCTTGGGCA CCCAGACCTA CATCTGCAAC GTGAATCACA AGCCCAGCAA CACCAAGGTG |
| 661 | GACAAGAGAG TTGAGCCCAA ATCTTGTGAC AAAACTCACA CATGCCCACC GTGCCCAGCA |
| 721 | CCTGAACTCC TGGGGGGACC GTCAGTCTTC CTCTTCCCCC CAAAACCCAA GGACACCCTC |
| 781 | ATGATCTCCC GGACCCCTGA GGTCACATGC GTGGTGGTGG ACGTGAGCCA CGAAGACCCT |
| 841 | GAGGTCAAGT TCAACTGGTA CGTGGACGGC GTGGAGGTGC ATAATGCCAA GACAAAGCCG |
| 901 | CGGGAGGAGC AGTACAACAG CACGTACCGT GTGGTCAGCG TCCTCACCGT CCTGCACCAG |
| 961 | GACTGGCTGA ATGGCAAGGA GTACAAGTGC AAGGTCTCCA ACAAAGCCCT CCCAGCCCCC |
| 1021 | ATCGAGAAAA CCATCTCCAA AGCCAAAGGG CAGCCCCGAG AACCACAGGT CTACACCCTG |
| 1081 | CCCCCATCCC GGGAGGAGAT GACCAAGAAC CAGGTCAGCC TGACCTGCCT GGTCAAAGGC |
| 1141 | TTCTATCCCA GCGACATCGC CGTGGAGTGG GAGAGCAATG GGCAGCCGGA GAACAACTAC |
| 1201 | AAGACCACGC CTCCCGTGCT GGACTCCGAC GGCTCCTTCT TCCTCTATAG CAAGCTCACC |
| 1261 | GTGGACAAGA GCAGGTGGCA GCAGGGGAAC GTCTTCTCAT GCTCCGTGAT GCATGAGGCT |
| 1321 | CTGCACAACC ACTACACGCA GAAGAGCCTC TCCCTGTCTC CGGGTAAAGG CGGAGGGGGA |
| 1381 | TCCGGCGGAG GGGGCTCTCA GGTGCAGCTG GTGCAGTCTG GCGCCGAAGT GAAGAAACCC |
| 1441 | GGCTCTAGCG TGAAGGTGTC CTGCAAGGCC AGCGGCGGCA CCTTCTCCAC CTACGGCATC |
| 1501 | AGCTGGGTCC GCCAGGCCCC TGGACAGTGT CTGGAATGGA TGGGCGGCAT CATCCCCATC |
| 1561 | TTCGACACCG GCAACAGCGC CCAGAGCTTC CAGGGCAGAG TGACCATCAC CGCCGACGAG |
| 1621 | AGCACCTCCA CCGCCTACAT GGAACTGAGC AGCCTGCGGA GCGAGGACAC CGCCGTGTAC |
| 1681 | TACTGCGCCA GAAGCAGCCG GATCTACGAC CTGAACCCCA GCCTGACCGC CTACTACGAC |
| 1741 | ATGGACGTGT GGGGCCAGGG CACCATGGTC ACAGTGTCTA GCGGAGGCGG AGGCAGCGGA |
| 1801 | GGTGGTGGAT CTGGTGGCGG AGGAAGTGGC GGCGGAGGCT CTCAGAGCGT GCTGACCCAG |
| 1861 | CCCCCTTCTG TGTCTGCCGC CCCTGGCCAG AAAGTGACCA TCTCCTGCAG CGGCAGCAGC |
| 1921 | AGCAACATCG GCAACAACTA CGTGTCCTGG TATCAGCAGC TGCCCGGCAC CGCCCCTAAG |
| 1981 | CTGCTGATCT ACGACAACAA CAAGCGGCCC AGCGGCATCC CCGACCGGTT TAGCGGCAGC |
| 2041 | AAGAGCGGGA CTTCTGCTAC ACTGGGCATC ACAGGCCTGC AGACCGGCGA CGAGGCCGAC |
| 2101 | TACTACTGCG GCACCTGGGA CAGCAGCCTG AGCGCTTGGG TGTTCGGCTG CGGCACCAAG |
| 2161 | CTGACAGTGC TG |

SEQ ID NO: 24-NGF-NG VH amino acid sequence
QVQLVQSGAEVKKPGSSVKVSCKASGGTFWFGAFTWVRQAPGQGLEWMGGIIPIFGLTNLAQNFQGRVTITADES
TSTVYMELSSLRSEDTAVYYCARSSRIYDLNPSLTAYYDMDVWGQGTMVTVSS
SEQ ID NO: 25-NGF-NG VH nucleotide sequence

| | |
|---|---|
| caggtgcagc tggtgcagtc tggggctgag gtgaagaagc ctgggtcctc ggtgaaggtc | 60 |
| tcctgcaagg cctctggagg caccttctgg ttcggcgcgt tcacctgggt gcgacaggcc | 120 |
| cctggacaag gacttgagtg gatgggaggg attattccta tcttcgggtt gacgaacttg | 180 |
| gcacagaact tccagggcag agtcacgatt accgcggacg aatccacgag cacagtctac | 240 |
| atggagctga gcagcttgag atctgaagac acggccgtat attattgtgc acgttcaagt | 300 |
| cgtatctacg atctgaaccc gtccctgacc gcctactacg atatggatgt ctggggccag | 360 |
| gggacaatgg tcaccgtctc gagt | 384 |

SEQ ID NO: 26-NGF-NG VL amino acid sequence
QSVLTQPPSVSAAPGQKVTISCSGSSSDIGNNYVSWYQQLPGTAPKLLIYDNNKRPSGIPDRFSGSKSGTSATLG
ITGLQTGDEADYYCGTWDSSLSAWVFGGGTKLTVL
SEQ ID NO: 27-NGF-NG VL nucleotide sequence

| | |
|---|---|
| cagtctgtgc tgactcagcc gccatcagtg tctgcggccc caggacagaa ggtcaccatc | 60 |
| tcctgctctg gaagcagctc cgacattggg aataattatg tatcgtggta ccagcagctc | 120 |
| ccaggaacag cccccaaact cctcatttat gacaataata agcgaccctc agggattcct | 180 |
| gaccgattct ctggctccaa gtctggcacg tcagccaccc tgggcatcac cggactccag | 240 |
| actggggacg aggccgatta ttactgcgga acatgggata gcagcctgag tgcttgggtg | 300 |
| ttcggcggag ggaccaagct gaccgtccta | 330 |

SEQ ID NO: 28-ndimab VH amino acid sequence

| | |
|---|---|
| 1 | QVQLVESGGG VVQPGRSLRL SCAASGFIFS SYAMHWVRQA PGNGLEWVAF MSYDGSNKKY |
| 61 | ADSVKGRFTI SRDNSKNTLY LQMNSLRAED TAVYYCARDR GISAGGNYYY YGMDVWGQGT |
| 121 | TVTVSS |

SEQ ID NO: 29-ndimab VL amino acid sequence

| | |
|---|---|
| 1 | EIVLTQSPAT LSLSPGERAT LSCRASQSVY SYLAWYQQKP GQAPRLLIYD ASNRAIGIPA |
| 61 | RFSGSGSGTD FTLTISSLEP EDFAVYYCQQ RSNWPPFTFG PGTKVDIK |

SEQ ID NO: 30-1126F1 VH amino acid sequence
EVQLVQTGAEVKKPGSSVKVSCKASGGTESTYGISWVRQAPGQGLEWIGGIIPIFDTGNSAQSFQGRVTITADES
TSTAYMEVSSLRSDDTAVYYCASSSRIYDANRQAVPYYDMDVWGQGTMVTVSS
SEQ ID NO: 31-1126F1 VL amino acid sequence
QAVLTQPSSVSTPPGQMVTISCSGSSSDIGNNYVSWYQQLPGTAPKLLIYDNNKRPSGIPDRFSGSKSGTSATLG
ITGLQTGDEADYYCGTWDSSLSAWVFGGGTKLTVL
SEQ ID NO: 32-1126G5 VH amino acid sequence
EVQLVQSGAEVKKPGSSVKVSCKASGGTESTYGISWVRQAPGQGLEWIGGIIPIFDTGNSAQSFQGRVTITADES
TSTAYMEVSSLRSDDTAVYYCASSSRIYDFTSGLAPYYDMDVWGQGTMVTVSS
SEQ ID NO: 33-1126G5 VL amino acid sequence
QAVLTQPSSVSTPPGQKVTISCSGSSSNIGNNYVSWYQQLPGTAPKLLIYDNNKRPPGIPDRFSGSKSGTSATLG
ITGLQTGDEADYYCGTWDSSLSTWVFGGGTKLTVL
SEQ ID NO: 34-1126H5 VH amino acid sequence
EVQLVQSGAEVKKPGSSVKVSCKASGGTFSTYGISWVRQAPGQGLEWIGGIIPIFDAGNSAQSFQGRVTITADES
TSTAHMEVSSLRSEDTAVYYCASSSRIYDHHIQKGGYYDMDVWGQGTMVTVSS
SEQ ID NO: 35-1126H5 VL amino acid sequence
QAVLTQPSSVSTPPGQKVTISCSGSSSNIGNNYVSWYQQLPGTAPKLLIYDNNKRPSGIPDRFSGSKSGTSATLG
ITGLQTGDEADYYCGTWDSSLSAWVFGGGTKLTVL
SEQ ID NO: 36-1127D9 VH amino acid sequence
EVQLVQSGAEVKKPGSSVKVSCKASGGTFSTYGISWVRQAPGQGLEWIGGIIPIFDTGNSAQSFQGRVTITADES
TSTAYMEVSSLRSDDTAVYYCASSSRIYDYHTIAYYD
SEQ ID NO: 37-1127D9 VL amino acid sequence
QAVLTQPSSVSTPPGQKVTISCSGSSSNIGNNYVSWYQQLPGTAPKLLIYDNNKRPSGIPDRFSGSKSGTSATLG
ITGLQTGDEADYYCGTWDSSLSAWVFGGGTKLTVL
SEQ ID NO: 38-1127F9 VH amino acid sequence
EVQLVQSGAEVKKPGSSVKVSCKASGGTFSTYGISWVRQAPGQGLEWIGGIIPIFDTGNSAQSFQGRVTITADES
TSTAYMKVSSLRSDDTAVYYCASSSRIYDYIPGMRPYYDMDVWGQGTMVTVSS
SEQ ID NO: 39-1127F9 VL amino acid sequence
QAVLTQPSSVSTPPGQKVTISCSGNSSNIGNNYVSWYQQLPGTAPKLLIYDNNKRPSGIPDRFSGSRSGTLATLG
ITGLQTGDEADYYCGTWDSSLSAWVFGGGTKLTVL
SEQ ID NO: 40-1131D7 VH amino acid sequence
EVQLVQSGAEVKKPGSSVKVSCKASGGTFSTYGISWVRQAPGQGLEWIGGIIPIFDTGNSAQSFQGRVTITADES
TSTAYMEVSSLRSDDTAVYYCASSSRIYDFNSSLIAYYDMDVWGQGTMVTVSS
SEQ ID NO: 41-1131D7 VL amino acid sequence
QAVLTQPSSVSTPPGQKVTISCSGSSSNIGNNYVSWYQQLPGTAPKLLIYDNNKRPSGIPDRFSGSKSGTSATLG
ITGLQTGDETDYYCGTWDSSLSAWVFSGGTKLTVL
SEQ ID NO: 42-1131H2 VH amino acid sequence
EVQLVQSGAEVKKPGSTVKVSCKASGGTESTYGISWVRQAPGQGLEWIGGIIPIFDTGNSAQSFQGRVTITADES
TSTAYMEVSSLRSDDTAVYYCASSSRIYDLNPSLTAYYDMDVWGQGTMVTVSS
SEQ ID NO: 43-1131H2 VL amino acid sequence
QAVLTQPSSVSTPPGQKVTISCSGTSSNIGNNYVSWYQQLPGTAPKLLIYDNNKRPSGIPDRFSGSKSGTSATLG
ITGLQTGDEADYYCGTWDSSLSAWVFGGGTKLTVL
SEQ ID NO: 44-132A9 VH amino acid sequence
EVQLVQSGAEVKKPGSSVKVSCKASGGTFSTYGISWVRQAPGQGLEWIGGIIPIFGTGNSAQSFQGRVTITADES
TSTAYMEVSSLRSDDTAVYYCASSSRIYDFEPSLIYYYDMDVWGQGTMVTVSS
SEQ ID NO: 45-132A9 VL amino acid sequence
QAVLTQPSSVSTPPGQKVTISCSGSSSNIGNNYVSWYQQLPGTAPKLLIYDNNKRPSGIPDRFSGSKSGTSATLG
ITGLQTGDEADYYCGTWDSSLSAWVFGGGTKLTVL
SEQ ID NO: 46-1132H9 VH amino acid sequence
EVQLVQSGAEVKKPGSSVKVSCKASGGTESTYGISWVRQAPGQGLEWIGGIIPIFDTGNSAQSFQGRVTITADES
TSTAYMEVSSLRSDDTAVYYCASSSRIYDLNPSLTAYYDMDVWGQGTMVTVSS
SEQ ID NO: 47-1132H9 VL amino acid sequence
QAVLTQPSSVSTPPGQKVTISCSGSSSDIGNNYVSWYQQLPGTAPKLLIYDNNKRPTGIPDRFSGSKSGTSATLG
ITGLQTGDEADYYCGTWDSSLSAWVFGGGTKLTVL
SEQ ID NO: 48-1133C11 VH amino acid sequence
EVQLVQSGAEVKKPGSSVKVSCKASGGTFSTYGISWVRQAPGQGLEWIGGIIPIFDTGNSAQSFQGRVTITADES
TSTAYMEVSSLRSDDTAVYYCASSSRIYDLNPSLTAYYDMDVWGQGTMVTVSS
SEQ ID NO: 49-1133C11 VL amino acid sequence
QAVLTQPSSVSTPPGQKVTISCSGSSSNIGNNYVSWYQQLPGTAPKLLIYDNNKRPSGIPDRFSGSKSGTSATLG
ITGLQTGDEADYYCGTWDSSLSAWVFGGGTKLTVL
SEQ ID NO: 50-1134D9 VH amino acid sequence
EVQLVQSGAEVKKPGSSVKVSCKASGGTFSTYGISWVRQAPGQGLEWIGGIIPIFDTGNSAQSFQGRVAITADES
TSTAYMEVSSLRSDDTAVYYCASSSRIYDLNPSLTAYYDMDVWGQGTMVTVSS
SEQ ID NO: 51-1134D9 VL amino acid sequence
QAVLTQPSSVSTPPGQKVTISCSGSSSNIGNNYVSWYQQLPGTAPKLLIYDNNKRPSGIPDRFSGSKSGTSATLG
ITGLQTGDEADYYCGTWDSGLSAWVFGGGTKLTVL
SEQ ID NO: 52-1145D1 VH amino acid sequence
EVQLVQSGAEVKKPGSSVKVSCKASGGTESTYGISWVRQAPGQGLEWIGGIIPIFDTSNSAQSFQGRVTITADES
TSTAYMEVSSLRSDDTAVYYCASSSRIYDFRTLYSTYYDMDVWGQGTMVTVSS
SEQ ID NO: 53-1145D1 VL amino acid sequence
QAVLTQPSSVSTPPGQKVTISCSGSSSNIGNNYVSWYQQLPGTAPKLLIYDNNKRPSGISDRFSGSKSGTSATLG
IAGLQTGDEADYYCGTWDSSLSAWVFGGGTKLTVL
SEQ ID NO: 54-1146D7 VH amino acid sequence
EVQLVQSGAEVKKPGSSVKVSCKASGGTFSTYGISWVRQAPGQGLEWIGGIIPIFDTGNSAQSFQGRVTITADES
TSTAYMEVSSLRSDDTAVYYCASSSRIYDLNPSLTAYYDMDVWGQGTMVTVSS
SEQ ID NO: 55-1146D7 VL amino acid sequence
QAVLTQPSSVSTPPGQEVTISCSGSSTNIGNNYVSWYQQLPGTAPKLLIYDNNKRPSGIPDRFSGSKSGTSATLG
ITGLQTGDEADYYCGTWDSSLSAWVFGGGTKLTVL
SEQ ID NO: 56-1147D2 VH amino acid sequence
EVQLVQSGAEVKKPGSSVRISCKASGGTFSTYGVSWVRQAPGQGLEWIGGIIPIFDTGNSAQSFQGRVTITADES
TSTAYMEVSSLRSDDTAVYYCASSSRIYDLNPSLTAYYDMDVWGQGTMVTVSS
SEQ ID NO: 57-1147D2 VL amino acid sequence
QAVLTQPSSVSTPPGQKVTISCSGSSSNIGNNYVSWYQQLPGTAPKLLIYDNNKRPSGVPDRFSGSKSGTSATLG
ITGLQTGDEADYYCGTWDSSLSAWVFGGGTKLTVL
SEQ ID NO: 58-1147G9 VH amino acid sequence
EVQLVQSGAEVKKPGSSVKVSCKASGGTFSAYGISWVRQAPGQGLEWIGGIIPIFNTGNSAQSFQGRVTITADES
TSTAYMEVSSLRSDDTAVYYCASSSRIYDLNPSLTAYYDMDVWGQGTMVTV
SEQ ID NO: 59-1147G9 VL amino acid sequence
QAVLTQPSSVSTPPGQKVTVSCSGSSSNIGNNYVSWYQQLPGTAPKLLIYDNNKRPSGIPDRFSGSKSGTSATLG
ITGLQTGDEADYYCGTWDSSLSAWVFGGGTKLTVL
SEQ ID NO: 60-1150F1 VH amino acid sequence
EVQLVQSGAEVKKPGSSVKVSCKASGGTESTYGISWVRQAPGQGLEWIGGIIPIFDTGNSAQSFQDRVTITADES
TSTAYMEVGSLRSDDTAVYYCASSSRIYDLNPSLTAYYDMDVWGHGTMVTVSS
SEQ ID NO: 61-1150F1 VL amino acid sequence
QAVLTQPSSVSTPPGQKVTISCSGSSSNIGNNYVSWYQQLPGTAPKLLIYDNNKRPSGIPDRFSGSKSGTSATLG
ITGLQTGDEADYYCGTWDSSLSAWVFGGGTKLTVL
SEQ ID NO: 62-1152H5 VH amino acid sequence
EVQLVQSGAEVKKPGSSVKVSCKASGGTFSTYGISWVRQAPGQGLVWIGGIIPIFDTGNSAQSFQGRVTITADES
TSTAYMEVSSLRSDDTAVYYCASSSRIYDMISSLQPYYDMDVWGQGTMVTVSS
SEQ ID NO: 63-1152H5 VL amino acid sequence
QAVLTQPSSVSTPPGQKATISCSGSSSNIGNNYVSWYQQLPGTAPKLLIYDNNKRPSGIPDRFSGSKSGTSATLG
ITGLQTGDEADYYCGTWDSSLSAWVFGGGTKLTVL
SEQ ID NO: 64-1155H1 VH amino acid sequence
EVQLVQSGAEVKKPGSSVKVSCKASGGTFSTYGISWVRQAPGQGLEWIGGIIPIFDTGNSAQSFQGRVTITADES
TSTAYMEVSSLRSDDTAVYYCASSSRIYDFHLANKGYYDMDVWGQGTMVTVSS
SEQ ID NO: 65-1155H1 VL amino acid sequence
QAVLTQPSSVSTPPGQKATISCSGSSSNIGNNYVSWYQQLPGTAPKLLIYDNNKRPSGIPDRFSGSKSGTSATLD
ITGLQTGDEADYYCGTWDSSLSAWVFGGGTKLTVL
SEQ ID NO: 66-1158A1 VH amino acid sequence
EVQLVQSGAEVKKPGSSVKVSCKASGGTESTYGISWVRQAPGQGLEWIGGIIPIFGTGNSAQSFQGRVTITADES
TSTAYMEVSSLRSDDTAVYYCASSSRIYDHHNHVGGYYDMDVWGQGTMVTVSS
SEQ ID NO: 67-1158A1 VL amino acid sequence
QAVLTQPSSVSTPPGQKVTISCSGSSSNIGNNYASWYQQLPGTAPKLLIYDNNKRPSGIPDRFSGSKSGTSATLG
ITGLQTGDEADYYCGTWDGSLSAWVFGGGTKLTVL
SEQ ID NO: 68-1160E3 VH amino acid sequence
EVQLVQSGAEVKKPGSSAKVSCKASGGTESTYGISWVRQAPGQGLEWIGGIIPIFDTGNSAQSFQGRVTITADES
TSTAYMEVSSLRSDDTAVYYCASSSRIYDLNPSLTAYYDMDVWGQGTMVTVSS
SEQ ID NO: 69-1160E3 VL amino acid sequence
QAVLTQPSSVSTPPGQKVTISCSGSNSNIGNNYVSWYQQLPGTAPKLLIYDNNKRPSGIPDRFSGSKSGTSATLG
ITGLQTGDEADYYCGTWDSSLSAWVFGGGTKLTV
SEQ ID NO: 70-1165D4 VH amino acid sequence
EVQLVQSGAEVKKPGSSVKVSCKASGGTESTYGISWVRQAPGQGLEWIGGIIPIFDTGNSAQSFQGRVTITADES
TSTAYMEVSSLRSDDTAVYYCASSSRIYDLNPSLTAYYDMDVWGQGTMVTVSS
SEQ ID NO: 71-1165D4 VL amino acid sequence
QAVLTQPSSVSTPPGQKVTISCSGSSSNIENNYVSWYQQLPGTAPKLLIYDNNKRPSGIPDRFSGSKSGTSATLG
ITGLQTGDEADYYCGTWDSSLSAWVFGGGTKLTVL
SEQ ID NO: 72-1175H8 VH amino acid sequence
EVQLVQSGAEVKKPGSSVKVSCKASGGTFSTYGISWVRQAPGQRLEWIGGIIPIFDTGNSAQSFQGRVTITADES
TSTAYMEVSSLRSDDTAVYYCASSSRIYDATTGLTPYYDMDVWGQGTMVTVSS
SEQ ID NO: 73-1175H8 VL amino acid sequence
QAVLTQPSSVSTPPGQKVTISCSGSSSNIGNNYVSWYQQLPGTAPKLLIYDNNKRPSGIPDRFSGSKSGTSATLG
ITGLRTGDEADYYCGTWDSSLSAWVFGGGTKLTVL
SEQ ID NO: 74-1211G10 VH amino acid sequence
EVQLVQSGAEVRKPGSSVKVSCKAYGGTFSTYGISWVRQAPGQGLEWVGGIIPIFDTRNSAQSFQGRVTITADES
TSTAYMEVSSLRSDDTAVYYCASSSRIYDMVSTLIPYYDMDVWGQGTMVTVSS
SEQ ID NO: 75-1211G10 VL amino acid sequence
QAVLTQPSSVSTPPGQKVTISCSGSSSNIGNNYVSWYQQLPGTAPKLLIYDNNKRPSGIPDRFSGSKSGTSATLG
ITGLQTGDEADYYCGTWDSSLSAWVFGGGTKLTVL
SEQ ID NO: 76-1214A1 VH amino acid sequence
EVQLVQSGAEVKKPGSSVRVSCKASGGTESTYGISWVRQAPGQGLEWIGGIIPIFDTGNSAQSFQGRVTITADES
TSTAYMEVSSLRSDDTAVYYCASSSRIYDAHLQAYYDMDVWGQGTMVTVSS
SEQ ID NO: 77-1214A1 VL amino acid sequence
QAVLTQPSSVSTPPGQKVTISCSGSSSNIGNNYVSWYQQLPGTAPKLLIYDNNKRPPGIPDRFSGSKSGTSATLG
ITGLQTGDEADYYCGTRDSSLSAWVFGGGTKLTVL
SEQ ID NO: 78-1214D10 VH amino acid sequence
EVQLVQSGAEAKKPGSSVKVSCKASGGTFSTYGISWVRQAPGRGLEWIGGIIPIFDTGNSAQSFQGRVAITADES
TSTAYMEVSSLRSDDTAVYYCASSSRIYDAHLNHHGYYDMDVWGQGTMVTVSS
SEQ ID NO: 79-1214D10 VL amino acid sequence
QAVLTQPSSVSTPPGQKVTISCSGSSSNIGNNYVSWYQQLPGTAPKLLIYDNNKRPSGIPDRFSGSKSGTSATLG
ITGLQAGDEADYYCGTWDSSLSAWVFGGGTKLTVL
SEQ ID NO: 80-1218H5 VH amino acid sequence
EVQLVQSGAVVKKPGSSVKVSCKASGGTESTYGISWVRQAPGQGLEWIGGIIPIFDTGSSAQSFQGRVTITADES
TSTAYMEVSSLRSDDTAVYYCASSSRIYDLNPSLTAYYDMDVWGQGTMVTVSS
SEQ ID NO: 81-1218H5 VL amino acid sequence
QAVLTQPSSVSTPPGQKVTISCSGSSSNTGNNYVSWYQQLSGTAPKLLIYDNNKRPSGIPDRFSGSKSGTSATLG
ITGLQTGDEADYYCGTWDSSLSAWVFGGGTKLTVL
SEQ ID NO: 82-1230H7 VH amino acid sequence
EMQLVQSGAEVKKPGSSVKVSCKASGGTFSTYGISWVRQAPGQGLEWIGGIIPIFDTGNSAQSFQGRVTITADES
TSTAYMEVSSLRSDDTAVYYCASSSRIYDENSALISYYDMDVWGQGTMVTVSS
SEQ ID NO: 83-1230H7 VL amino acid sequence
QAVLTQPSSVSTPPGQKVTISCSGSSSNIGNNYVSWYQQLPGTAPKLLIYDNNKRPSGIPDRFSGSKSGTSATLG
ITGLQTGDEADYYCGTWDSSLSAWVFGGGTKLTV
SEQ ID NO: 84-1083H4 VH amino acid sequence
QMQLVQSGAEVKKTGSSVKVSCKASGYTFAYHYLHWVRQAPGQGLEWMGGIIPIFGTTNYAQRFQDRVTITADES
TSTAYMELSSLRSEDTAVYYCASADYVWGSYRPDWYFDLWGRGTMVTVSS
SEQ ID NO: 85-1083H4 VL amino acid sequence
QSVLTQPPSASGTPGQRVTISCSGSSSNIGSNTVNWYQRLPGAAPQLLIYNNDQRPSGIPDRESGSKSGTSGSLV
ISGLQSEDEADYYCASWDDSLNGRVFGGGTKLTVL
SEQ ID NO: 86-1227H8 VH amino acid sequence
QMQLVQSGAEVKKTGSSVKVSCKASGHTFAYHYLHWVRQAPGQGLEWMGGIIPIFGTTNYAQRFQDRVTITADES
TSTAYMELSSLRSEDTAVYYCASADYAWESYQPPQINGVWGRGTMVTVSS
SEQ ID NO: 87-1227H8 VL amino acid sequence
QSVLTQPPSVSAAPGQKVTITCSGSTSNIGNNYVSWYQQHPGKAPKLMIYDVSKRPSGVPDRFSGSKSGNSASLD
ISGLQSEDEADYYCAAWDDSLSEFFFGTGTKLTVL
SEQ ID NO: 88-NGF-NG HCDR1
FGAFT
SEQ ID NO: 89-NGF-NG HCDR2
GIIPIFGLINLAQNFQG
SEQ ID NO: 90-NGF-NG HCDR3
SSRIYDLNPSLTAYYDMDV
SEQ ID NO: 91-NGF-NG LCDR1
SGSSSDIGNNYVS
SEQ ID NO: 92-NGF-NG LCDR2
DNNKRPS
SEQ ID NO: 93-NGF-NG LCDR3
GTWDSSLSAWV
SEQ ID NO: 94-MEDI-578 VH amino acid sequence with G->C
QVQLVQSGAE VKKPGSSVKV SCKASGGTFS TYGISWVRQA PGQCLEWMGG IIPIFDTGNS
AQSFQGRVTI TADESTSTAY MELSSLRSED TAVYYCARSS RIYDLNPSLT AYYDMDVWGQ
GTMVTVSS
SEQ ID NO: 95-MEDI-578 VL amino acid sequence with G->C
QSVLTQPPSV SAAPGQKVTI SCSGSSSNIG NNYVSWYQQL PGTAPKLLIY DNNKRPSGIP
DRFSGSKSGT SATLGITGLQ TGDEADYYCG TWDSSLSAWV FGCGTKLTVL
SEQ ID NO: 96-1230D8 VH amino acid sequence
QMQLVQSGAEVKKTGSSVKVSCKASGYTFPYHYLHWVRQAPGQGLEWMGGIIPIFGTTNYAQRFQDRVTITADES
TSTAYMEFSSLRSEDTAVYYCASADYVWESYHPATSLSLWGRGTMVTVSS
SEQ ID NO: 97-1230D8 VL amino acid sequence
QSVLTQPPSVSAAPGQKVTISCPGSTSNIGNNYVSWYQQRPGKAPKLMIYDVSKRPSGVPDRFSGSKSGNSASLD
ISELQSEDEADYYCAAWDDSLSEFLFGTGTKLTVL
SEQ ID NO: 98
GGGGSGGGGS
SEQ ID NO: 99-TNFR2-Fc_varB-codon optimized nucleotide sequence

| | |
|---|---|
| 1 | CTGCCCGCCC AGGTGGCCTT TACCCCTTAT GCTCCTGAGC CCGGCTCTAC CTGCCGGCTG |
| 61 | AGAGAGTACT ACGACCAGAC CGCCCAGATG TGCTGCTCCA AGTGCTCTCC TGGCCAGCAC |
| 121 | GCCAAGGTGT TCTGCACCAA GACCTCCGAT ACCGTGTGCG ACTCCTGCGA GGACTCCACC |
| 181 | TACACCCAGC TGTGGAACTG GGTGCCCGAG TGCCTGTCCT GCGGCTCCAG ATGTTCCTCC |
| 241 | GACCAGGTGG AAACCCAGGC CTGCACCAGA GAGCAGAACC GGATCTGCAC CTGTCGGCCT |
| 301 | GGCTGGTACT GCGCCCTGTC TAAGCAGGAA GGCTGCAGAC TGTGCGCCCC TCTGCGGAAG |
| 361 | TGTAGACCTG GCTTTGGCGT GGCCAGACCC GGCACCGAGA CATCTGATGT CGTGTGCAAG |
| 421 | CCTTGCGCCC CTGGCACCTT CTCCAACACC ACCTCCTCCA CCGACATCTG CCGGCCTCAC |
| 481 | CAGATCTGCA ACGTGGTGGC CATCCCTGGC AACGCCTCTA TGGACGCCGT GTGCACCTCT |
| 541 | ACCTCCCCCA CCAGAAGTAT GGCCCCTGGC GCTGTGCATC TGCCCCAGCC TGTGTCTACC |
| 601 | AGATCCCAGC ACACCCAGCC CACCCCTGAG CCTTCTACCG CCCCTTCTAC CAGCTTCCTG |
| 661 | CTGCCTATGG GCCCTAGCCC TCCTGCTGAG GGATCTACAG GCGACGAGCC CAAGTCCTGC |
| 721 | GACAAGACCC ACACCTGTCC CCCTTGTCCT GCCCCTGAAC TGCTGGGCGG ACCTTCCGTG |
| 781 | TTCCTGTTCC CCCCAAAGCC CAAGGACACC CTGATGATCA GCCGGACCCC TGAAGTGACC |
| 841 | TGCGTGGTGG TGGATGTGTC CCACGAGGAT CCCGAAGTGA AGTTCAATTG GTACGTGGAC |
| 901 | GGCGTGGAAG TGCACAACGC CAAGACCAAG CCCAGAGAGG AACAGTACAA CTCCACCTAC |
| 961 | CGGGTGGTGT CCGTGCTGAC CGTGCTGCAC CAGGATTGGC TGAACGGCAA AGAGTACAAG |
| 1021 | TGCAAGGTGT CCAACAAGGC CCTGCCTGCC CCCATCGAAA AGACCATCTC CAAGGCCAAG |
| 1081 | GGCCAGCCCC GGGAACCCCA GGTGTACACA CTGCCCCCTA GCCGGGAAGA GATGACCAAG |
| 1141 | AACCAGGTGT CCCTGACCTG TCTCGTGAAG GGCTTCTACC CCTCCGATAT CGCCGTGGAA |
| 1201 | TGGGAGTCCA ACGGCCAGCC TGAGAACAAC TACAAGACCA CCCCCCCTGT GCTGGACTCC |
| 1261 | GACGGCTCAT TCTTCCTGTA CTCCAAGCTG ACAGTGGACA AGTCCCGGTG GCAGCAGGGC |
| 1321 | AACGTGTTCT CCTGCTCCGT GATGCACGAG GCCCTGCACA ACCACTACAC CCAGAAGTCC |
| 1381 | CTGTCCCTGA GCCCTGGAAA AGGCGGCGGA GGATCTGGCG GAGGCGGATC TCAGGTGCAG |
| 1441 | CTGGTGCAGT CTGGCGCTGA AGTGAAGAAA CCCGGCTCCT CCGTGAAGGT GTCCTGCAAG |
| 1501 | GCTTCTGGCG GCACCTTCTC TACCTACGGC ATCTCCTGGG TGCGACAGGC CCCTGGCCAG |
| 1561 | TGCCTGGAAT GGATGGGCGG CATCATCCCC ATCTTCGACA CCGGCAACTC CGCCCAGAGC |
| 1621 | TTCCAGGGCA GAGTGACCAT CACCGCCGAC GAGTCTACCT CCACCGCCTA CATGGAACTG |
| 1681 | TCCTCCCTGC GGAGCGAGGA CACCGCCGTG TACTACTGCG CCCGGTCCTC TCGGATCTAC |
| 1741 | GACCTGAACC CTTCCCTGAC CGCCTACTAC GACATGGACG TGTGGGGCCA GGGCACAATG |
| 1801 | GTCACCGTGT CATCTGGTGG TGGCGGCTCT GGTGGCGGAG GAAGTGGGGG AGGGGGTTCT |
| 1861 | GGGGGGGGAG GATCTCAGTC TGTGCTGACC CAGCCTCCTT CCGTGTCTGC TGCCCCAGGC |
| 1921 | CAGAAAGTGA CAATCTCCTG CAGCGGCTCC AGCTCCAACA TCGGCAACAA CTACGTGTCC |
| 1981 | TGGTATCAGC AGCTGCCCGG CACCGCTCCC AAACTGCTGA TCTACGATAA CAACAAGCGG |
| 2041 | CCCTCCGGCA TCCCCGACAG ATTCTCCGGC TCTAAGTCCG GCACCTCTGC CACCCTGGGC |
| 2101 | ATCACCGGAC TGCAGACAGG CGACGAGGCC GACTACTACT GTGGCACCTG GGACTCCTCC |
| 2161 | CTGTCCGCTT GGGTGTTCGG CTGCGGCACC AAACTGACTG TGCTG |

The disclosure is not to be limited in scope by the specific aspects described which are intended as single illustrations of individual aspects of the disclosure, and any compositions or methods that are functionally equivalent are within the scope of this disclosure. Indeed, various modifications of the disclosure in addition to those shown and described herein will become apparent to those skilled in the art from the foregoing description and accompanying drawings. Such modifications are intended to fall within the scope of the appended claims.

All publications and patent applications mentioned in this specification are herein incorporated by reference to the same extent as if each individual publication or patent application was specifically and individually indicated to be incorporated by reference.

1. A method of reducing or preventing pain in a subject in need thereof, the method comprising administering to the subject a subcutaneous fixed dose of a binding molecule, wherein the binding molecule comprises an NGF antagonist domain and a TNFα antagonist domain, wherein the NGF antagonist domain is an anti-NGF antibody or an antigen-binding fragment thereof, wherein the TNFα antagonist domain comprises a soluble TNFα binding fragment of TNFR, and wherein the method reduces or prevents pain in the subject.

2. The method of claim 1, wherein the subcutaneous fixed dose of the binding molecule is about 5 to 200 mg or about 7.5 to 150 mg.

3. (canceled)

4. The method of claim 1, wherein the subcutaneous fixed dose of the binding molecule is about 7.5 mg, about 25 mg, about 75 mg or about 150 mg.

5. The method of claim 1, wherein the subcutaneous fixed dose is equivalent to an intravenous fixed dose of 30 mg of the binding molecule.

6. The method of claim 1, wherein the fixed dose is administered at least once every two weeks.

7. The method of claim 1, wherein the fixed dose is administered for at least 12 weeks.

8. The method of claim 1, wherein the pain comprises chronic pain, osteoarthritic pain or osteoarthritic pain of the knee.

9. (canceled)

10. (canceled)

11. The method of claim 1, wherein the subject has suffered the pain for 3 months or longer prior to administration with the binding molecule.

12. The method of claim 1, wherein the pain is associated with joint inflammation.

13. The method of claim 1, wherein the subject has osteoarthritis.

14. The method of claim 13, wherein the subject has unilateral osteoarthritis of the knee.

15. The method of claim 13, wherein the subject has at least Grade 2 osteoarthritis of the knee joint on the Kellgren-Lawrence (KL) grading scale of 0 to 4 as per central reader evaluation.

16. The method of claim 1, comprising the following steps prior to administration of the binding molecule to the subject:
a. administering to the subject a NSAID, strong opioid, weak opioid, COX-2 inhibitor, acetaminophen or a combination thereof, and
b. determining i) that the NSAID, strong opioid, weak opioid, COX-2 inhibitor, acetaminophen or a combination thereof does not reduce or prevent pain in the subject, and/or ii) determining that the subject is intolerant to the NSAID, strong opioid, weak opioid, COX-2 inhibitor, acetaminophen or a combination thereof.

17. The method of claim 16, wherein the NSAID, strong opioid, weak opioid, COX-2 inhibitor, acetaminophen or a combination thereof is administered for at least 2 weeks.

18. The method of claim 16, wherein the NSAID, strong opioid, weak opioid, COX-2 inhibitor, acetaminophen or a combination thereof has been administered to the subject for at least 2 weeks prior to administration with the binding molecule.

19. The method of claim 1, wherein the subject is intolerant to NSAIDs, strong opioids, weak opioids, COX-2 inhibitors, acetaminophen or a combination thereof.

20. The method of claim 1, wherein the method comprises testing the subject for SARS-CoV2 infection prior to administration with the fixed dose of the binding molecule.

21. The method of claim 20, wherein testing the subject for SARS-CoV2 infection comprises testing the subject for SARS-CoV2 genetic material prior to administration with the fixed dose of the binding molecule.

22. The method of claim 1, wherein the subject is not infected with SARS-CoV2 at baseline.

23. The method of claim 1, wherein the subject has a mean Western Ontario and McMaster Universities Osteoarthritis (WOMAC) pain score of at least 5 in a joint as measured using the pain subscale of the WOMAC index at baseline.

24. The method of claim 1, wherein the subject has a mean pain intensity score of at least 5 in a joint as measured on a pain numerical rating scale (NRS) at baseline.

25. The method of claim 1, wherein the method reduces the subject's weekly average of daily NRS pain score from baseline.

26. The method of claim 1, wherein the fixed dose is administered every 2 weeks for 12 weeks, and wherein the method reduces the subject's weekly average of daily NRS pain score from baseline by at least week 12.

27. The method of claim 1, wherein the method reduces the subject's weekly average of daily NRS pain score from baseline by at least 30% or by at least 50%.

28. (canceled)

29. The method of claim 1, wherein the method reduces the subject's WOMAC pain subscale score from baseline.

30. The method of claim 1, wherein the fixed dose is administered every 2 weeks for 12 weeks, and wherein the method reduces the subject's WOMAC pain subscale score from baseline by at least week 12.

31. The method of claim 1, wherein the method reduces the subject's WOMAC pain subscale score from baseline by at least 30% or by at least 50%.

32. (canceled)

33. The method of claim 1, wherein the method reduces the subject's WOMAC physical subscale score from baseline by at least 30% or by at least 50%.

34. (canceled)

35. The method of claim 1, wherein the method improves the Patient Global Assessment (PGA) of osteoarthritis from baseline.

36. The method of claim 1, wherein the fixed dose is administered every 2 weeks for 12 weeks, and wherein method improves the PGA of osteoarthritis from baseline by at least week 12.

37. The method of claim 1, wherein the method improves the PGA of osteoarthritis by at least 2 points.

38. The method of claim 1, wherein pain reduction is observed following a single dose administration of the binding molecule in the subject.

39. The method of claim 1, wherein the method comprises administering an NSAID, an opioid, acetaminophen, and/or a COX-2 inhibitor to the subject.

40. (canceled)

41. (canceled)

42. (canceled)

43. The method of claim 1, wherein the anti-NGF antibody or fragment thereof can inhibit NGF binding to TrkA, p75NRT, or both TrkA and P75NRT.

44. The method of claim 1, wherein the anti-NGF antibody or fragment thereof preferentially blocks NGF binding to TrkA over NGF binding to p75NRT.

45. The method of claim 1, wherein the anti-NGF antibody or fragment thereof binds human NGF with an affinity of about 0.25-0.44 nM.

46. The method of claim 1, wherein the anti-NGF antibody or fragment thereof comprises an antibody VH domain comprising a set of CDRs HCDR1, HCDR2, HCDR3 and an antibody VL domain comprising a set of CDRs LCDR1, LCDR2 and LCDR3, wherein the HCDR1 has the amino acid sequence of SEQ ID NO: 4 or SEQ ID NO: 4 with up to two amino acid substitutions, the HCDR2 has the amino acid sequence of SEQ ID NO: 5 or SEQ ID NO: 5 with up to two amino acid substitutions, the HCDR3 has the amino acid sequence of SEQ ID NO: 6 or SEQ ID NO: 6 with up to two amino acid substitutions, SSRIYDFNSALISYYDMDV (SEQ ID NO: 11), or SSRIYDMISSLQPYYDMDV (SEQ ID NO:12), the LCDR1 has the amino acid sequence of SEQ ID NO: 8 or SEQ ID NO: 8 with up to two amino acid substitutions, the LCDR2 has the amino acid sequence of SEQ ID NO: 9 or SEQ ID NO: 9 with up to two amino acid substitutions, and the LCDR3 has the amino acid sequence of SEQ ID NO: 10 or SEQ ID NO: 10 with up to two amino acid substitutions.

47. The method of claim 1, wherein the anti-NGF antibody or fragment thereof comprises an antibody VH domain comprising a set of CDRs HCDR1, HCDR2, HCDR3 and an antibody VL domain comprising a set of CDRs LCDR1, LCDR2 and LCDR3, wherein the HCDR1 comprises the amino acid sequence of SEQ ID NO: 4,
the HCDR2 comprises the amino acid sequence of SEQ ID NO: 5,
the HCDR3 comprises the amino acid sequence of SEQ ID NO: 6, SSRIYDFNSALISYYDMDV (SEQ ID NO: 11), or SSRIYDMISSLQPYYDMDV (SEQ ID NO:12),
the LCDR1 comprises the amino acid sequence of SEQ ID NO: 8,
the LCDR2 comprises the amino acid sequence of SEQ ID NO: 9; and
the LCDR3 comprises the amino acid sequence of SEQ ID NO: 10.

48. The method of claim 1, wherein the anti-NGF antibody or fragment thereof comprises a VH having an amino acid sequence that is at least 80%, 85%, 90%, 95%, 97%, 99% or 100% identical to the amino acid sequence of SEQ ID NO: 3 or 94.

49. The method of claim 1, wherein the anti-NGF antibody or fragment thereof comprises a VL having an amino acid sequence that is at least 80%, 85%, 90%, 95%, 97%, 99% or 100% identical to the amino acid sequence of SEQ ID NO: 7 or 95.

50. The method of claim 1, wherein the anti-NGF antibody or fragment thereof is a full H₂L₂ antibody, a Fab, fragment, an Fab′ fragment, an F(ab)₂ fragment or a single chain Fv (scFv) fragment.

51. The method of claim 1, wherein the anti-NGF antibody or fragment thereof is humanized, chimeric, primatized, or fully human.

52. The method of claim 1, wherein the anti-NGF scFv fragment comprises, from N-terminus to C-terminus, a VH comprising an amino acid sequence that is at least 80%, 85%, 90%, 95%, 97%, 99% or 100% identical to the amino acid sequence of SEQ ID NO: 3, a 15-amino acid linker sequence (GGGGS)₃ (SEQ ID NO: 15), and a VL comprising an amino acid sequence that is at least 80%, 85%, 90%, 95%, 97%, 99% or 100% identical to the amino acid sequence of SEQ ID NO: 7.

53. The method of claim 1, wherein the anti-NGF scFv fragment comprises, from N-terminus to C-terminus, a VH comprising an amino acid sequence that is at least 80%, 85%, 90%, 95%, 97%, 99% or 100% identical to the amino acid sequence of SEQ ID NO: 94, a 20-amino acid linker sequence (GGGGS)₄ (SEQ ID NO:19), and a VL comprising an amino acid sequence that is at least 80%, 85%, 90%, 95%, 97%, 99% or 100% identical to the amino acid sequence of SEQ ID NO: 95.

54. The method of claim 1, wherein the TNFR is TNFR-2.

55. The method of claim 54, wherein the TNFR-2 fragment is fused to an immunoglobulin Fc domain.

56. The method of claim 55, wherein the immunoglobulin Fc domain is a human IgG1 Fc domain.

57. The method of claim 1, wherein the TNFα antagonist comprises an amino acid sequence that is at least 80%, 85%, 90%, 95%, 97%, 99% or 100% identical to the amino acid sequence set forth in SEQ ID NO: 13, or a functional fragment thereof.

58. The method of claim 1, wherein the binding molecule comprises a fusion protein that comprises the NGF antagonist fused to the TNFα antagonist through a linker.

59. The method of claim 1, wherein the binding molecule comprises a homodimer of the fusion protein.

60. The method of claim 1, wherein the binding molecule comprises a homodimer of a fusion polypeptide comprising, from N-terminus to C-terminus, a TNFα-binding fragment of TNFR-2 comprising an amino acid sequence that is at least 80%, 85%, 90%, 95%, 97%, 99% or 100% identical to a sequence corresponding to amino acids 1-235 of SEQ ID NO: 13, a human IgG1Fc domain, a 10 amino-acid linker sequence (GGGGS)₂(SEQ ID NO: 98), a VH comprising an amino acid sequence that is at least 80%, 85%, 90%, 95%, 97%, 99% or 100% identical to the amino acid sequence of SEQ ID NO 3 or 94, a 15-amino acid linker sequence (GGGGS)₃ (SEQ ID NO: 15), and a VL comprising an amino acid sequence that is at least 80%, 85%, 90%, 95%, 97%, 99% or 100% identical to the amino acid sequence of SEQ ID NO: 7 or 95.

61. The method of claim 1, wherein the binding molecule comprises a homodimer of a fusion polypeptide comprising an amino acid sequence that is at least 80%, 85%, 90%, 95%, 97%, 99% or 100% identical to the amino acid sequence of SEQ ID NO: 14.

62. The method of claim 1, wherein the binding molecule comprises a homodimer of a fusion polypeptide comprising, from N-terminus to C-terminus, a TNFα-binding 75 kD fragment of TNFR-2 comprising the amino acid sequence of SEQ ID NO: 13, a linker sequence (GGGGS₂ (SEQ ID NO: 98), a VH comprising the amino acid sequence of SEQ ID NO: 94, a 20-amino acid linker sequence (GGGGS)₄ (SEQ ID NO: 19), and a VL comprising the amino acid sequence of SEQ ID NO: 95.

63. The method of claim 1, wherein the glycine residue at the amino acid position corresponding to position 102, 103, or 104 of SEQ ID NO: 7 is modified to a cysteine residue, and wherein the glycine residue at the amino acid position corresponding to position 44 of SEQ ID NO: 3 is modified to a cysteine residue.

64. (canceled)

65. The method of claim 1, wherein the binding molecule comprises a homodimer of a fusion polypeptide comprising an amino acid sequence that is at least 80%, 85%, 90%, 95%, 99% or 100% identical to the amino acid sequence of SEQ ID NO: 17.

66. (canceled)

67. (canceled)
